(19) United States
(12) Patent Application Publication
(10) Pub. No.: US 2015/0291590 A1
(43) Pub. Date: Oct. 15, 2015

(54) COMPOSITIONS AND METHODS FOR THE TREATMENT OF METABOLIC DISEASES

(71) Applicant: CELLIXBIO PRIVATE LIMITED, Andhra Pradesh (IN)

(72) Inventor: Mahesh Kandula, East Godavari District, Andhra Pradesh (IN)

(21) Appl. No.: 14/399,530

(22) PCT Filed: Aug. 18, 2013
(86) PCT No.: PCT/IB2013/056715

§ 371 Date: Nov. 7, 2014

(30) Foreign Application Priority Data
Sep. 17, 2012 (IN) 3866/CHE/2012

(51) Int. Cl. C07D 475/04

(52) U.S. Cl. CPC C07D 475/04

(57) ABSTRACT

The invention relates to the compounds of formula I, formula II and formula III or its pharmaceutical acceptable salts, as well as polymorphs, solvates, enantiomers, stereoisomers and hydrates thereof. The pharmaceutical compositions comprising an effective amount of compounds of formula I, formula II or formula III; and methods for treating or preventing metabolic diseases may be formulated for oral, buccal, rectal, topical, transdermal, transmucosal, intravenous, parenteral administration, syrup, or injection. Such compositions may be used to treatment of phenylketonuria, cardiovascular disease, autism, ADHD, hypertension, endothelial dysfunction and chronic kidney disease.

### PRIORITY

The present application claims the benefit of Indian Provisional Patent Application No. 3866/CHE/2012 filed on 17 Sep. 2012 and the International Application No. PCT/M2013/056715 filed on 18 Aug. 2013, the entire disclosure of which is relied on for all purposes and is incorporated into this application by reference.

### FIELD OF THE INVENTION

This disclosure generally relates to compounds and compositions for the treatment of metabolic diseases. More particularly, this invention relates to treating subjects with a pharmaceutically acceptable dose of compounds, crystals, solvates, enantiomer, stereoisomer, esters, salts, hydrates, prodrugs, or mixtures thereof.

### BACKGROUND OF THE INVENTION

Tetrahydrobiopterin (BH4) was subsequently found to be an essential cofactor for several other aromatic amino acid hydroxylases (tyrosine and tryptophane) involved with neurotransmitter biosynthesis, glyceryl-ether mono-oxygenase, and nitric oxide synthase (NOS). To be functional, BH4 must be in its fully reduced form, and depletion and/or BH4 oxidation to BH3 and BH2 reduces its activity. For the cardiovascular system, the role of BH4 in NOS activity is particularly relevant. Reduced BH4 was first shown to contribute to vascular pathophysiology and hypertension, whereas more recent studies have found important roles in cardiac hypertrophy and remodeling, and ischemia/reperfusion physiology.

Elevated blood Phenylalanine (Phe), such as defects in the production or regeneration of the cofactor tetrahydrobiopterin (BH4). This cofactor is involved in the hydroxylation of Phe to tyrosine by PAH. However, the cofactor BH4 is also utilized in the hydroxylation reactions of tyrosine, and tryptophan. The inability to hydroxylate these amino acids leads to deficient levels of the neurotransmitters L-DOPA, from tyrosine, and serotonin, from tryptophan. Treatment of cofactor defects is completely different from for Phenylketonuria (PKU). Patients with PKU are able to make adequate amounts of BH4 and have normal regeneration of BH4.

BH4 is an essential cofactor for all 3 NOS isoforms, and basal enzyme activity correlates with the amount of BH4 bound tightly to the protein. NOS is a homodimeric oxidoreductase containing iron protoporphyrin IX (heme), flavin adenine dinucleotide, flavin mononucleotide, and BH4.

Clinical data supporting vascular benefits of exogenous BH4 are largely based on acute or subacute studies examining endothelium-dependent vasodilation by agonists or flow stimuli. BH4 improves endothelial function in those who smoke, diabetic subjects, hypertensive subjects, patients with hypercholesterolemia, and those with coronary artery disease. Unlike hypertension, hypertrophy, and oxidant stress stimulation, other stimuli such as inflammatory cytokines have been found to increase BH4 biosynthesis, and this may play a role in atherosclerosis.

Managing acute pathology of often relies on the addressing underlying pathology and symptoms of the disease. There is currently a need in the art for new compositions to treatment or delay of the onset of metabolic diseases and its associated complications progression.

### SUMMARY OF THE INVENTION

The present invention provides compounds, compositions containing these compounds and methods for using the same to treat, prevent and/or ameliorate the effects of the conditions such as metabolic diseases.

The invention herein provides compositions comprising of formula I or pharmaceutical acceptable salts thereof. The invention also provides pharmaceutical compositions comprising one or more compounds of formula I or intermediates thereof and one or more of pharmaceutically acceptable carriers, vehicles or diluents. These compositions may be used in the treatment of metabolic diseases and its associated complications.

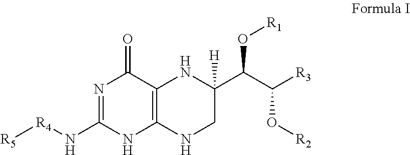

In certain embodiments, the present invention relates to the compounds and compositions of formula I, or pharmaceutically acceptable salts thereof,

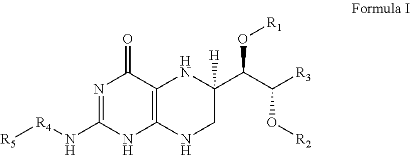

and pharmaceutically acceptable salts, hydrates, solvates, prodrugs, enantiomers, and stereoisomers thereof;

Wherein,
R¹, R², R³ each independently represents H, D, CH₃CO, CD₃CO, CD₃, CH₃,

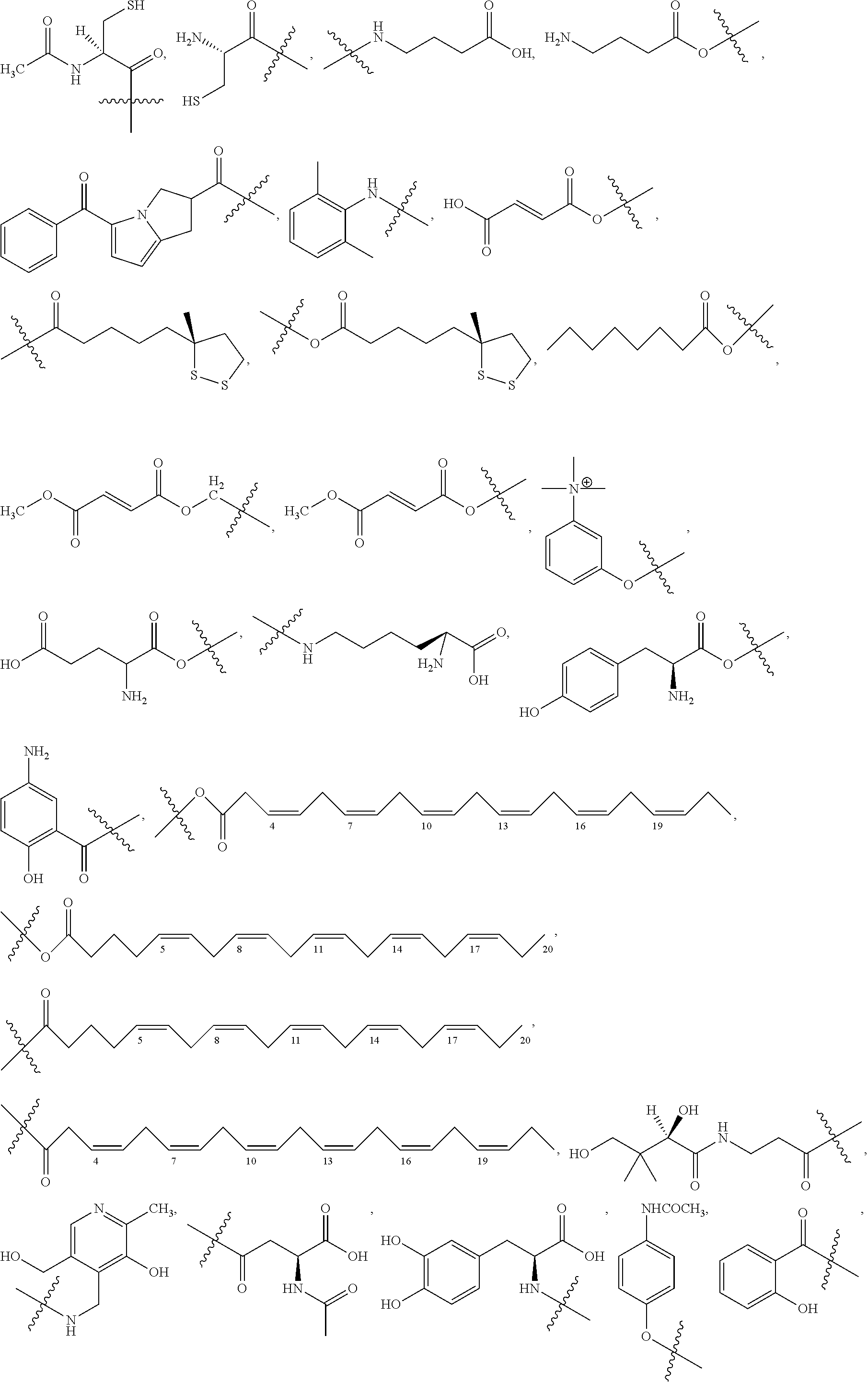

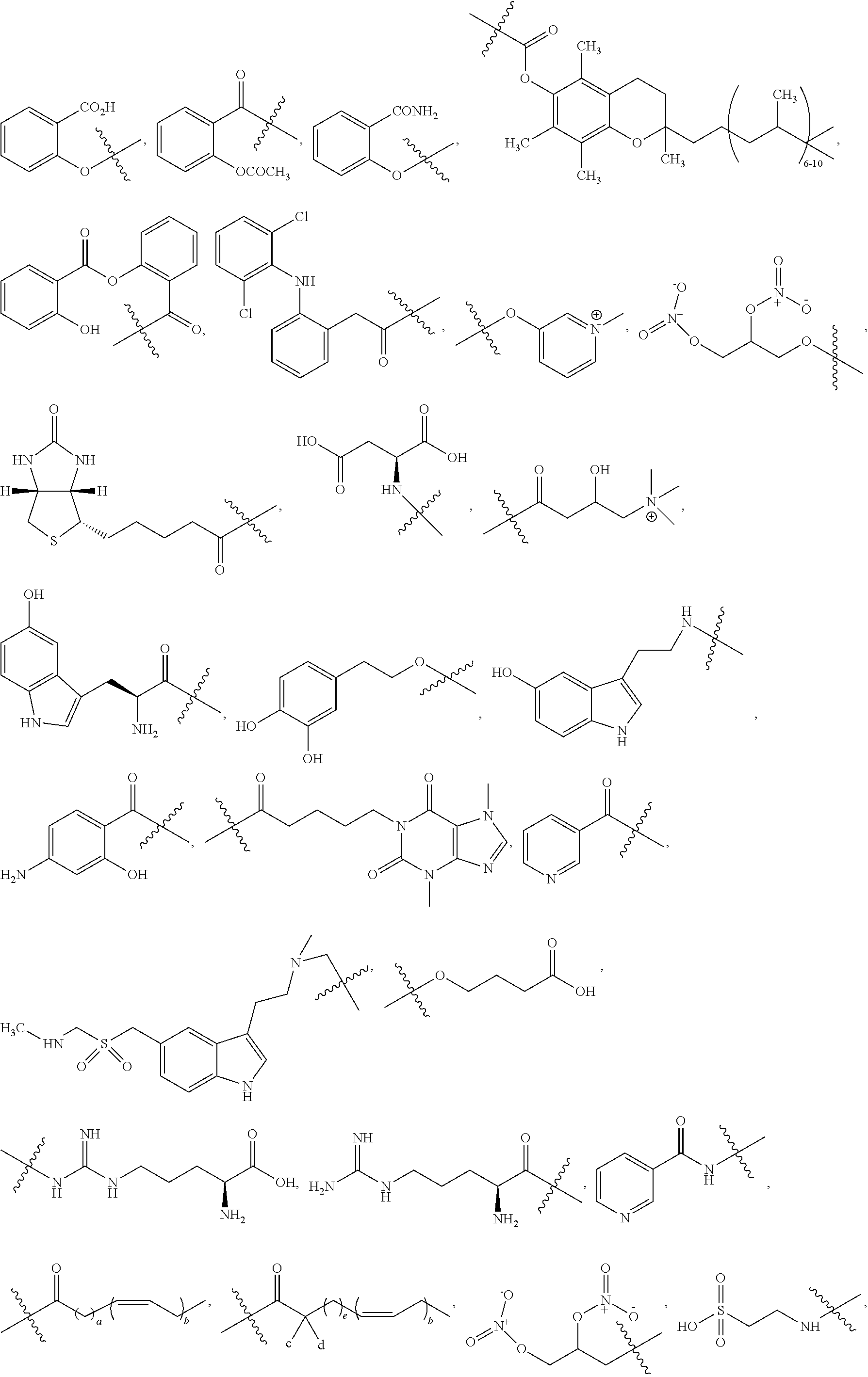

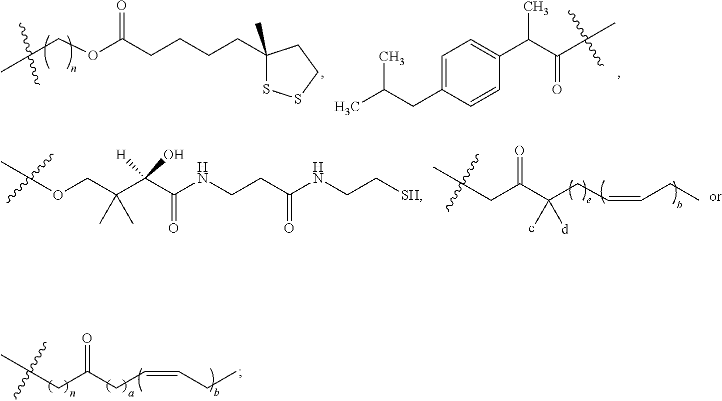

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;
   R⁴ represents H, D, CH₃CO, CD₃CO, CD₃, CH₃,

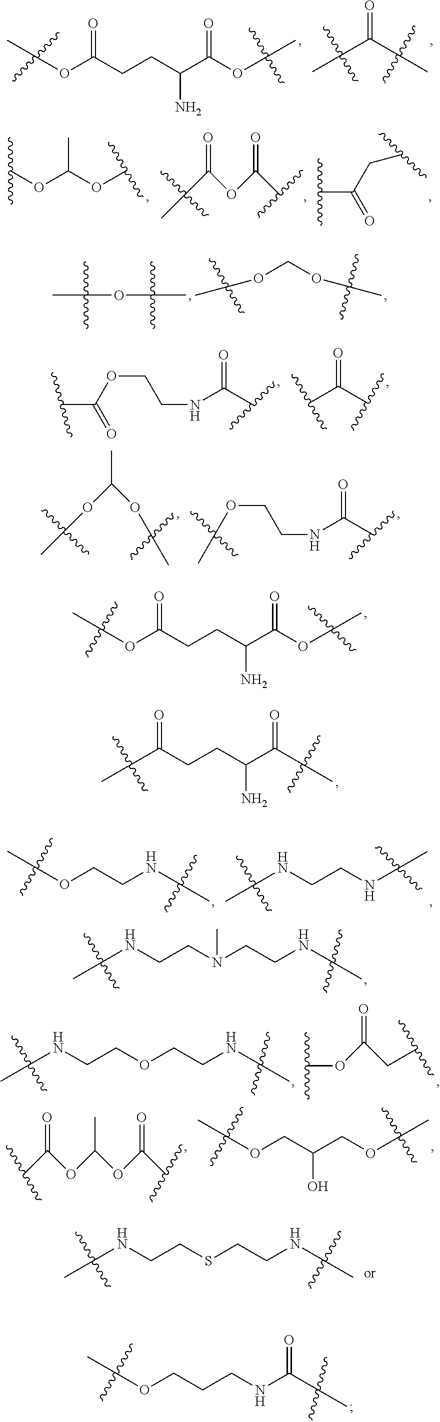

R⁵ represents D, CH₃CO, CD₃CO, CD₃,

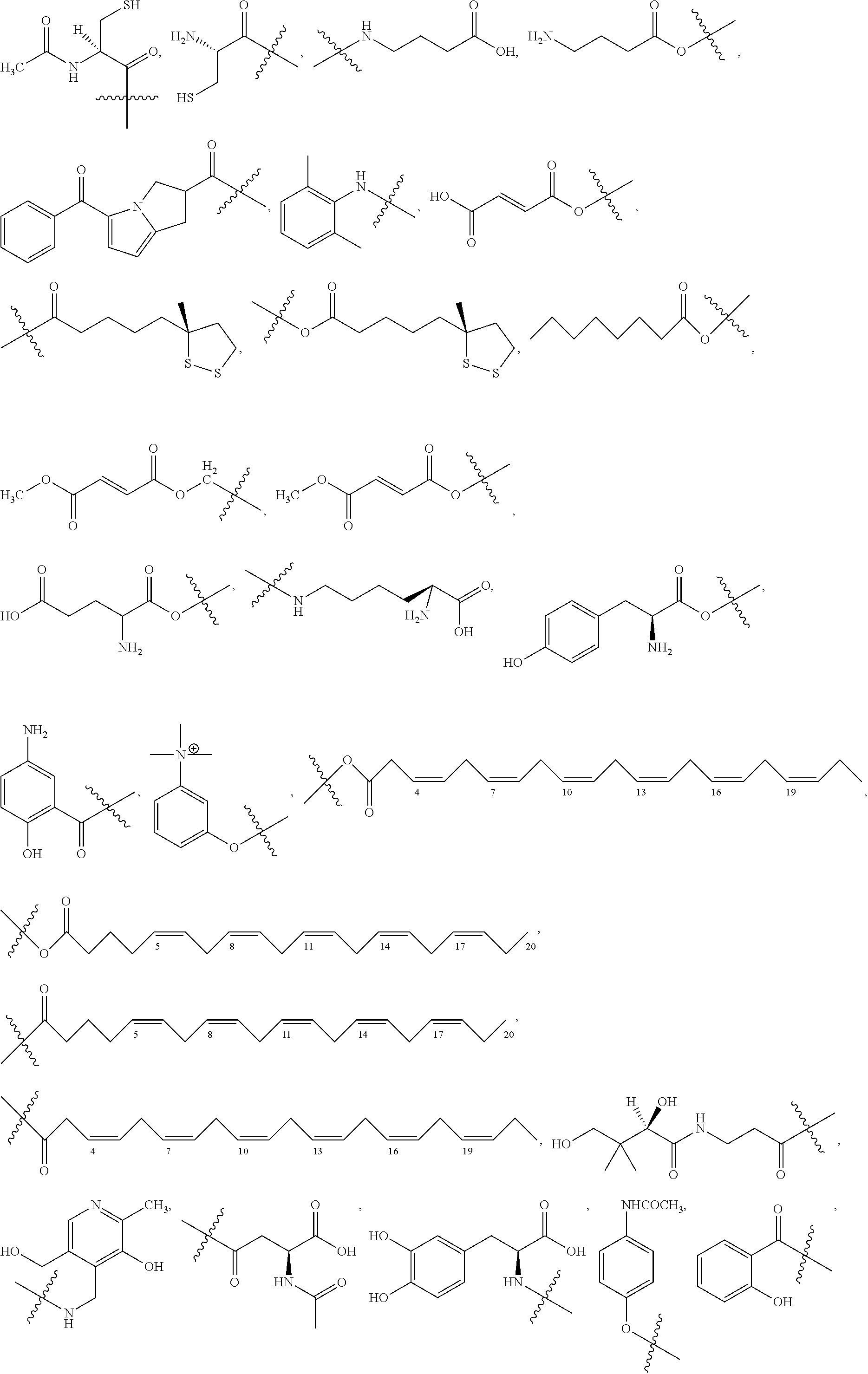

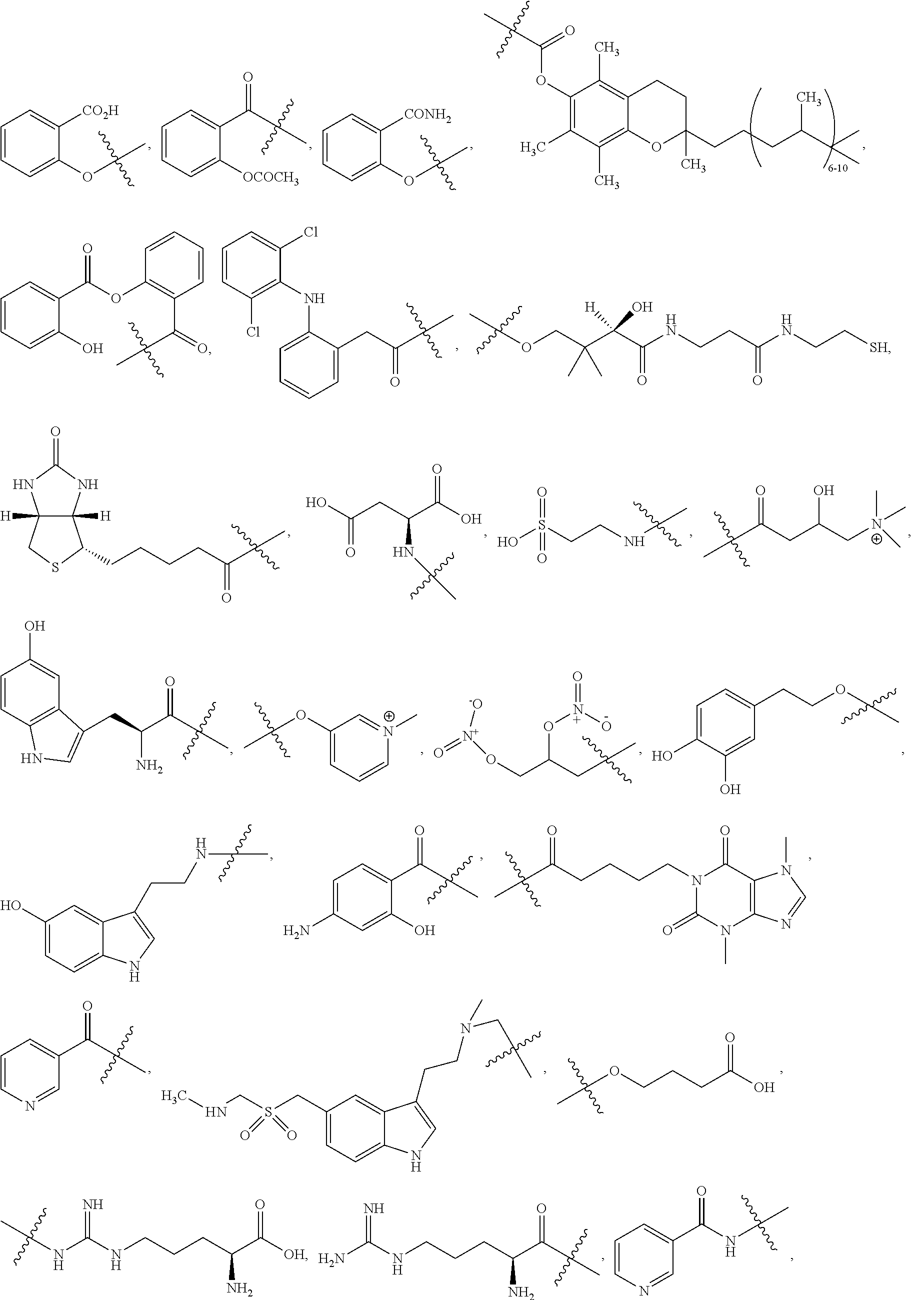

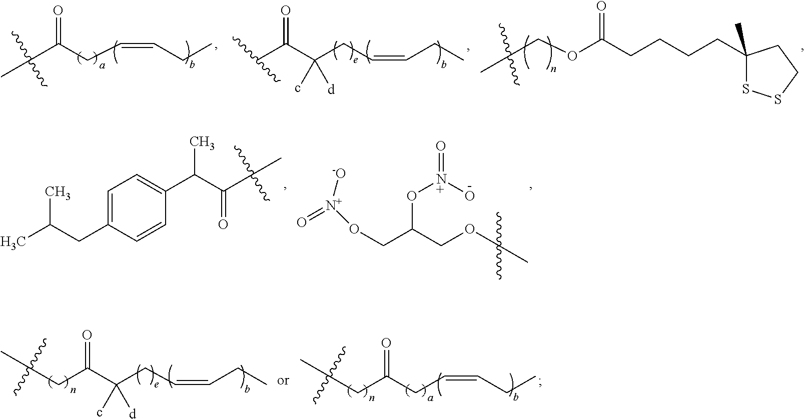

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃.

In one aspect, compounds of the Formula II are described:

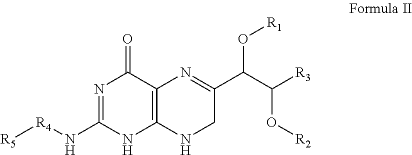

and pharmaceutically acceptable salts, hydrates, solvates, prodrugs, enantiomers, and stereoisomers thereof;

Wherein,
R¹, R², R³ each independently represents H, D, CH₃CO, CD₃CO, CD₃, CH₃,

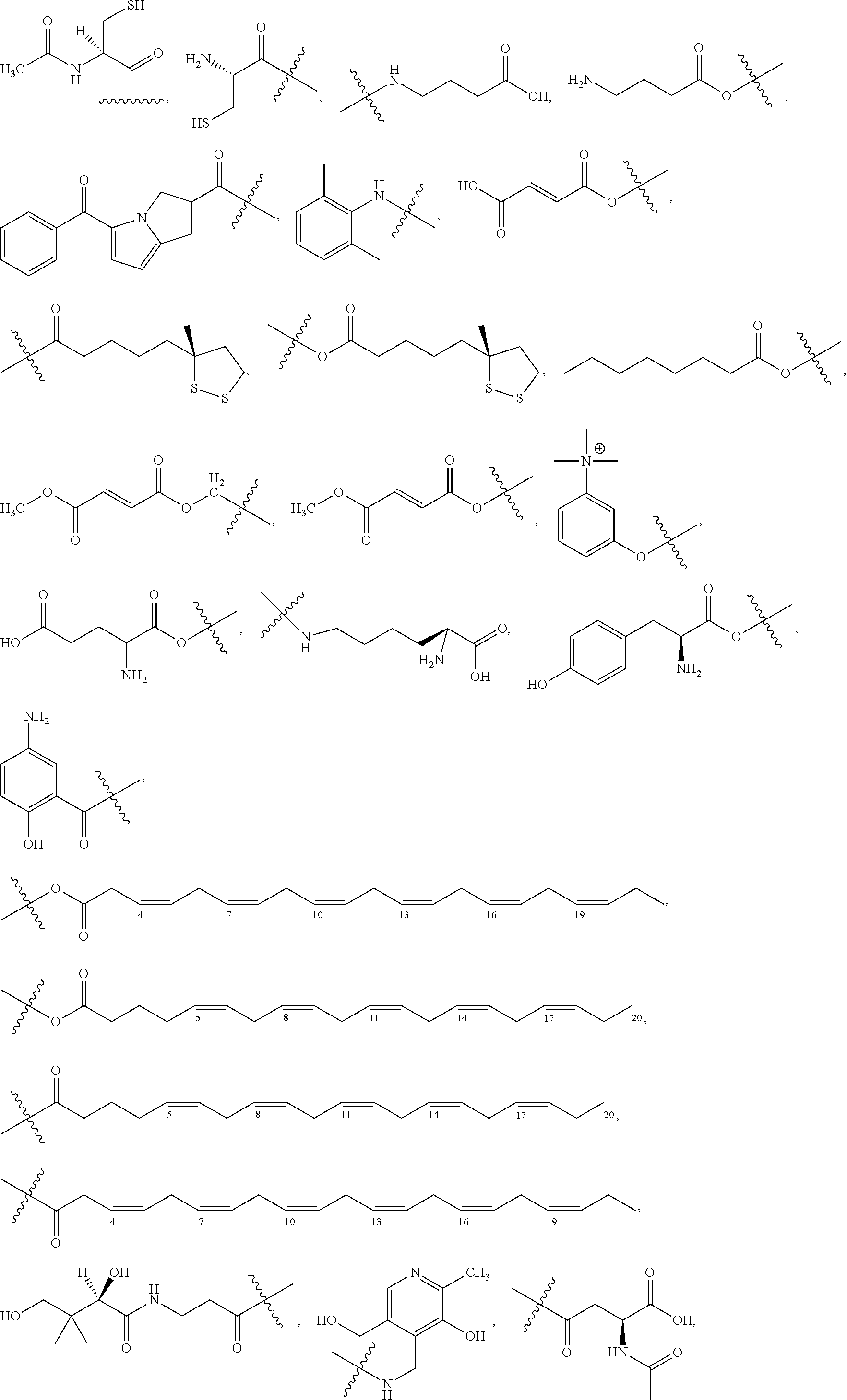

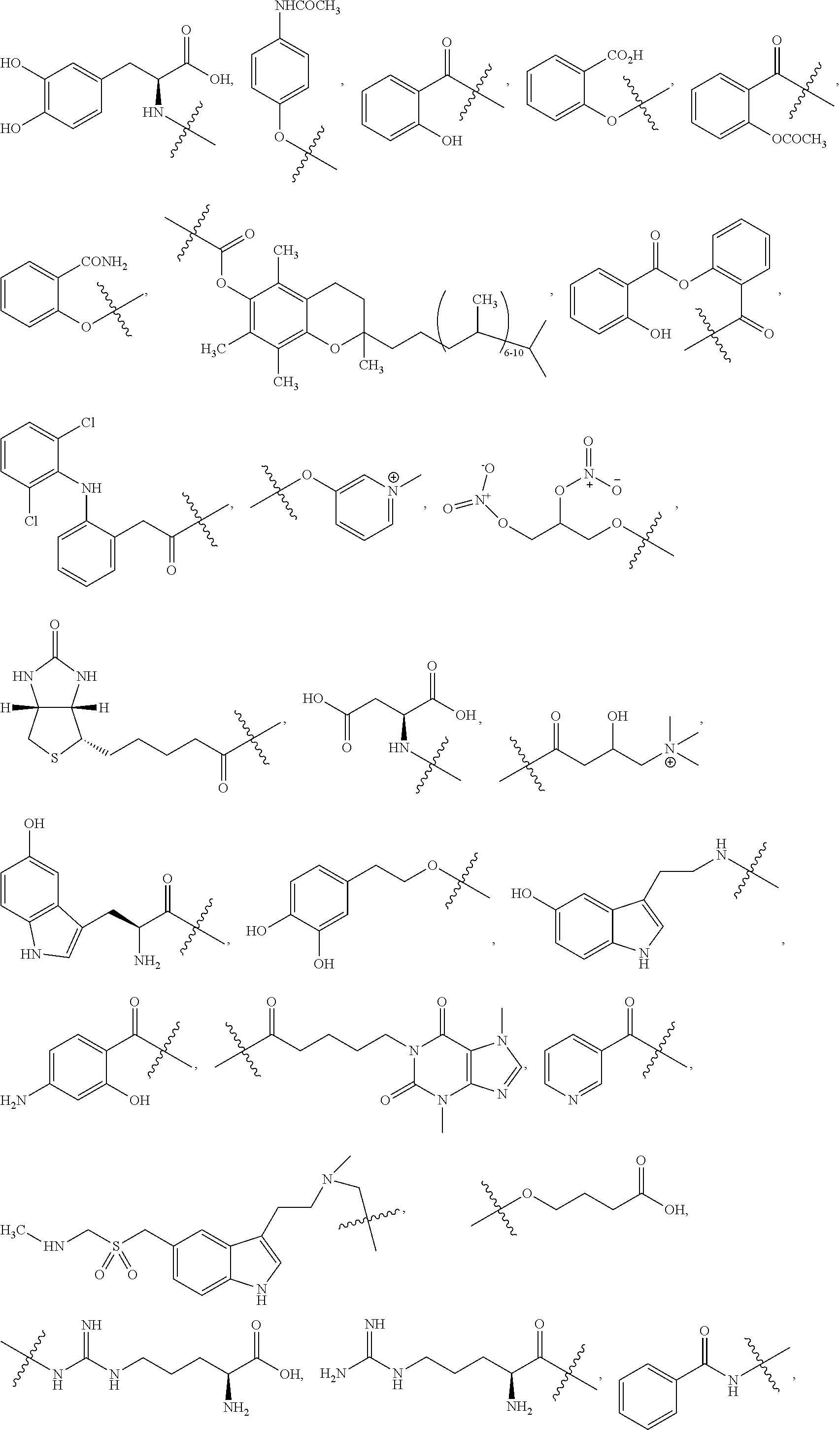

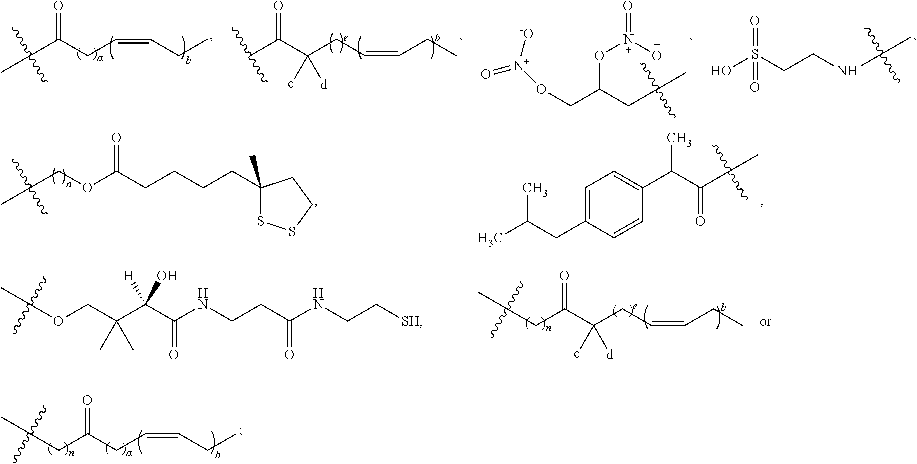

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;
   R⁴ represents H, D, CH₃CO, CD₃CO, CD₃, CH₃,

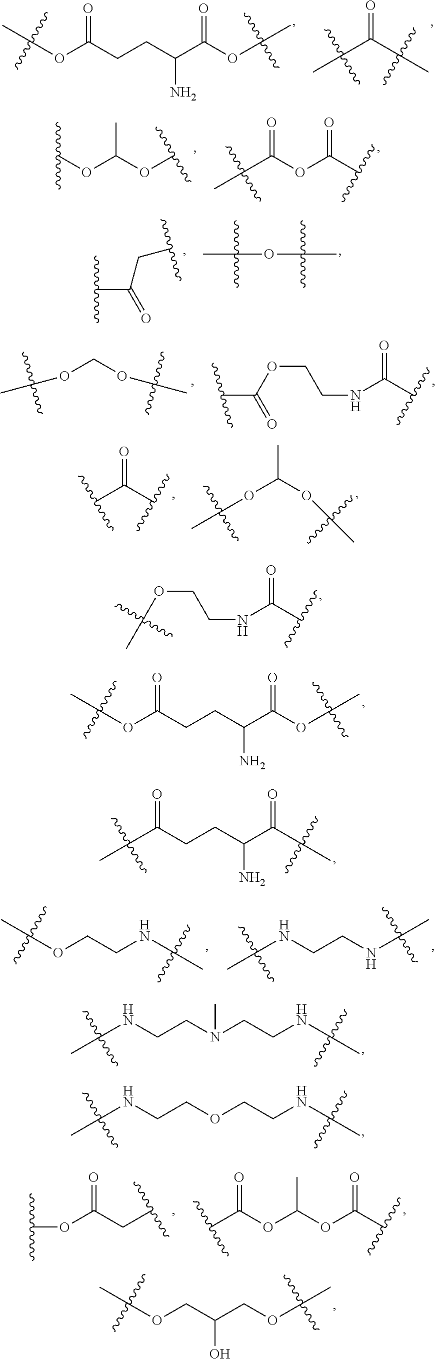

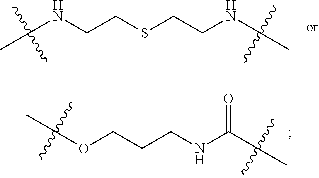 [SMILES: *NCCSCCN* ; *OCCCNC(*)=O]

R⁵ represents D, CH₃CO, CD₃CO, CD₃,

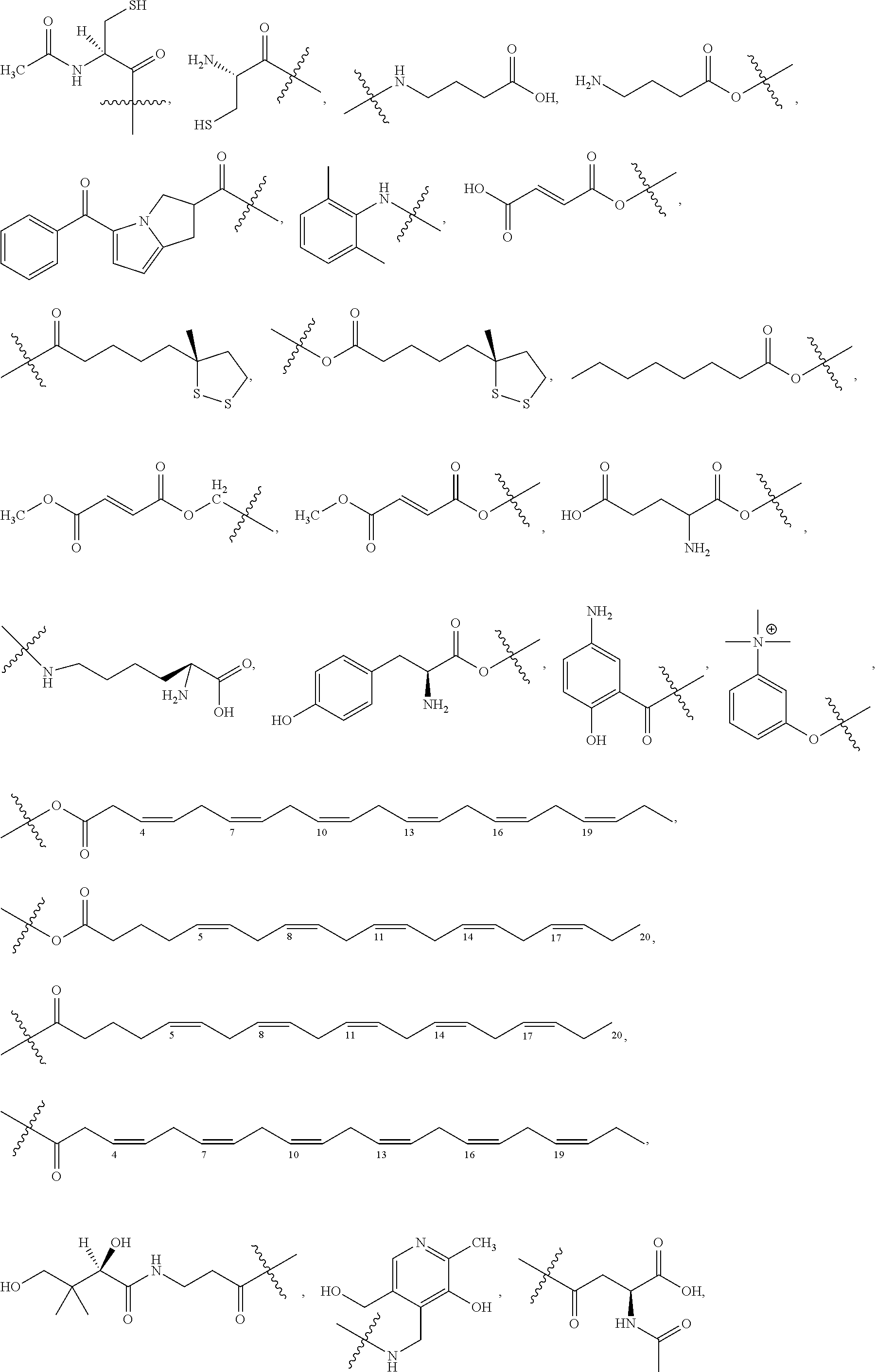

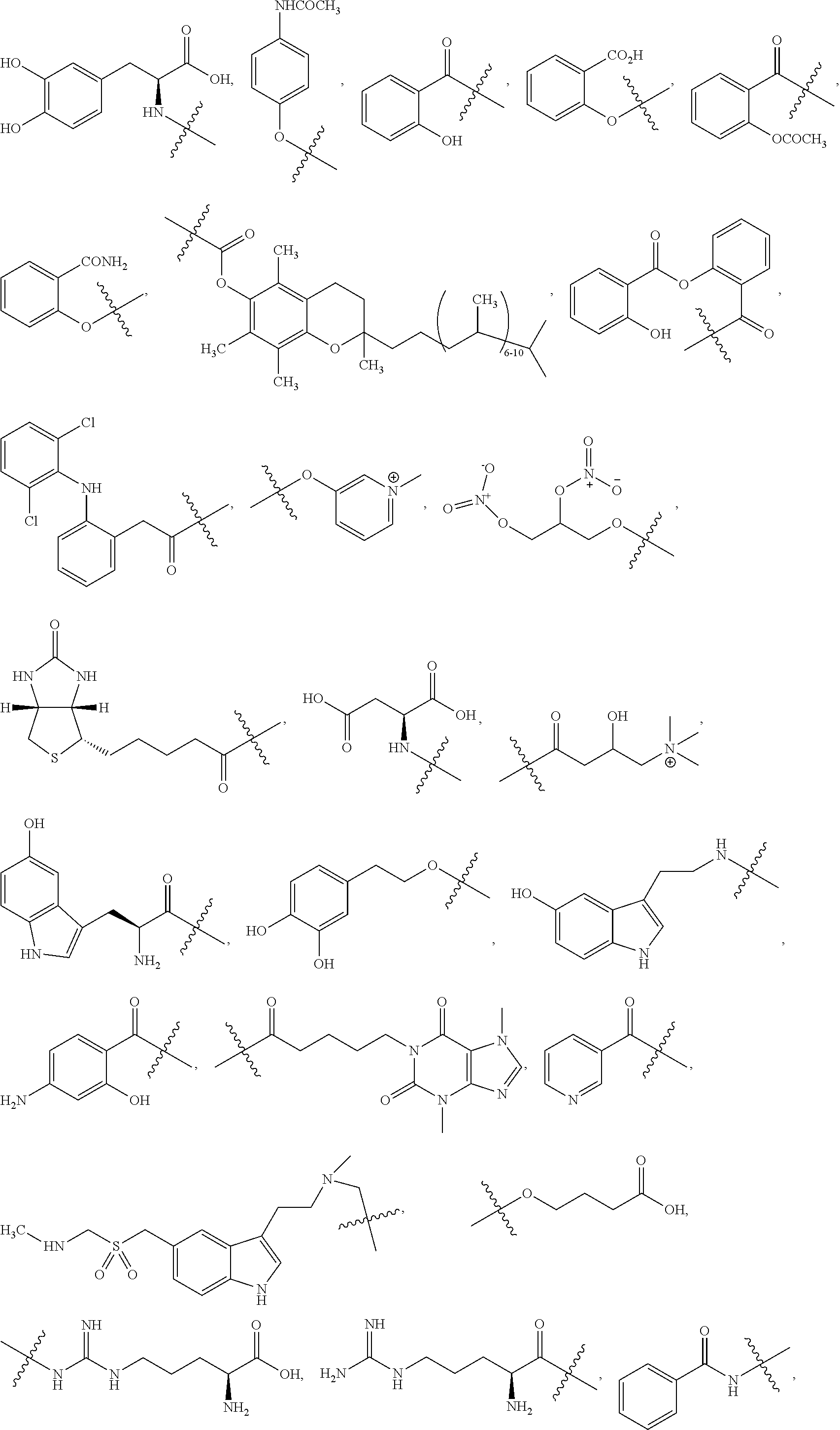

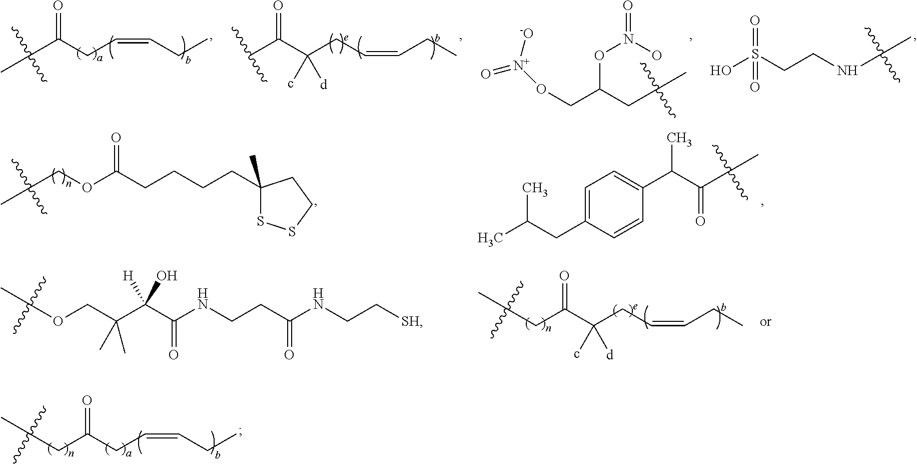

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃.

In another aspect, compounds of the Formula III are described:

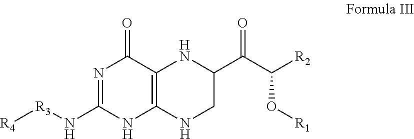

and pharmaceutically acceptable salts, hydrates, solvates, prodrugs, enantiomers, and stereoisomers thereof;

Wherein,
R¹, R² each independently represents H, D, CH₃CO, CD₃CO, CD₃, CH₃,

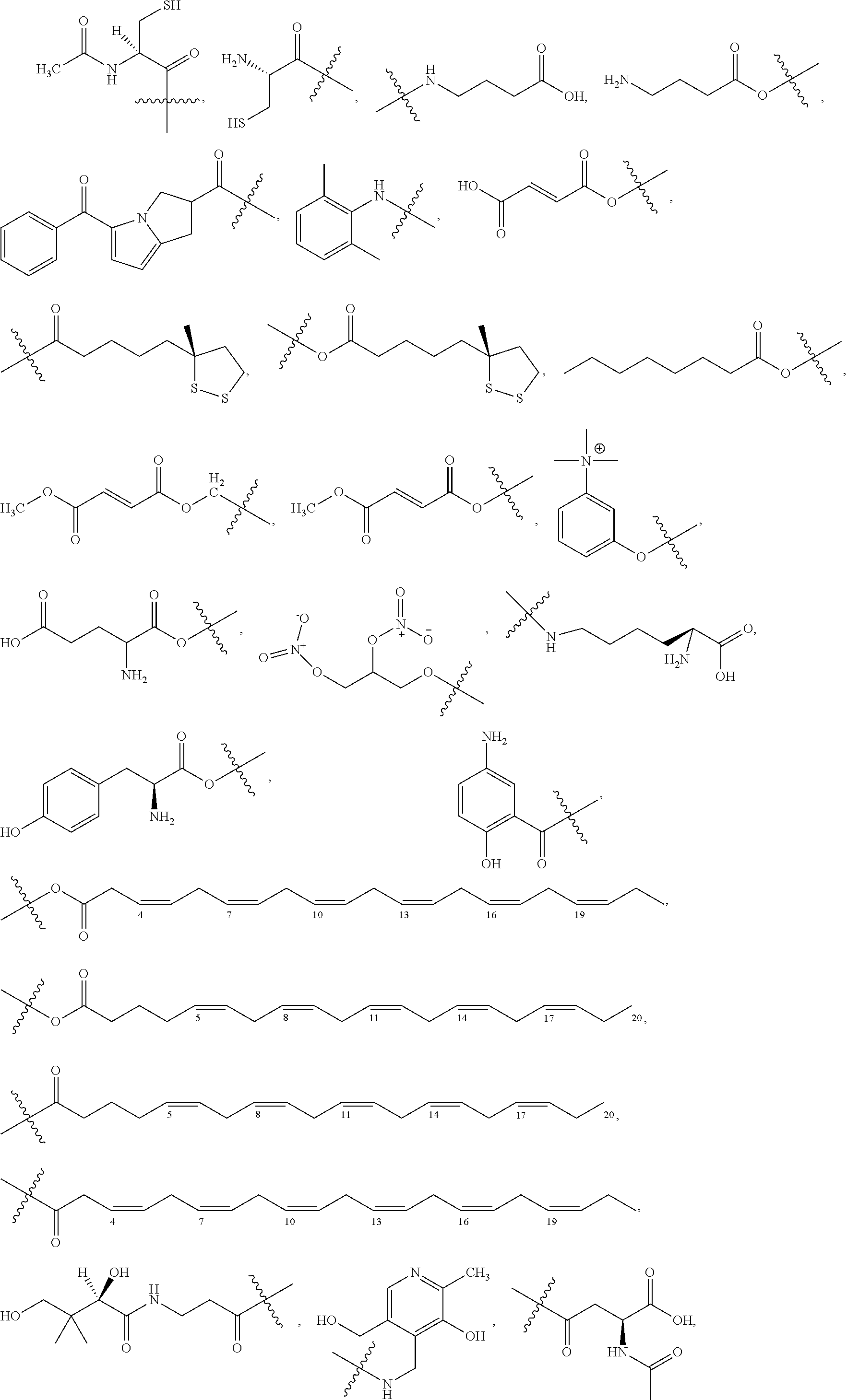

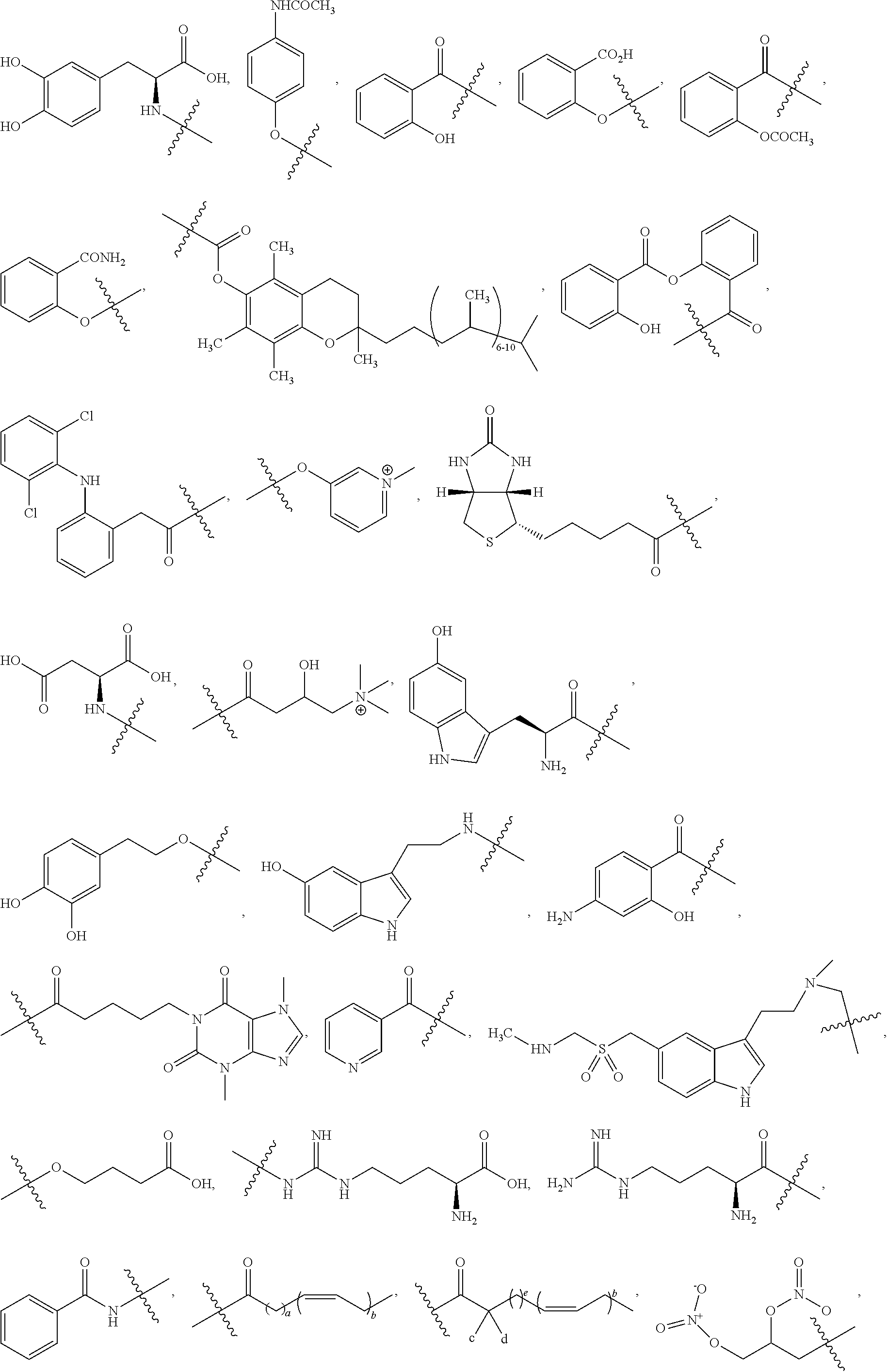

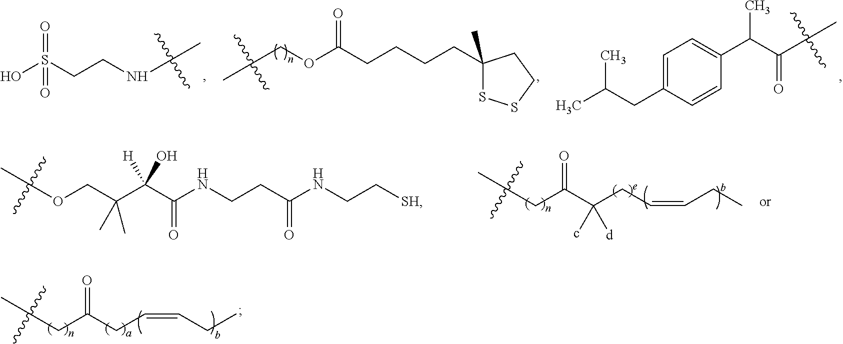

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;
   R³ represents H, D, CH₃CO, CD₃CO, CD₃, CH₃,

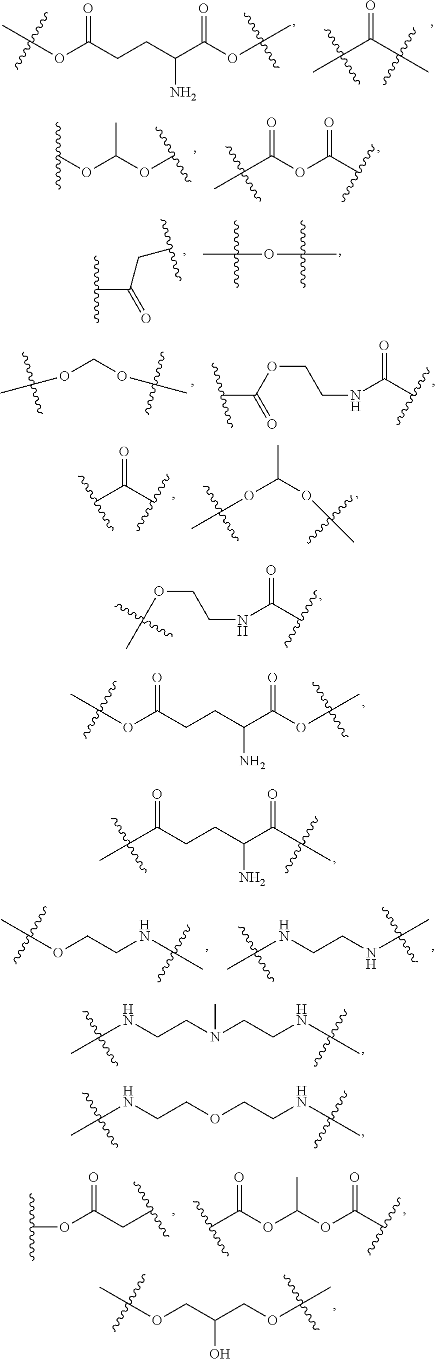

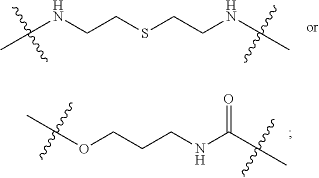 [SMILES: *NCCSCCN* ; *OCCCNC(*)=O]

R⁴ represents D, CH₃CO, CD₃CO, CD₃,

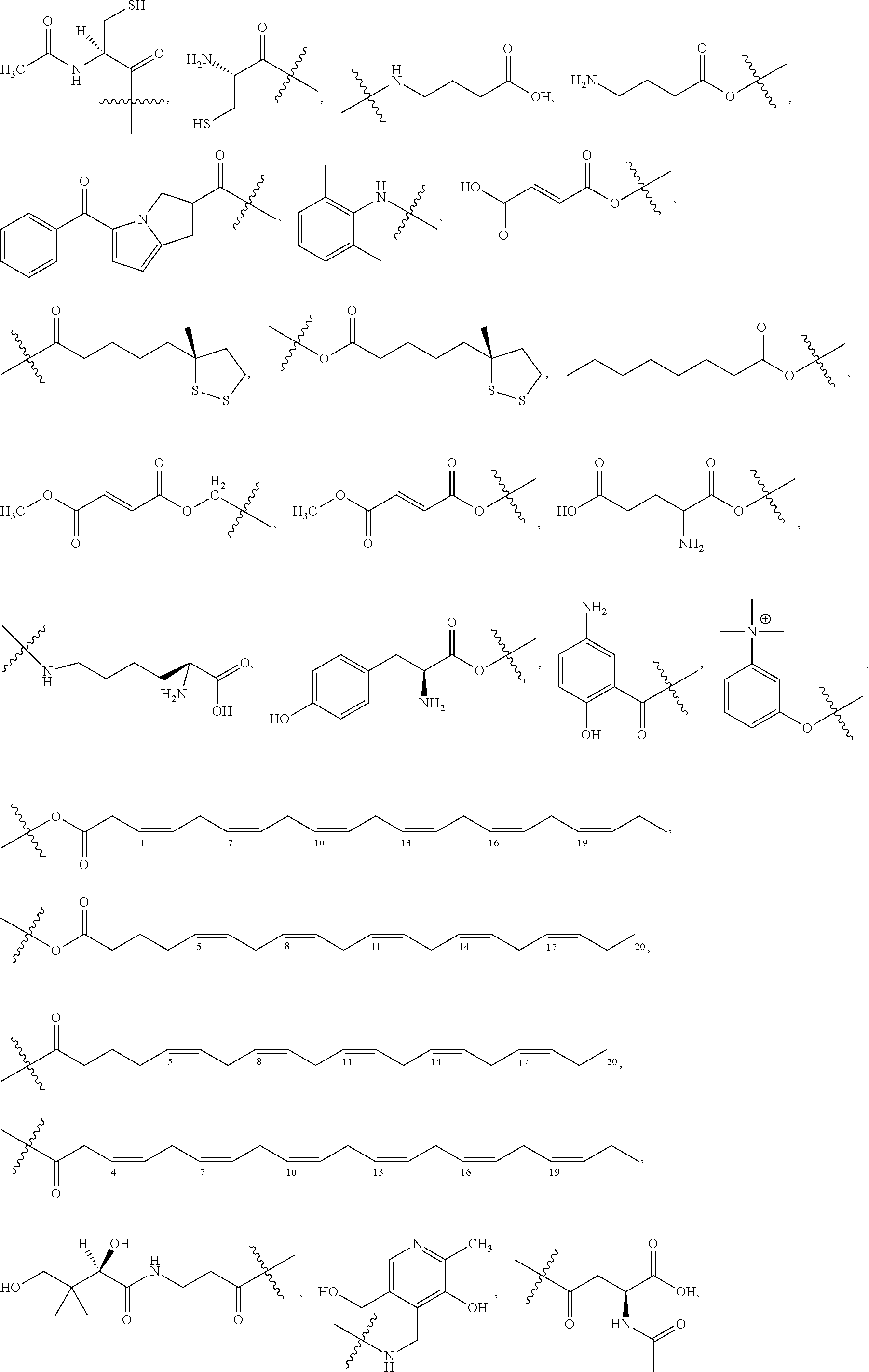

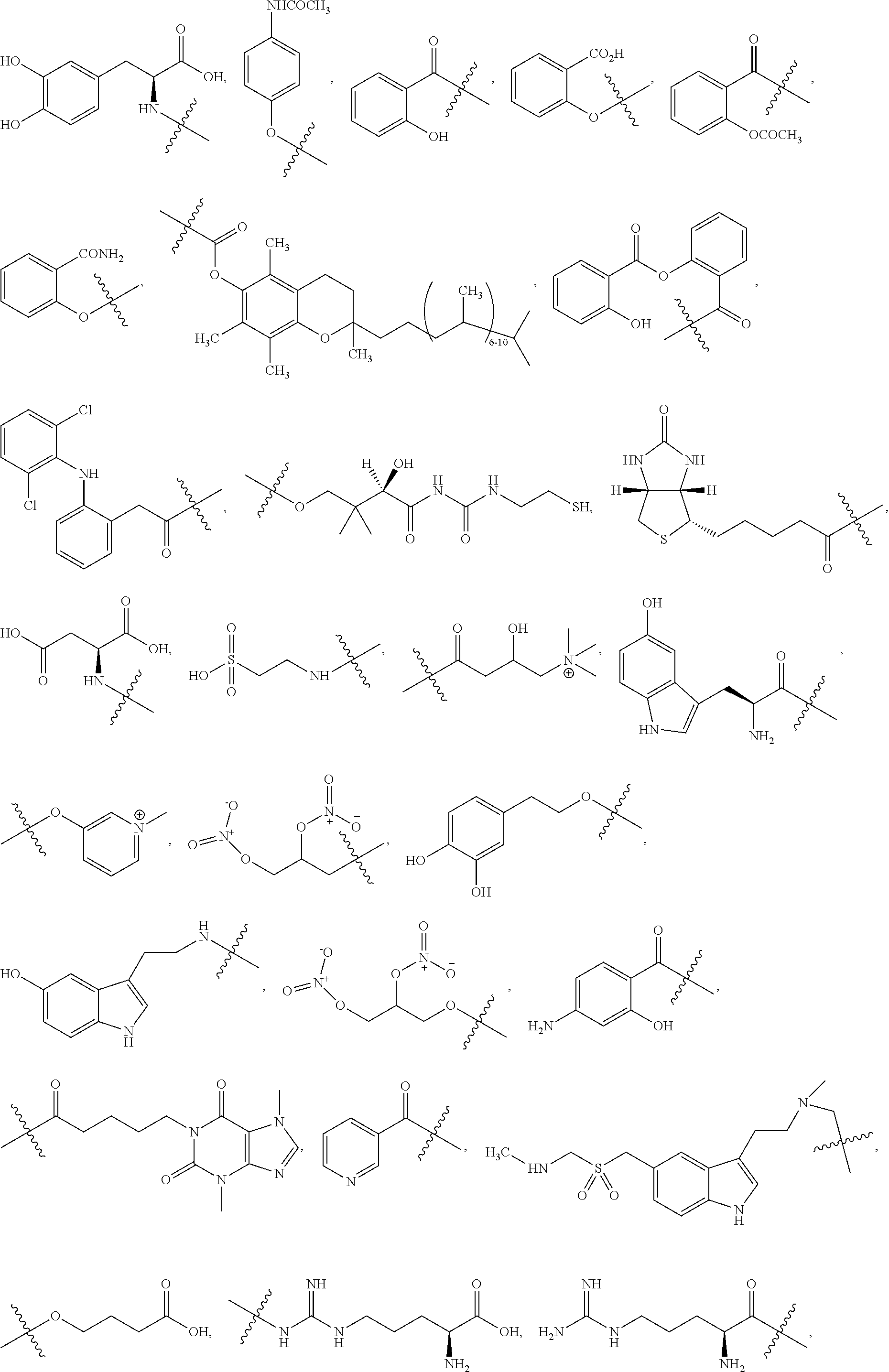

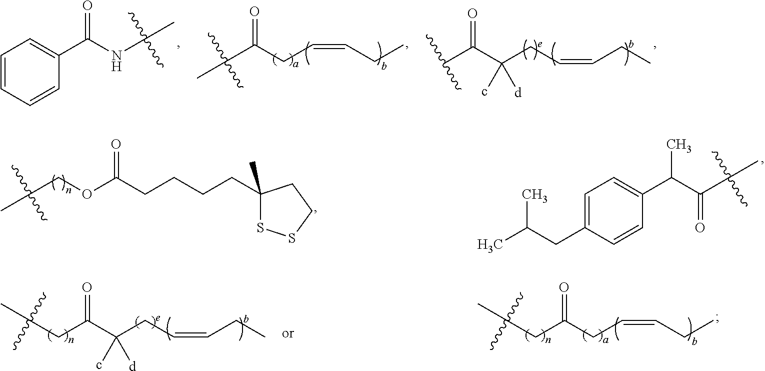

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;

In the illustrative embodiments, examples of compounds of formula I, formula II and Formula III are as set forth below:

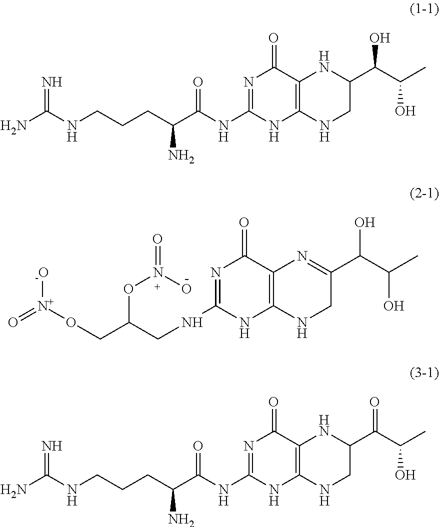

Herein the application also provides a kit comprising any of the pharmaceutical compositions disclosed herein. The kit may comprise instructions for use in the treatment of metabolic diseases or its related complications.

The application also discloses a pharmaceutical composition comprising a pharmaceutically acceptable carrier and any of the compositions herein. In some aspects, the pharmaceutical composition is formulated for systemic administration, oral administration, sustained release, parenteral administration, injection, subdermal administration, or transdermal administration.

Herein, the application additionally provides kits comprising the pharmaceutical compositions described herein. The kits may further comprise instructions for use in the treatment of metabolic diseases or its related complications.

The compositions described herein have several uses. The present application provides, for example, methods of treating a patient suffering from metabolic diseases or its related complications manifested from metabolic or genetic conditions or disorders, metabolic diseases, chronic diseases or disorders; neurodegenerative disorders, Hepatology, Cancer, Respiratory, Hematological, Orthopedic, Cardiovascular, Renal, Skin, Vascular or Ocular complications.

### DETAILED DESCRIPTION OF THE INVENTION

### Definitions

As used herein, the following terms and phrases shall have the meanings set forth below. Unless defined otherwise, all technical and scientific terms used herein have the same meaning as commonly understood to one of ordinary skill in the art.

The compounds of the present invention can be present in the form of pharmaceutically acceptable salts. The compounds of the present invention can also be present in the form of pharmaceutically acceptable esters (i.e., the methyl and ethyl esters of the acids of formula I, formula II and formula III to be used as prodrugs). The compounds of the present invention can also be solvated, i.e. hydrated. The solvation can be affected in the course of the manufacturing process or can take place i.e. as a consequence of hygroscopic properties of an initially anhydrous compound of formula I, formula II and formula III (hydration).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, lngold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

As used herein, the term “metabolic condition” refers to an Inborn errors of metabolism (or genetic metabolic conditions) are genetic disorders that result from a defect in one or more metabolic pathways; specifically, the function of an enzyme is affected and is either deficient or completely absent.

The term “polymorph” as used herein is art-recognized and refers to one crystal structure of a given compound.

The phrases “parenteral administration” and “administered parenterally” as used herein refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra-articular, subcapsular, subarachnoid, intraspinal and intrastemal injection and infusion.

A “patient,” “subject,” or “host” to be treated by the subject method may mean either a human or non-human animal, such as primates, mammals, and vertebrates.

The phrase “pharmaceutically acceptable” is art-recognized. In certain embodiments, the term includes compositions, polymers and other materials and/or dosage forms which are, within the scope of sound medical judgment, suitable for use in contact with the tissues of mammals, human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “predicting” as used herein refers to assessing the probability related diseases patient will suffer from abnormalities or complication and/or terminal platelet aggregation or failure and/or death (i.e. mortality) within a defined time window (predictive window) in the future. The mortality may be caused by the central nervous system or complication. The predictive window is an interval in which the subject will develop one or more of the said complications according to the predicted probability. The predictive window may be the entire remaining lifespan of the subject upon analysis by the method of the present invention.

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating phenylketonuria, cardiovascular disease, autism, ADHD, hypertension, endothelial dysfunction, and chronic kidney disease of a subject by administration of an agent even though such agent does not treat the cause of the condition. The term “treating”, “treat” or “treatment” as used herein includes curative, preventative (e.g., prophylactic), adjunct and palliative treatment.

The phrase “therapeutically effective amount” is an art-recognized term. In certain embodiments, the term refers to an amount of a salt or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

In certain embodiments, the pharmaceutical compositions described herein are formulated in a manner such that said compositions will be delivered to a patient in a therapeutically effective amount, as part of a prophylactic or therapeutic treatment. The desired amount of the composition to be administered to a patient will depend on absorption, inactivation, and excretion rates of the drug as well as the delivery rate of the salts and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular salt or composition may be adjusted to accommodate variations in the treatment parameters. Such treatment parameters include the clinical use to which the preparation is put, e.g., the site treated, the type of patient, e.g., human or non-human, adult or child, and the nature of the disease or condition.

In certain embodiments, the dosage of the subject compositions provided herein may be determined by reference to the plasma concentrations of the therapeutic composition or other encapsulated materials. For example, the maximum plasma concentration (Cmax) and the area under the plasma concentration-time curve from time 0 to infinity may be used.

When used with respect to a pharmaceutical composition or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., an therapeutic and/or biologically active salt and/or composition, for a sustained or extended period (as compared to the release from a bolus). This release may result in prolonged delivery of therapeutically effective amounts of any of the therapeutic agents disclosed herein.

The phrases “systemic administration,” “administered systemically,” “peripheral administration” and “administered peripherally” are art-recognized, and include the administration of a subject composition, therapeutic or other material at a site remote from the disease being treated. Administration of an agent for the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of a salt or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

The present disclosure also contemplates prodrugs of the compositions disclosed herein, as well as pharmaceutically acceptable salts of said prodrugs.

This application also discloses a pharmaceutical composition comprising a pharmaceutically acceptable carrier and the composition of a compound of Formula I, formula II or formula III may be formulated for systemic or topical or oral administration. The pharmaceutical composition may be also formulated for oral administration, oral solution, injection, subdermal administration, or transdermal administration. The pharmaceutical composition may further comprise at least one of a pharmaceutically acceptable stabilizer, diluent, surfactant, filler, binder, and lubricant.

In many embodiments, the pharmaceutical compositions described herein will incorporate the disclosed compounds and compositions (Formula I, formula II and formula III) to be delivered in an amount sufficient to deliver to a patient a therapeutically effective amount of a compound of formula I, formula II or formula III or composition as part of a prophylactic or therapeutic treatment. The desired concentration of formula I, formula II or formula III or its pharmaceutical acceptable salts will depend on absorption, inactivation, and excretion rates of the drug as well as the delivery rate of the salts and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular compound of formula I, formula II or formula III may be adjusted to accommodate variations in the treatment parameters. Such treatment parameters include the clinical use to which the preparation is put, e.g., the site treated, the type of patient, e.g., human or non-human, adult or child, and the nature of the disease or condition.

The concentration and/or amount of any compound of formula I, formula II or formula III may be readily identified by routine screening in animals, e.g., rats, by screening a range of concentration and/or amounts of the material in question using appropriate assays. Known methods are also available to assay local tissue concentrations, diffusion rates of the salts or compositions, and local blood flow before and after administration of therapeutic formulations disclosed herein. One such method is microdialysis, as reviewed by T. E. Robinson et al., 1991, microdialysis in the neurosciences, Techniques, volume 7, Chapter 1. The methods reviewed by Robinson may be applied, in brief, as follows. A microdialysis loop is placed in situ in a test animal. Dialysis fluid is pumped through the loop. When compounds with formula I, formula II or formula III such as those disclosed herein are injected adjacent to the loop, released drugs are collected in the dialysate in proportion to their local tissue concentrations. The progress of diffusion of the salts or compositions may be determined thereby with suitable calibration procedures using known concentrations of salts or compositions.

In certain embodiments, the dosage of the subject compounds of formula I, formula II or formula III provided herein may be determined by reference to the plasma concentrations of the therapeutic composition or other encapsulated materials. For example, the maximum plasma concentration (Cmax) and the area under the plasma concentration-time curve from time 0 to infinity may be used.

Generally, in carrying out the methods detailed in this application, an effective dosage for the compounds of Formulas I is in the range of about 0.01 mg/kg/day to about 100 mg/kg/day in single or divided doses, for instance 0.01 mg/kg/day to about 50 mg/kg/day in single or divided doses. The compounds of Formulas I may be administered at a dose of, for example, less than 0.2 mg/kg/day, 0.5 mg/kg/day, 1.0 mg/kg/day, 5 mg/kg/day, 10 mg/kg/day, 20 mg/kg/day, 30 mg/kg/day, or 40 mg/kg/day. Compounds of Formula I, formula II or formula III may also be administered to a human patient at a dose of, for example, between 0.1 mg and 1000 mg, between 5 mg and 80 mg, or less than 1.0, 9.0, 12.0, 20.0, 50.0, 75.0, 100, 300, 400, 500, 800, 1000, 2000, 5000 mg per day. In certain embodiments, the compositions herein are administered at an amount that is less than 95%, 90%, 80%, 70%, 60%, 50%, 40%, 30%, 20%, or 10% of the compound of formula I, formula II or formula III required for the same therapeutic benefit.

An effective amount of the compounds of formula I, formula II or formula III described herein refers to the amount of one of said salts or compositions which is capable of inhibiting or preventing a disease.

An effective amount may be sufficient to prohibit, treat, alleviate, ameliorate, halt, restrain, slow or reverse the progression, or reduce the severity of a complication resulting from nerve damage or demyelization and/or elevated reactive oxidative-nitrosative species and/or abnormalities in neurotransmitter homeostasis's, in patients who are at risk for such complications. As such, these methods include both medical therapeutic (acute) and/or prophylactic (prevention) administration as appropriate. The amount and timing of compositions administered will, of course, be dependent on the subject being treated, on the severity of the affliction, on the manner of administration and on the judgment of the prescribing physician. Thus, because of patient-to-patient variability, the dosages given above are a guideline and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases.

The compositions provided by this application may be administered to a subject in need of treatment by a variety of conventional routes of administration, including orally, topically, parenterally, e.g., intravenously, subcutaneously or intramedullary. Further, the compositions may be administered intranasally, as a rectal suppository, or using a “flash” formulation, i.e., allowing the medication to dissolve in the mouth without the need to use water. Furthermore, the compositions may be administered to a subject in need of treatment by controlled release dosage forms, site specific drug delivery, transdermal drug delivery, patch (active/passive) mediated drug delivery, by stereotactic injection, or in nanoparticles.

The compositions may be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compositions and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like. Thus, for purposes of oral administration, tablets containing various excipients such as L-arginine, sodium citrate, calcium carbonate and calcium phosphate may be employed along with various disintegrates such as starch, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Appropriate materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof. The compounds of formula I, formula II or formula III may also comprise enterically coated comprising of various excipients, as is well known in the pharmaceutical art.

For parenteral administration, solutions of the compositions may be prepared in (for example) sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

The formulations, for instance tablets, may contain e.g. 10 to 100, 50 to 250, 150 to 500 mg, or 350 to 800 mg e.g. 10, 50, 100, 300, 500, 700, 800 mg of the compounds of formula I, formula II or formula III disclosed herein, for instance, compounds of formula I, formula II or formula III or pharmaceutical acceptable salts of a compounds of Formula I.

Generally, a composition as described herein may be administered orally, or parenterally (e.g., intravenous, intramuscular, subcutaneous or intramedullary). Topical administration may also be indicated, for example, where the patient is suffering from gastrointestinal disorder that prevent oral administration, or whenever the medication is best applied to the surface of a tissue or organ as determined by the attending physician. Localized administration may also be indicated, for example, when a high dose is desired at the target tissue or organ. For buccal administration the active composition may take the form of tablets or lozenges formulated in a conventional manner.

The dosage administered will be dependent upon the identity of the metabolic disease; the type of host involved, including its age, health and weight; the kind of concurrent treatment, if any; the frequency of treatment and therapeutic ratio.

Illustratively, dosage levels of the administered active ingredients are: intravenous, 0.1 to about 200 mg/kg; intramuscular, 1 to about 500 mg/kg; orally, 5 to about 1000 mg/kg; intranasal instillation, 5 to about 1000 mg/kg; and aerosol, 5 to about 1000 mg/kg of host body weight.

Expressed in terms of concentration, an active ingredient can be present in the compositions of the present invention for localized use about the cutis, intranasally, pharyngolaryngeally, bronchially, intravaginally, rectally, or ocularly in a concentration of from about 0.01 to about 50% w/w of the composition; preferably about 1 to about 20% w/w of the composition; and for parenteral use in a concentration of from about 0.05 to about 50% w/v of the composition and preferably from about 5 to about 20% w/v.

The compositions of the present invention are preferably presented for administration to humans and animals in unit dosage forms, such as tablets, capsules, pills, powders, granules, suppositories, sterile parenteral solutions or suspensions, sterile non-parenteral solutions of suspensions, and oral solutions or suspensions and the like, containing suitable quantities of an active ingredient. For oral administration either solid or fluid unit dosage forms can be prepared.

As discussed above, the tablet core contains one or more hydrophilic polymers. Suitable hydrophilic polymers include, but are not limited to, water swellable cellulose derivatives, polyalkylene glycols, thermoplastic polyalkylene oxides, acrylic polymers, hydrocolloids, clays, gelling starches, swelling cross-linked polymers, and mixtures thereof. Examples of suitable water swellable cellulose derivatives include, but are not limited to, sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, and hydroxypropylethylcellulose, and mixtures thereof. Examples of suitable polyalkylene glycols include, but are not limited to, polyethylene glycol. Examples of suitable thermoplastic polyalkylene oxides include, but are not limited to, poly(ethylene oxide). Examples of suitable acrylic polymers include, but are not limited to, potassium methacrylatedivinylbenzene copolymer, polymethylmethacrylate, high-molecular weight crosslinked acrylic acid homopolymers and copolymers such as those commercially available from Noveon Chemicals under the tradename CARBOPOL™. Examples of suitable hydrocolloids include, but are not limited to, alginates, agar, guar gum, locust bean gum, kappa carrageenan, iota carrageenan, tara, gum arabic, tragacanth, pectin, xanthan gum, gellan gum, maltodextrin, galactomannan, pusstulan, laminarin, scleroglucan, gum arabic, inulin, pectin, gelatin, whelan, rhamsan, zooglan, methylan, chitin, cyclodextrin, chitosan, and mixtures thereof. Examples of suitable clays include, but are not limited to, smectites such as bentonite, kaolin, and laponite; magnesium trisilicate; magnesium aluminum silicate; and mixtures thereof. Examples of suitable gelling starches include, but are not limited to, acid hydrolyzed starches, swelling starches such as sodium starch glycolate and derivatives thereof, and mixtures thereof. Examples of suitable swelling cross-linked polymers include, but are not limited to, cross-linked polyvinyl pyrrolidone, cross-linked agar, and cross-linked carboxymethylcellulose sodium, and mixtures thereof.

The carrier may contain one or more suitable excipients for the formulation of tablets. Examples of suitable excipients include, but are not limited to, fillers, adsorbents, binders, disintegrants, lubricants, glidants, release-modifying excipients, superdisintegrants, antioxidants, and mixtures thereof.

Suitable binders include, but are not limited to, dry binders such as polyvinyl pyrrolidone and hydroxypropylmethylcellulose; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pus stulan, laminarin, scleroglucan, inulin, whelan, rhamsan, zooglan, methylan, chitin, cyclodextrin, chitosan, polyvinyl pyrrolidone, cellulosics, sucrose, and starches; and mixtures thereof. Suitable disintegrants include, but are not limited to, sodium starch glycolate, cross-linked polyvinylpyrrolidone, cross-linked carboxymethylcellulose, starches, microcrystalline cellulose, and mixtures thereof.

Suitable lubricants include, but are not limited to, long chain fatty acids and their salts, such as magnesium stearate and stearic acid, talc, glycerides waxes, and mixtures thereof. Suitable glidants include, but are not limited to, colloidal silicon dioxide. Suitable release-modifying excipients include, but are not limited to, insoluble edible materials, pH-dependent polymers, and mixtures thereof.

Suitable insoluble edible materials for use as release-modifying excipients include, but are not limited to, water-insoluble polymers and low-melting hydrophobic materials, copolymers thereof, and mixtures thereof. Examples of suitable water-insoluble polymers include, but are not limited to, ethylcellulose, polyvinyl alcohols, polyvinyl acetate, polycaprolactones, cellulose acetate and its derivatives, acrylates, methacrylates, acrylic acid copolymers, copolymers thereof, and mixtures thereof. Suitable low-melting hydrophobic materials include, but are not limited to, fats, fatty acid esters, phospholipids, waxes, and mixtures thereof. Examples of suitable fats include, but are not limited to, hydrogenated vegetable oils such as for example cocoa butter, hydrogenated palm kernel oil, hydrogenated cottonseed oil, hydrogenated sunflower oil, and hydrogenated soybean oil, free fatty acids and their salts, and mixtures thereof. Examples of suitable fatty acid esters include, but are not limited to, sucrose fatty acid esters, mono-, di-, and triglycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, stearoyl macrogol-32 glycerides, and mixtures thereof. Examples of suitable phospholipids include phosphotidyl choline, phosphotidyl serene, phosphotidyl enositol, phosphotidic acid, and mixtures thereof. Examples of suitable waxes include, but are not limited to, carnauba wax, spermaceti wax, beeswax, candelilla wax, shellac wax, microcrystalline wax, and paraffin wax; fat-containing mixtures such as chocolate, and mixtures thereof. Examples of super disintegrants include, but are not limited to, croscarmellose sodium, sodium starch glycolate and cross-linked povidone (crospovidone). In one embodiment the tablet core contains up to about 5 percent by weight of such super disintegrant.

Examples of antioxidants include, but are not limited to, tocopherols, ascorbic acid, sodium pyrosulfite, butylhydroxytoluene, butylated hydroxyanisole, edetic acid, and edetate salts, and mixtures thereof. Examples of preservatives include, but are not limited to, citric acid, tartaric acid, lactic acid, malic acid, acetic acid, benzoic acid, and sorbic acid, and mixtures thereof.

In one embodiment, the immediate release coating has an average thickness of at least 50 microns, such as from about 50 microns to about 2500 microns; e.g., from about 250 microns to about 1000 microns. In embodiment, the immediate release coating is typically compressed at a density of more than about 0.9 g/cc, as measured by the weight and volume of that specific layer.

In one embodiment, the immediate release coating contains a first portion and a second portion, wherein at least one of the portions contains the second pharmaceutically active agent. In one embodiment, the portions contact each other at a center axis of the tablet. In one embodiment, the first portion includes the first pharmaceutically active agent and the second portion includes the second pharmaceutically active agent.

In one embodiment, the first portion contains the first pharmaceutically active agent and the second portion contains the second pharmaceutically active agent. In one embodiment, one of the portions contains a third pharmaceutically active agent. In one embodiment one of the portions contains a second immediate release portion of the same pharmaceutically active agent as that contained in the tablet core.

In one embodiment, the outer coating portion is prepared as a dry blend of materials prior to addition to the coated tablet core. In another embodiment the outer coating portion is included of a dried granulation including the pharmaceutically active agent.

Formulations with different drug release mechanisms described above could be combined in a final dosage form containing single or multiple units. Examples of multiple units include multilayer tablets, capsules containing tablets, beads, or granules in a solid or liquid form. Typical, immediate release formulations include compressed tablets, gels, films, coatings, liquids and particles that can be encapsulated, for example, in a gelatin capsule. Many methods for preparing coatings, covering or incorporating drugs, are known in the art.

The immediate release dosage, unit of the dosage form, i.e., a tablet, a plurality of drug-containing beads, granules or particles, or an outer layer of a coated core dosage form, contains a therapeutically effective quantity of the active agent with conventional pharmaceutical excipients. The immediate release dosage unit may or may not be coated, and may or may not be admixed with the delayed release dosage unit or units (as in an encapsulated mixture of immediate release drug-containing granules, particles or beads and delayed release drug-containing granules or beads).

Extended release formulations are generally prepared as diffusion or osmotic systems, for example, as described in “Remington—The Science and Practice of Pharmacy”, 20th. Ed., Lippincott Williams & Wilkins, Baltimore, Md., 2000). A diffusion system typically consists of one of two types of devices, reservoir and matrix, which are wellknown and described in die art. The matrix devices are generally prepared by compressing the drug with a slowly dissolving polymer carrier into a tablet form.

An immediate release portion can be added to the extended release system by means of either applying an immediate release layer on top of the extended release core; using coating or compression processes or in a multiple unit system such as a capsule containing extended and immediate release beads.

Delayed release dosage formulations are created by coating a solid dosage form with a film of a polymer which is insoluble in the acid environment of the stomach, but soluble in the neutral environment of small intestines. The delayed release dosage units can be prepared, for example, by coating a drug or a drug-containing composition with a selected coating material. The drug-containing composition may be a tablet for incorporation into a capsule, a tablet for use as an inner core in a “coated core” dosage form, or a plurality of drug-containing beads, particles or granules, for incorporation into either a tablet or capsule.

A pulsed release dosage form is one that mimics a multiple dosing profile without repeated dosing and typically allows at least a twofold reduction in dosing frequency as compared to the drug presented as a conventional dosage form (e.g., as a solution or prompt drug-releasing, conventional solid dosage form). A pulsed release profile is characterized by a time period of no release (lag time) or reduced release followed by rapid drug release.

Each dosage form contains a therapeutically effective amount of active agent. In one embodiment of dosage forms that mimic a twice daily dosing profile, approximately 30 wt. % to 70 wt. %, preferably 40 wt. % to 60 wt. %, of the total amount of active agent in the dosage form is released in the initial pulse, and, correspondingly approximately 70 wt. % to 3.0 wt. %, preferably 60 wt. % to 40 wt. %, of the total amount of active agent in the dosage form is released in the second pulse. For dosage forms mimicking the twice daily dosing profile, the second pulse is preferably released approximately 3 hours to less than 14 hours, and more preferably approximately 5 hours to 12 hours, following administration.

Another dosage form contains a compressed tablet or a capsule having a drug-containing immediate release dosage unit, a delayed release dosage unit and an optional second delayed release dosage unit. In this dosage form, the immediate release dosage unit contains a plurality of beads, granules particles that release drug substantially immediately following oral administration to provide an initial dose. The delayed release dosage unit contains a plurality of coated beads or granules, which release drug approximately 3 hours to 14 hours following oral administration to provide a second dose.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of one or more compounds of formula I, formula II or formula III or other active agents are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

In addition, in certain embodiments, subject compositions of the present application maybe lyophilized or subjected to another appropriate drying technique such as spray drying. The subject compositions may be administered once, or may be divided into a number of smaller doses to be administered at varying intervals of time, depending in part on the release rate of the compositions and the desired dosage.

Formulations useful in the methods provided herein include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of a subject composition which may be combined with a carrier material to produce a single dose may vary depending upon the subject being treated, and the particular mode of administration.

Methods of preparing these formulations or compositions include the step of bringing into association subject compositions with the carrier and, optionally, one or more accessory ingredients. In general, the formulations are prepared by uniformly and intimately bringing into association a subject composition with liquid carriers, or finely divided solid carriers, or both, and then, if necessary, shaping the product.

The compounds of formula I, formula II or formula III described herein may be administered in inhalant or aerosol formulations. The inhalant or aerosol formulations may comprise one or more agents, such as adjuvants, diagnostic agents, imaging agents, or therapeutic agents useful in inhalation therapy. The final aerosol formulation may for example contain 0.005-90% w/w, for instance 0.005-50%, 0.005-5% w/w, or 0.01-1.0% w/w, of medicament relative to the total weight of the formulation.

In solid dosage forms for oral administration (capsules, tablets, pills, dragees, powders, granules and the like), the subject composition is mixed with one or more pharmaceutically acceptable carriers and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using lactose or milk sugars, as well as high molecular weight polyethylene glycols and the like.

Liquid dosage forms for oral administration include pharmaceutically acceptable emulsions, microemulsions, solutions, suspensions, syrups and elixirs. In addition to the subject compositions, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, corn, peanut, sunflower, soybean, olive, castor, and sesame oils), glycerol, tetrahydrofuryl alcohol, polyethylene glycols and fatty acid esters of sorbitan, and mixtures thereof.

Suspensions, in addition to the subject compositions, may contain suspending agents such as, for example, ethoxylated isostearyl alcohols, polyoxyethylene sorbitol, and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the encapsulated compound(s) and composition(s). Formulations which are suitable for vaginal administration also include pessaries, tampons, creams, gels, pastes, foams, or spray formulations containing such carriers as are known in the art to be appropriate.

Dosage forms for transdermal administration include powders, sprays, ointments, pastes, creams, lotions, gels, solutions, patches, and inhalants. A subject composition may be mixed under sterile conditions with a pharmaceutically acceptable carrier, and with any preservatives, buffers, or propellants that may be required. For transdermal administration, the complexes may include lipophilic and hydrophilic groups to achieve the desired water solubility and transport properties.

The ointments, pastes, creams and gels may contain, in addition to subject compositions, other carriers, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays may contain, in addition to a subject composition, excipients such as lactose, talc, silicic acid, aluminum hydroxide, calcium silicates and polyamide powder, or mixtures of such substances. Sprays may additionally contain customary propellants, such as chlorofluorohydrocarbons and volatile unsubstituted hydrocarbons, such as butane and propane.

Methods of delivering a composition or compositions via a transdermal patch are known in the art. Exemplary patches and methods of patch delivery are described in U.S. Pat. Nos. 6,974,588, 6,564,093, 6,312,716, 6,440,454, 6,267,983, 6,239,180, and 6,103,275.

In another embodiment, a transdermal patch may comprise: a substrate sheet comprising a composite film formed of a resin composition comprising 100 parts by weight of a polyvinyl chloride-polyurethane composite and 2-10 parts by weight of a styrene-ethylene-butylene-styrene copolymer, a first adhesive layer on the one side of the composite film, and a polyalkylene terephthalate film adhered to the one side of the composite film by means of the first adhesive layer, a primer layer which comprises a saturated polyester resin and is formed on the surface of the polyalkylene terephthalate film; and a second adhesive layer comprising a styrene-diene-styrene block copolymer containing a pharmaceutical agent layered on the primer layer. A method for the manufacture of the above-mentioned substrate sheet comprises preparing the above resin composition molding the resin composition into a composite film by a calendar process, and then adhering a polyalkylene terephthalate film on one side of the composite film by means of an adhesive layer thereby forming the substrate sheet, and forming a primer layer comprising a saturated polyester resin on the outer surface of the polyalkylene terephthalate film.

Another type of patch comprises incorporating the drug directly in a pharmaceutically acceptable adhesive and laminating the drug-containing adhesive onto a suitable backing member, e.g. a polyester backing membrane. The drug should be present at a concentration which will not affect the adhesive properties, and at the same time deliver the required clinical dose.

Transdermal patches may be passive or active. Passive transdermal drug delivery systems currently available, such as the nicotine, estrogen and nitroglycerine patches, deliver small-molecule drugs. Many of the newly developed proteins and peptide drugs are too large to be delivered through passive transdermal patches and may be delivered using technology such as electrical assist (iontophoresis) for large-molecule drugs.

Iontophoresis is a technique employed for enhancing the flux of ionized substances through membranes by application of electric current. One example of an iontophoretic membrane is given in U.S. Pat. No. 5,080,646 to Theeuwes. The principal mechanisms by which iontophoresis enhances molecular transport across the skin are (a) repelling a charged ion from an electrode of the same charge, (b) electroosmosis, the convective movement of solvent that occurs through a charged pore in response the preferential passage of counter-ions when an electric field is applied or (c) increase skin permeability due to application of electrical current.

In some cases, it may be desirable to administer in the form of a kit, it may comprise a container for containing the separate compositions such as a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are widely used for the packaging of pharmaceutical unit dosage forms (tablets, capsules, and the like). Blister packs generally consist of a sheet of relatively stiff material covered with a foil of a plastic material that may be transparent.

Methods and compositions for the treatment of metabolic diseases. among other things, herein is provided a method of treating metabolic diseases, comprising administering to a patient in need thereof a therapeutically effective amount of compound of Formula I:

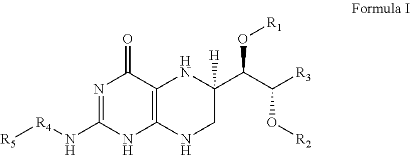

and pharmaceutically acceptable salts, hydrates, solvates, prodrugs, enantiomers, and stereoisomers thereof;

Wherein,
R¹, R², R³ each independently represents H, D, CH₃CO, CD₃CO, CD₃, CH₃,

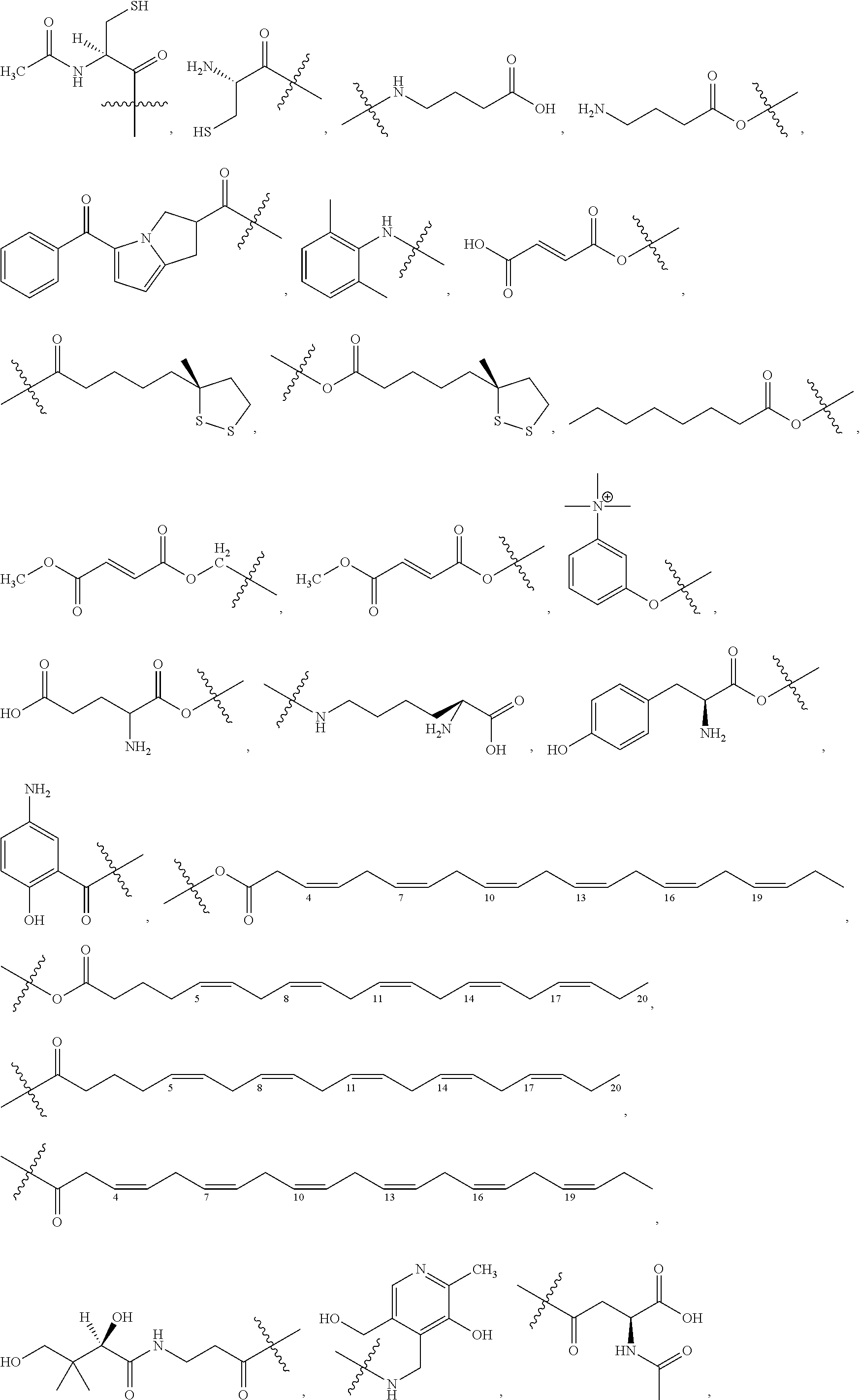

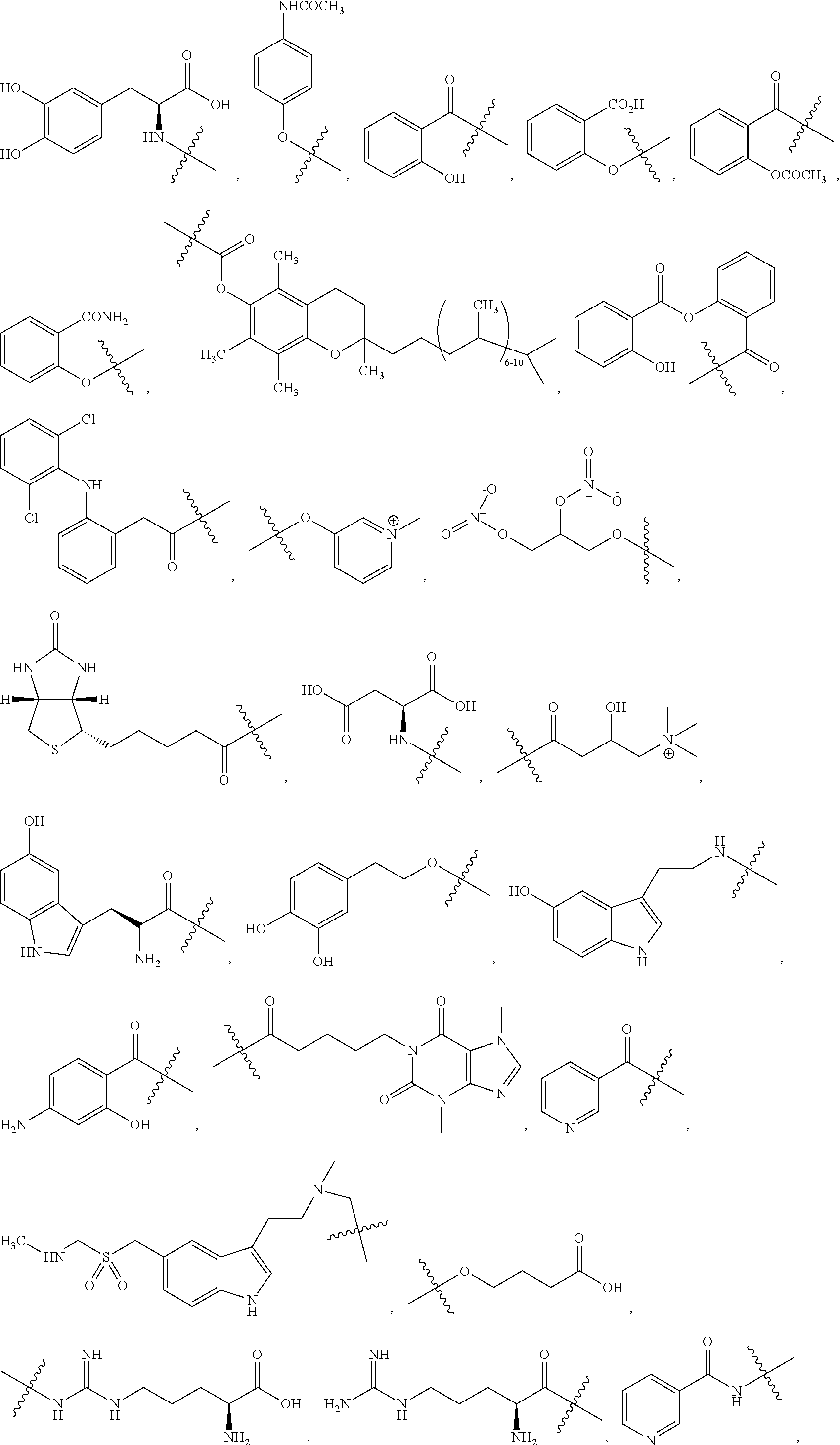

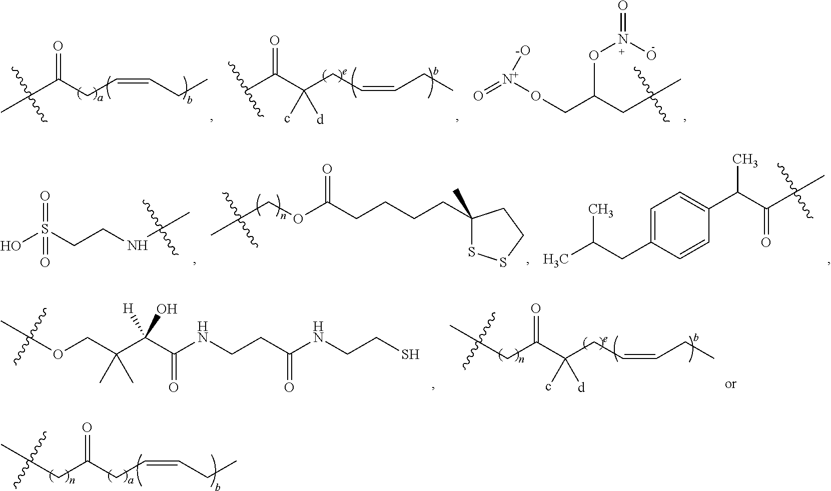

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;
   R⁴ represents H, D, CH₃CO, CD₃CO, CD₃, CH₃,

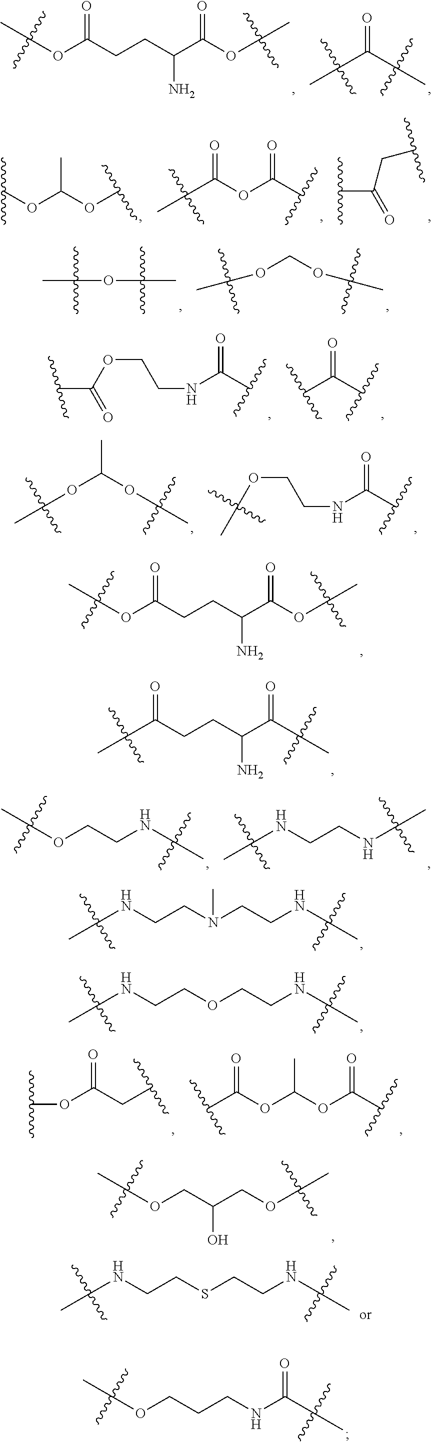

R⁵ represents D, CH₃CO, CD₃CO, CD₃,

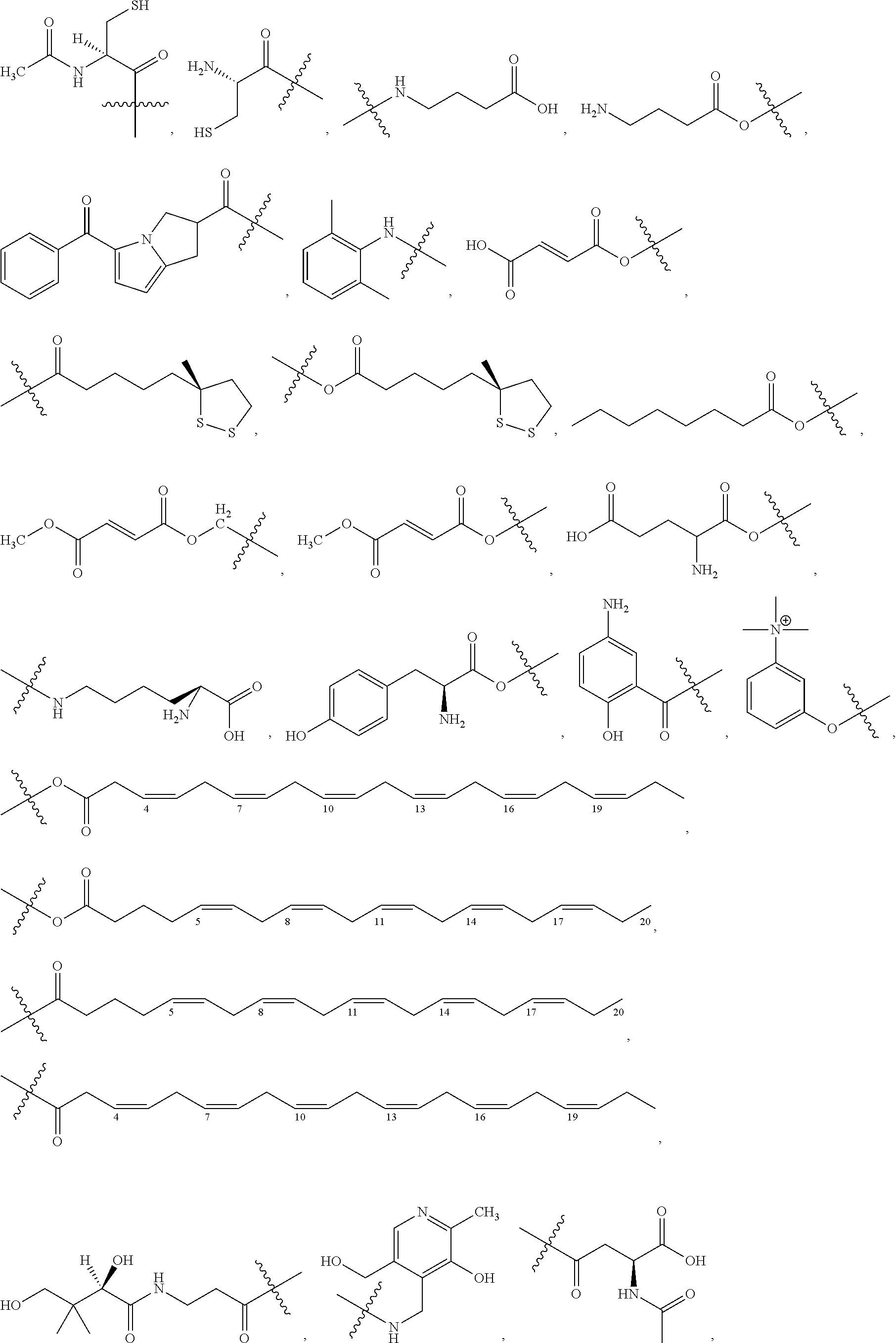

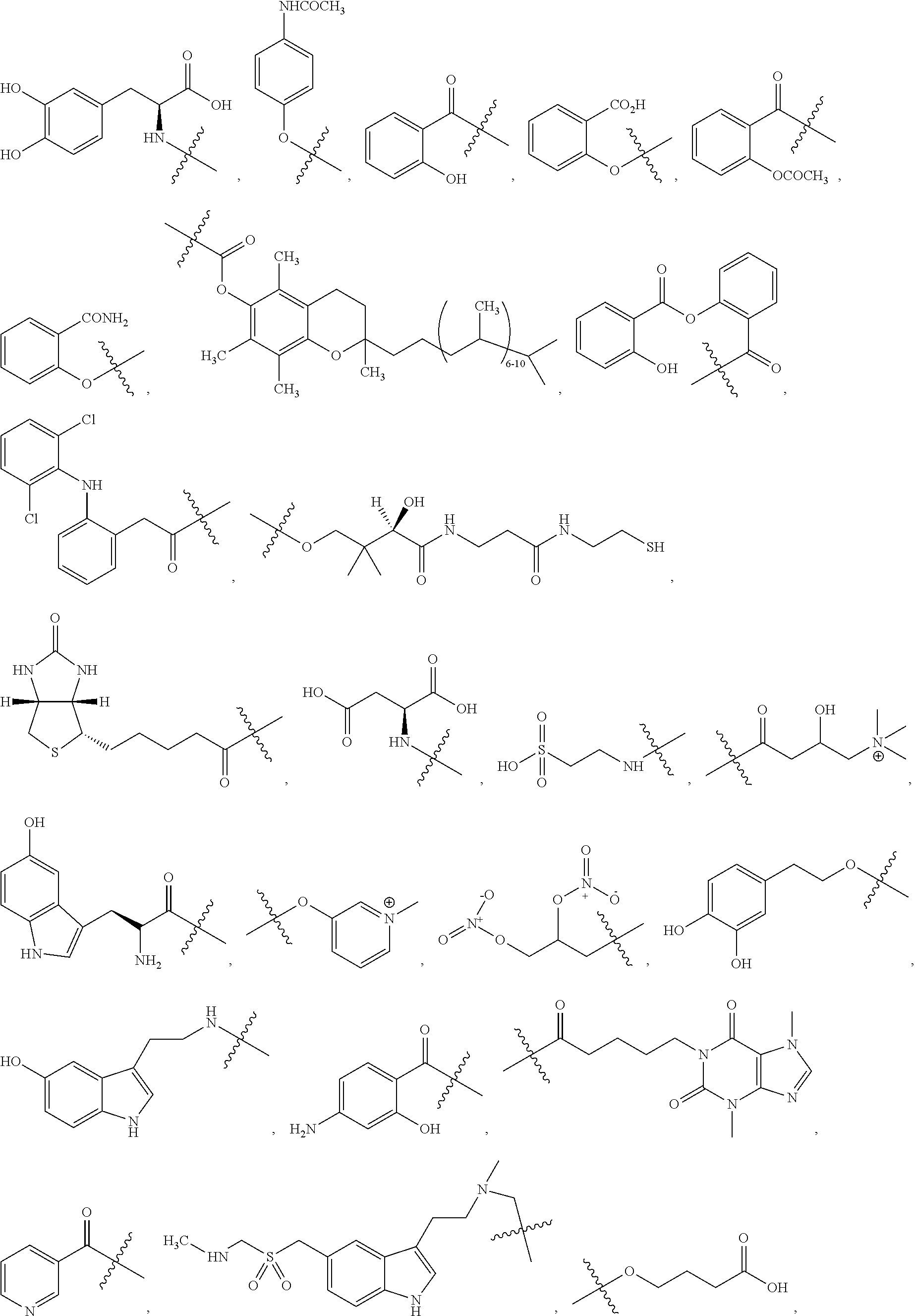

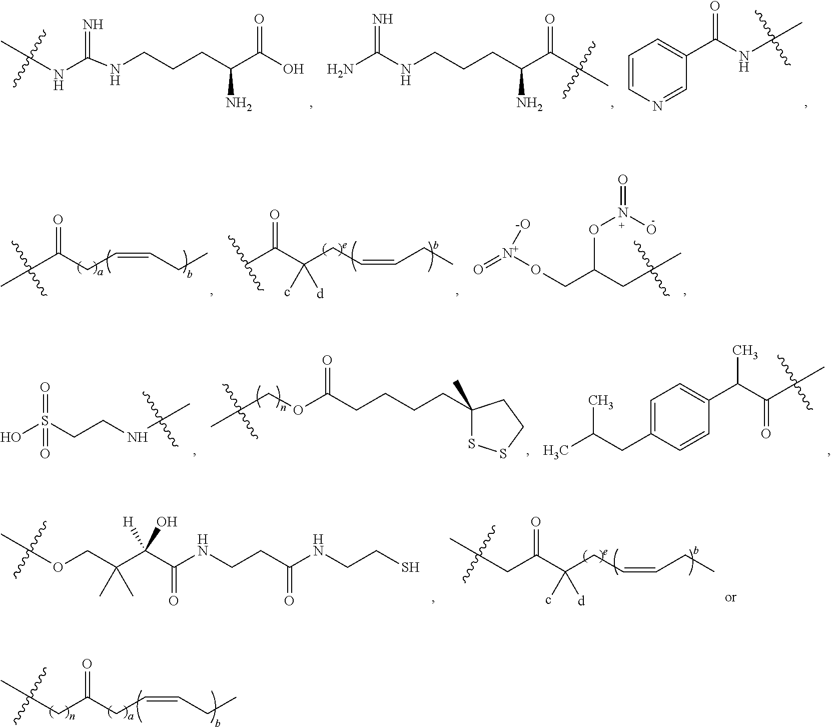

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;

Methods and compositions for the treatment of metabolic diseases. among other things, herein is provided a method of treating metabolic diseases, comprising administering to a patient in need thereof a therapeutically effective amount of compound of Formula II:

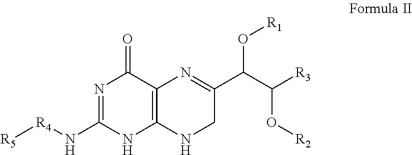

and pharmaceutically acceptable salts, hydrates, solvates, prodrugs, enantiomers, and stereoisomers thereof;

Wherein,
R¹, R², R³ each independently represents H, D, CH₃CO, CD₃CO, CD₃, CH₃,

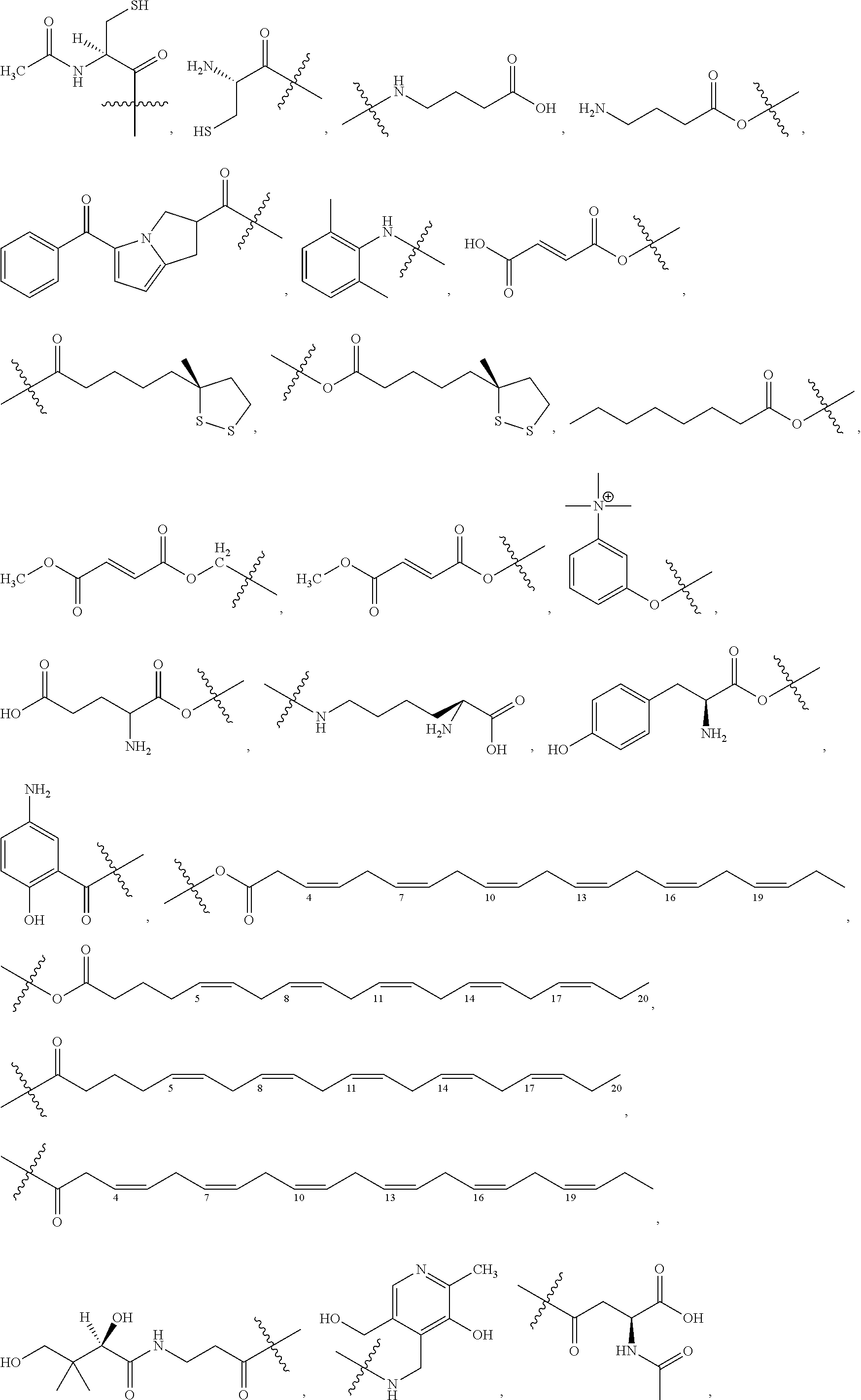

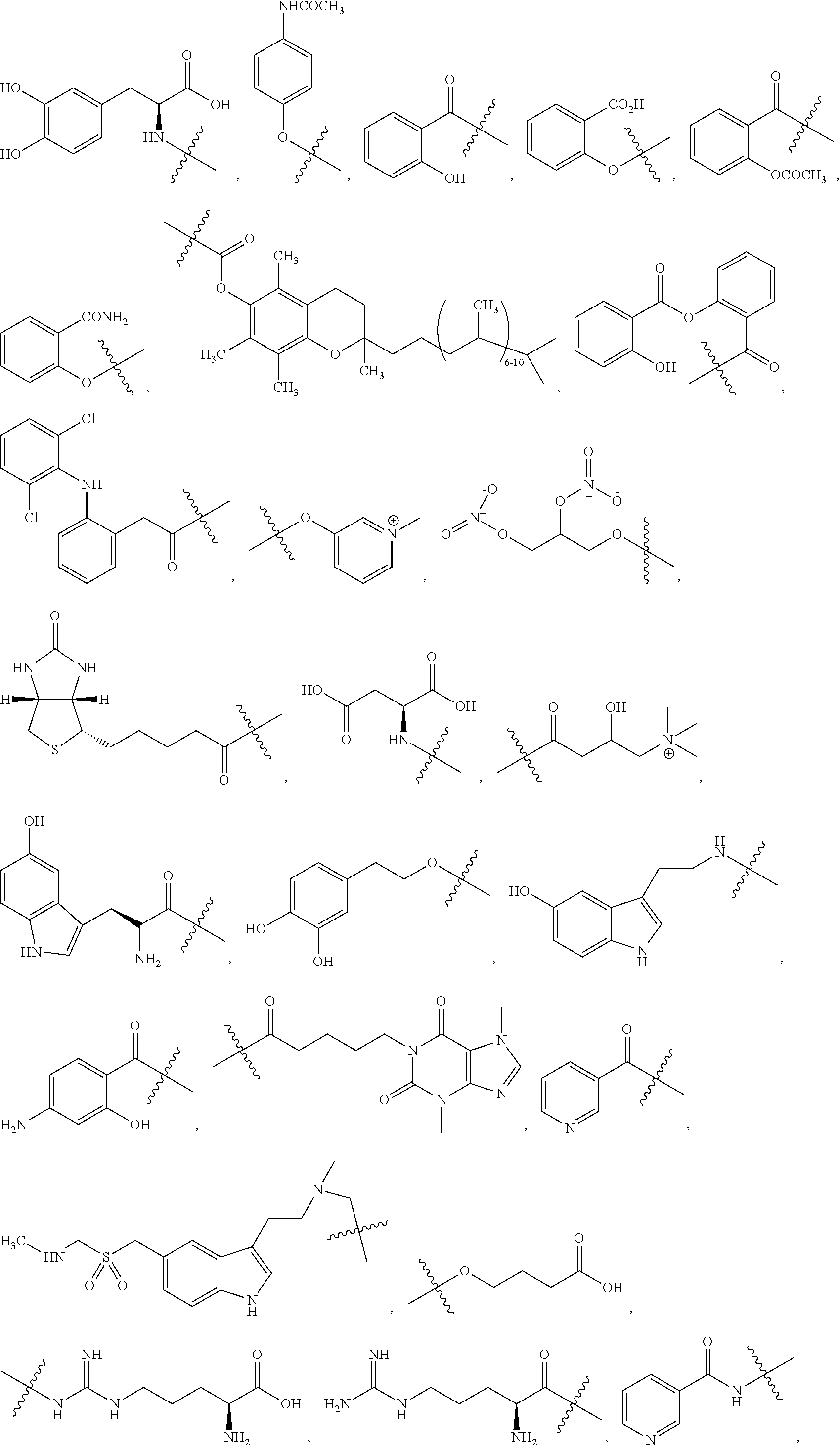

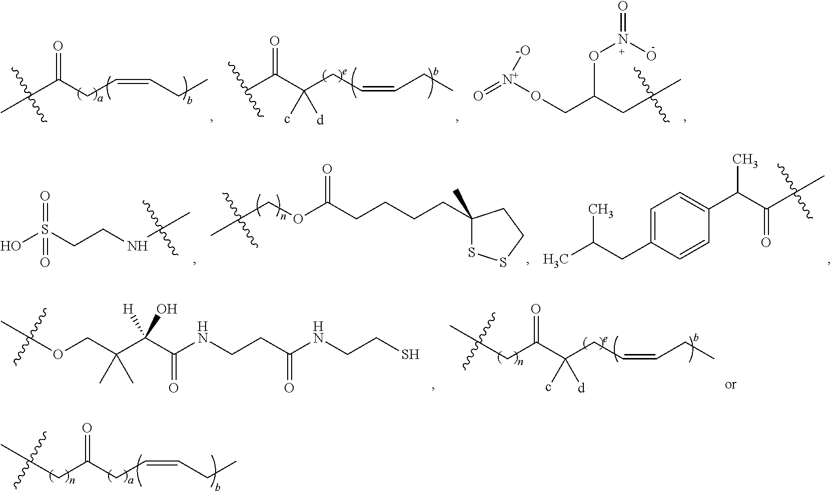

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;
   R⁴ represents H, D, CH₃CO, CD₃CO, CD₃, CH₃,

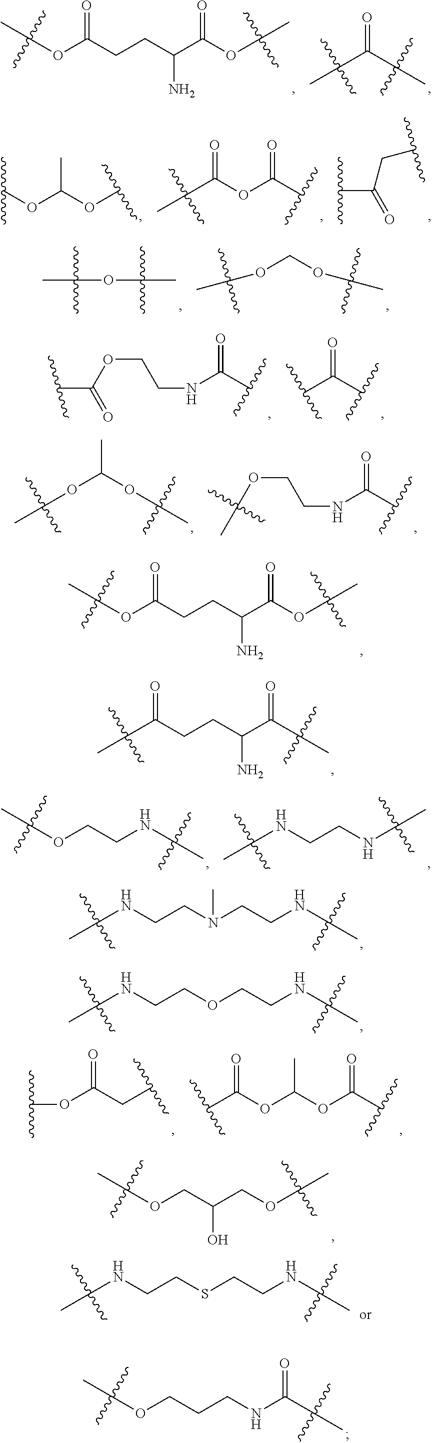

R⁵ represents D, CH₃CO, CD₃CO, CD₃,

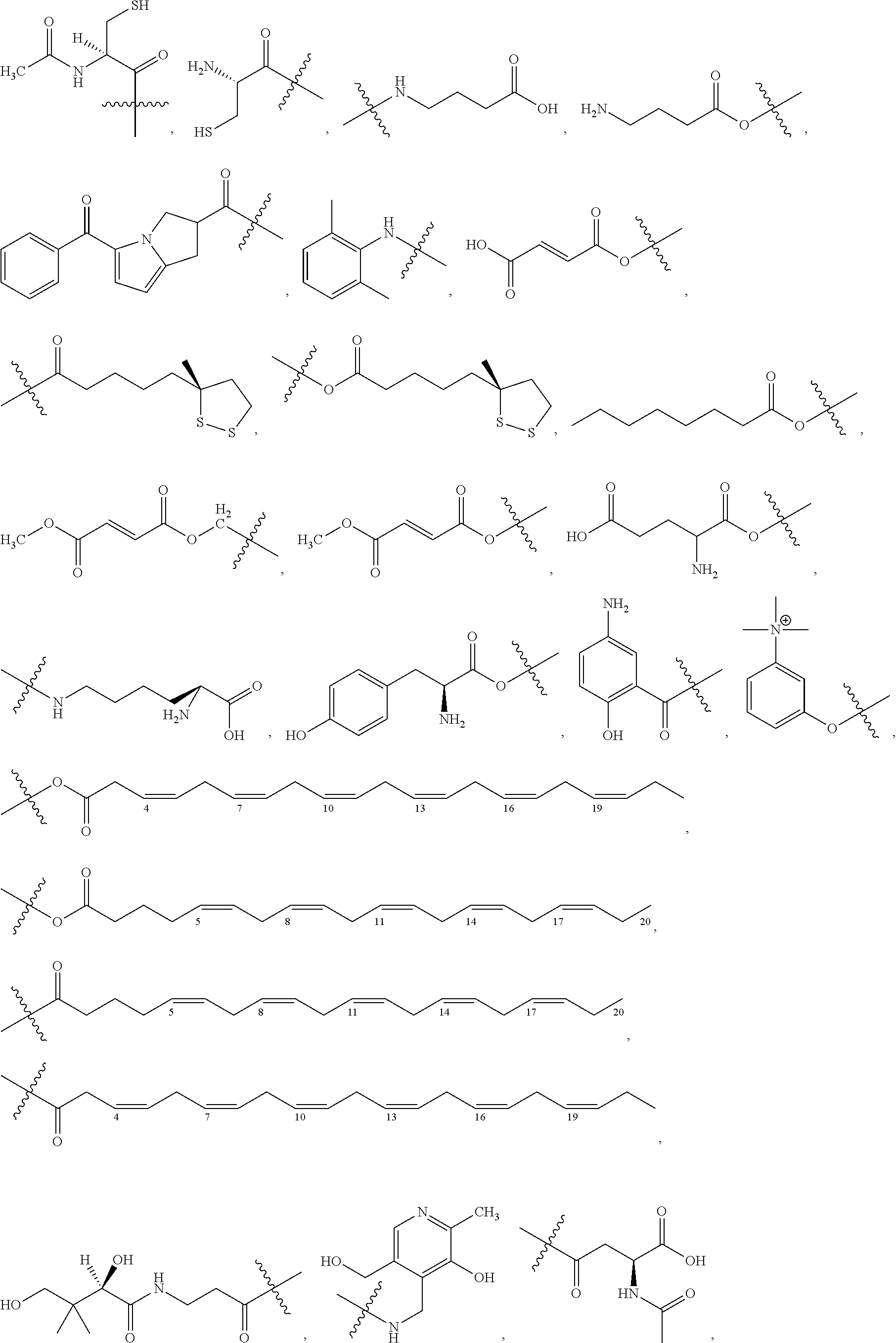

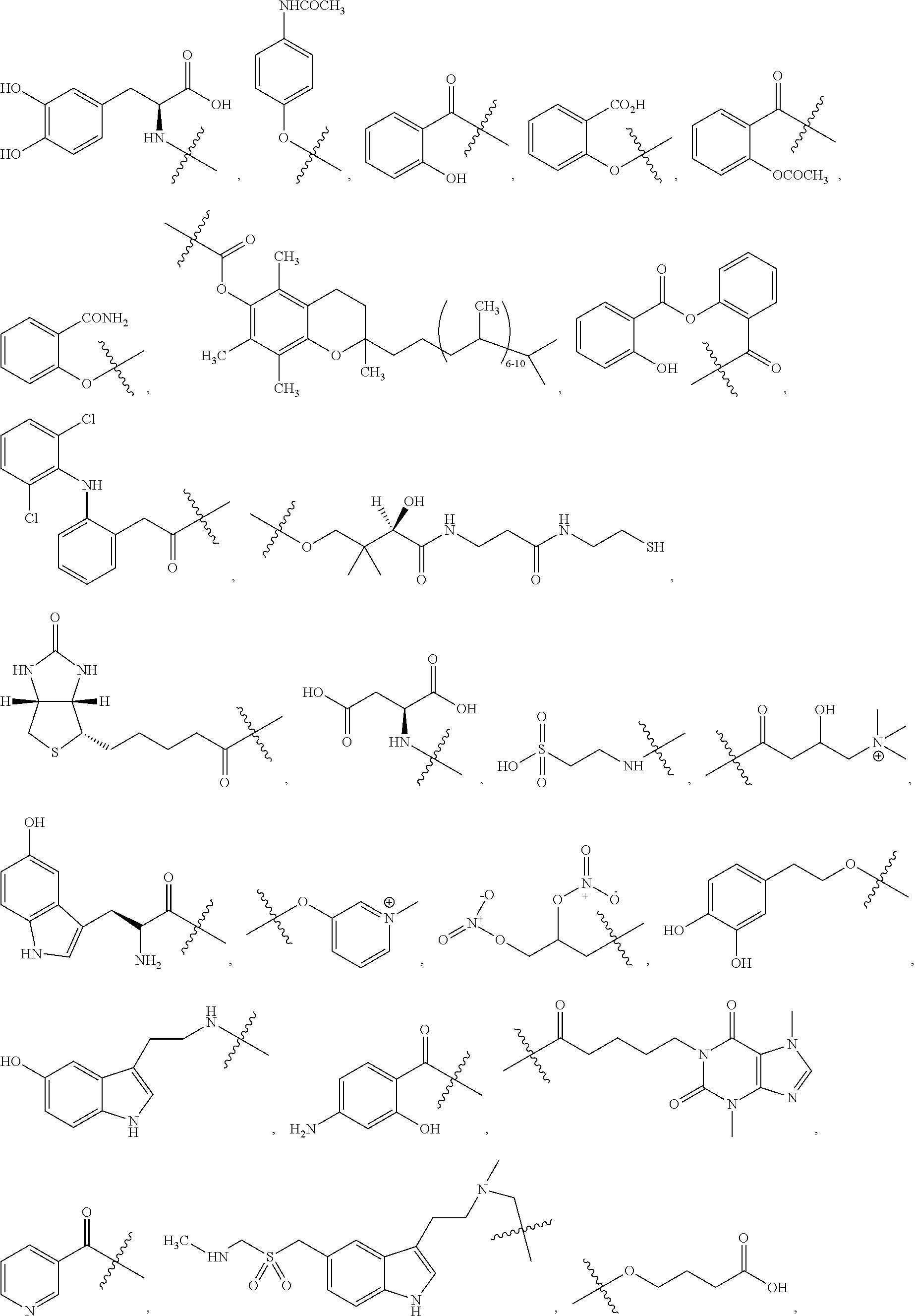

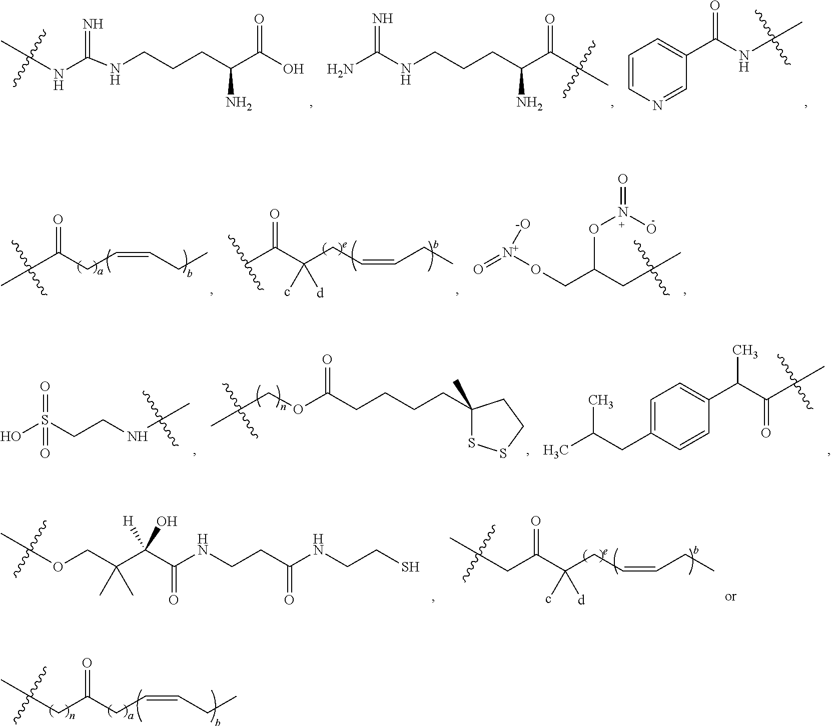

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;

Methods and compositions for the treatment of metabolic diseases. among other things, herein is provided a method of treating metabolic diseases, comprising administering to a patient in need thereof a therapeutically effective amount of compound of Formula III:

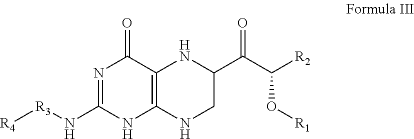

and pharmaceutically acceptable salts, hydrates, solvates, prodrugs, enantiomers, and stereoisomers thereof;

Wherein,
R¹, R² each independently represents H, D, CH₃CO, CD₃CO, CD₃, CH₃,

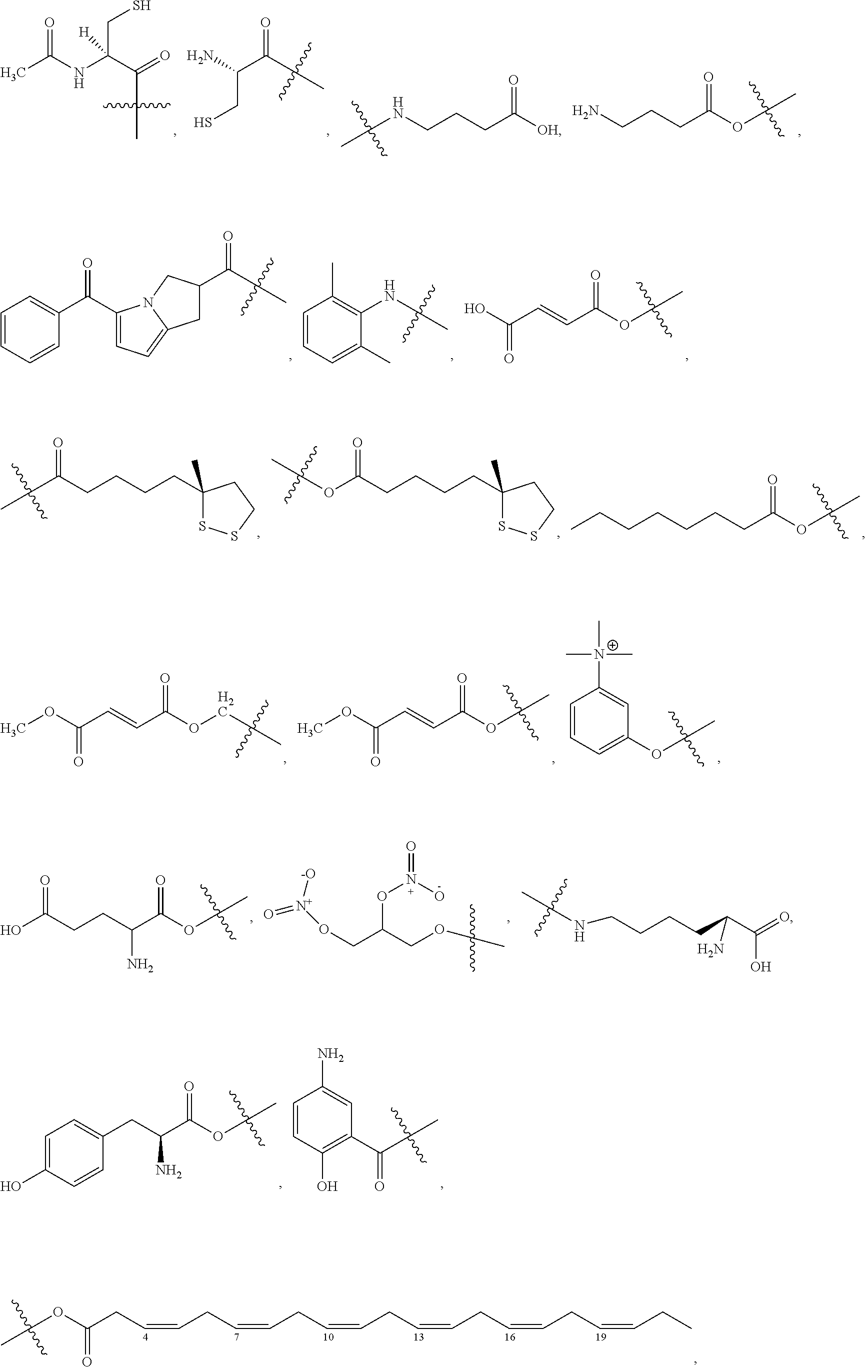

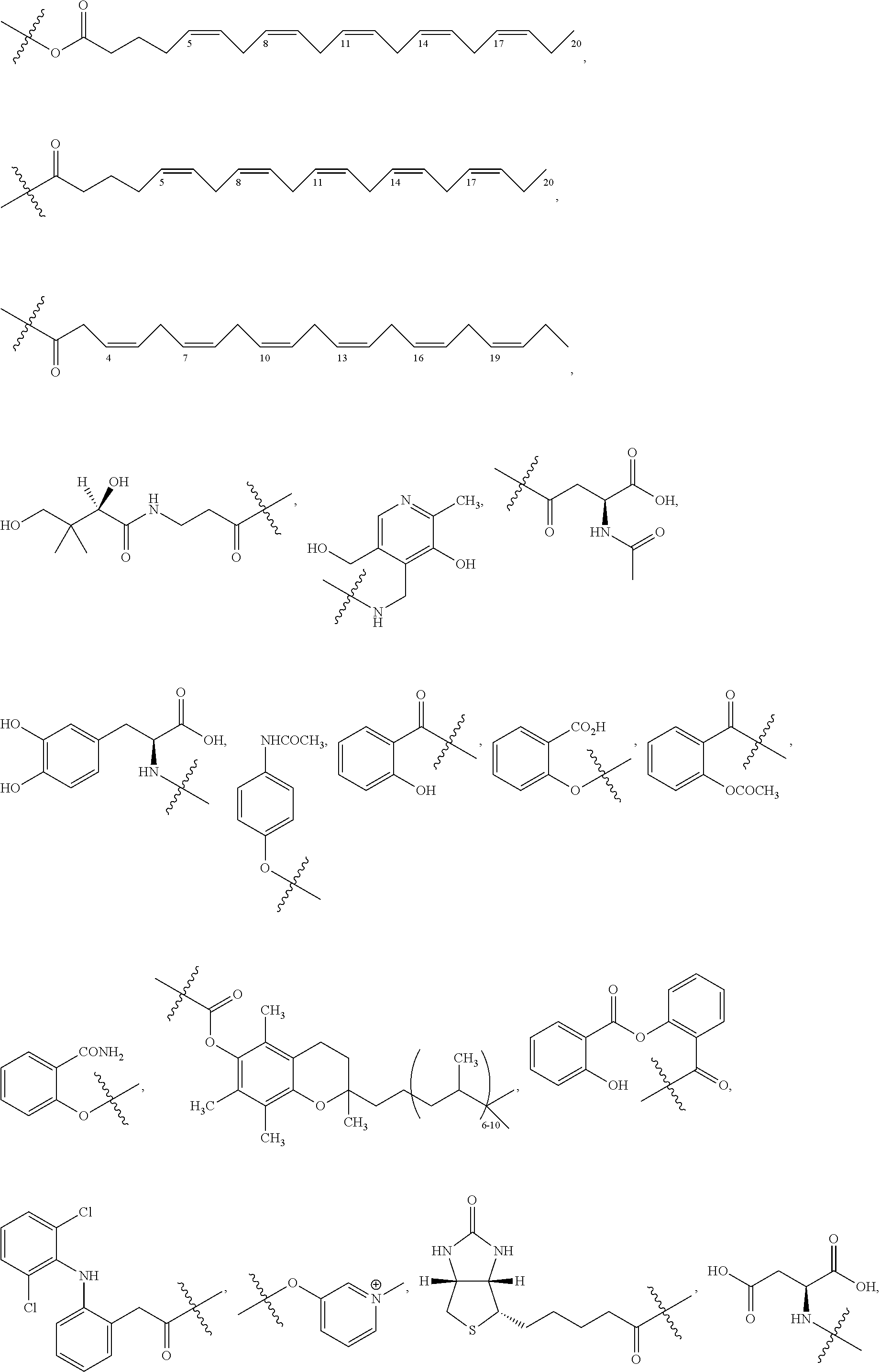

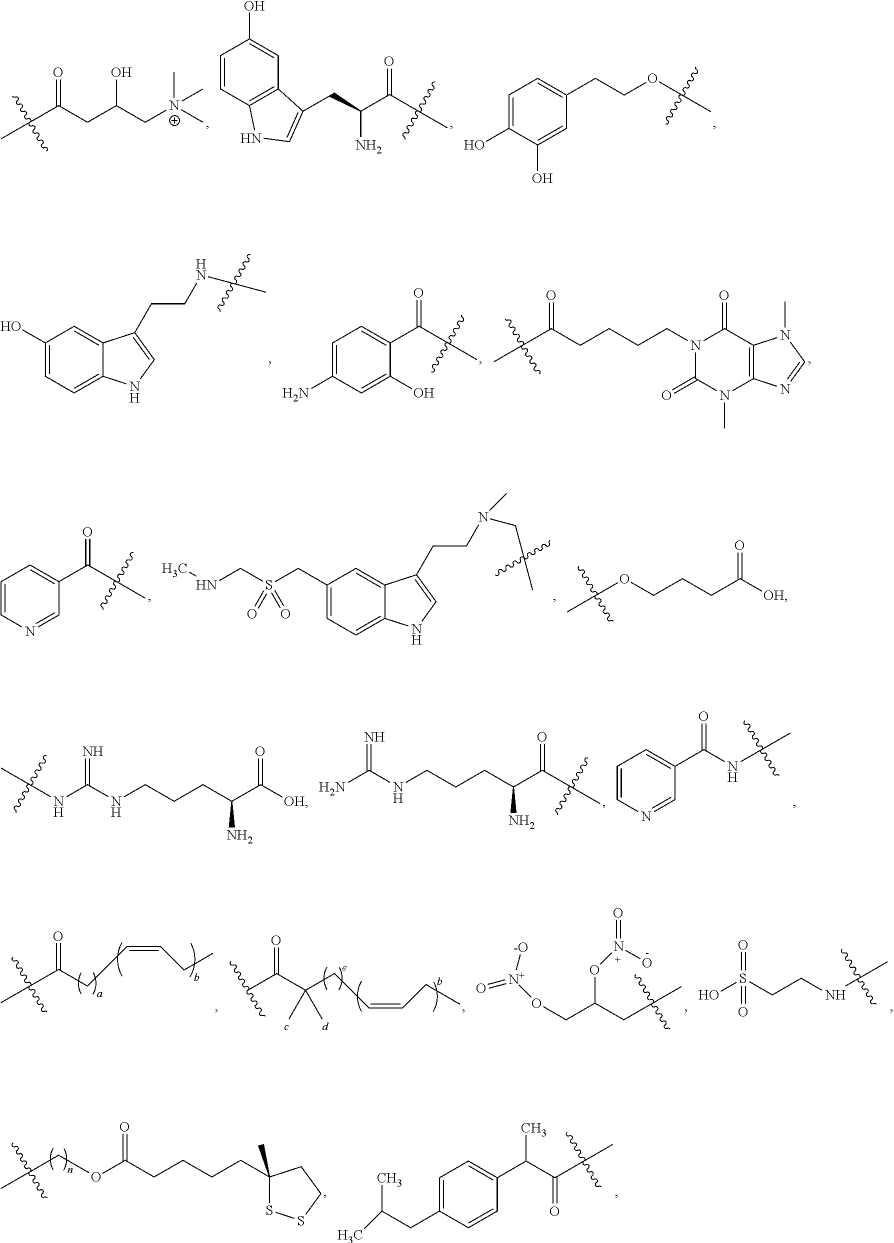

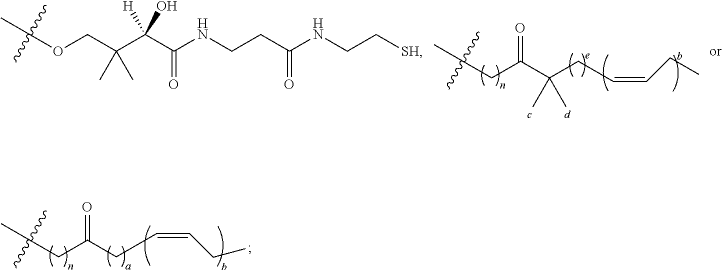

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃;
   R³ represents H, D, CH₃CO, CD₃CO, CD₃, CH₃,

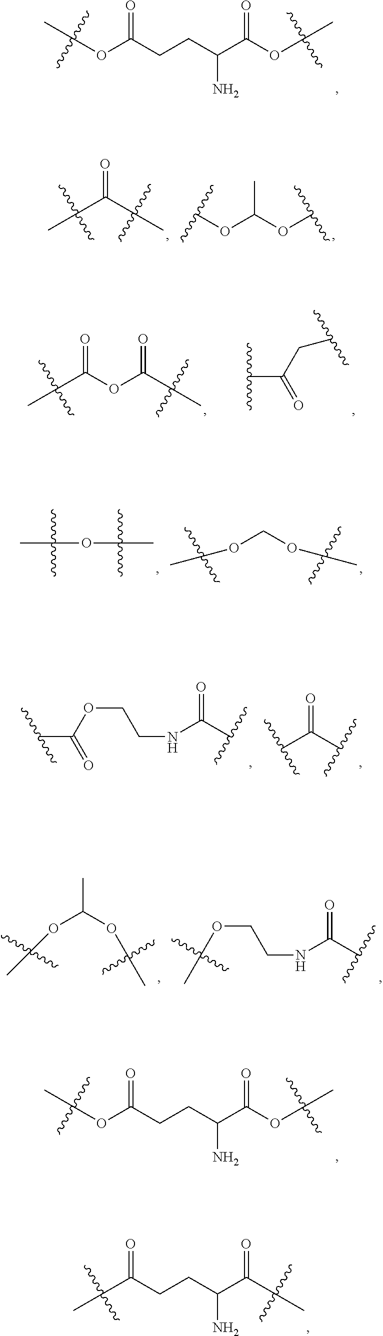

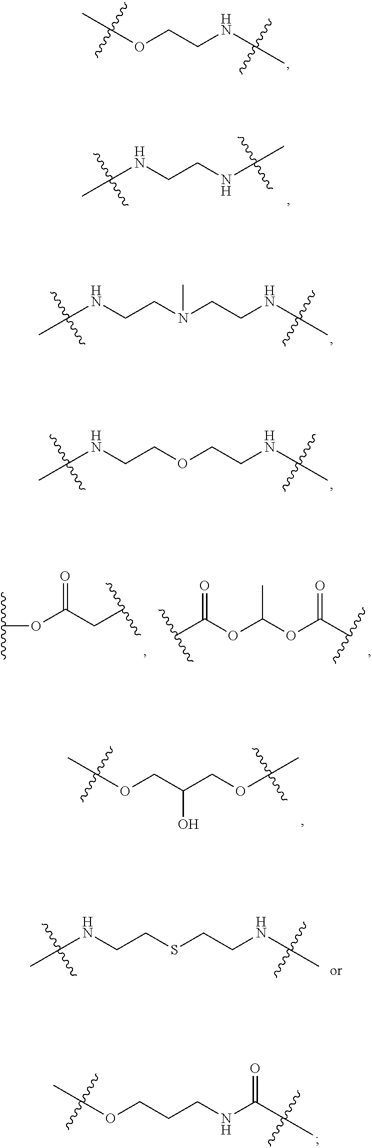

R⁴ represents D, CH₃CO, CD₃CO, CD₃,

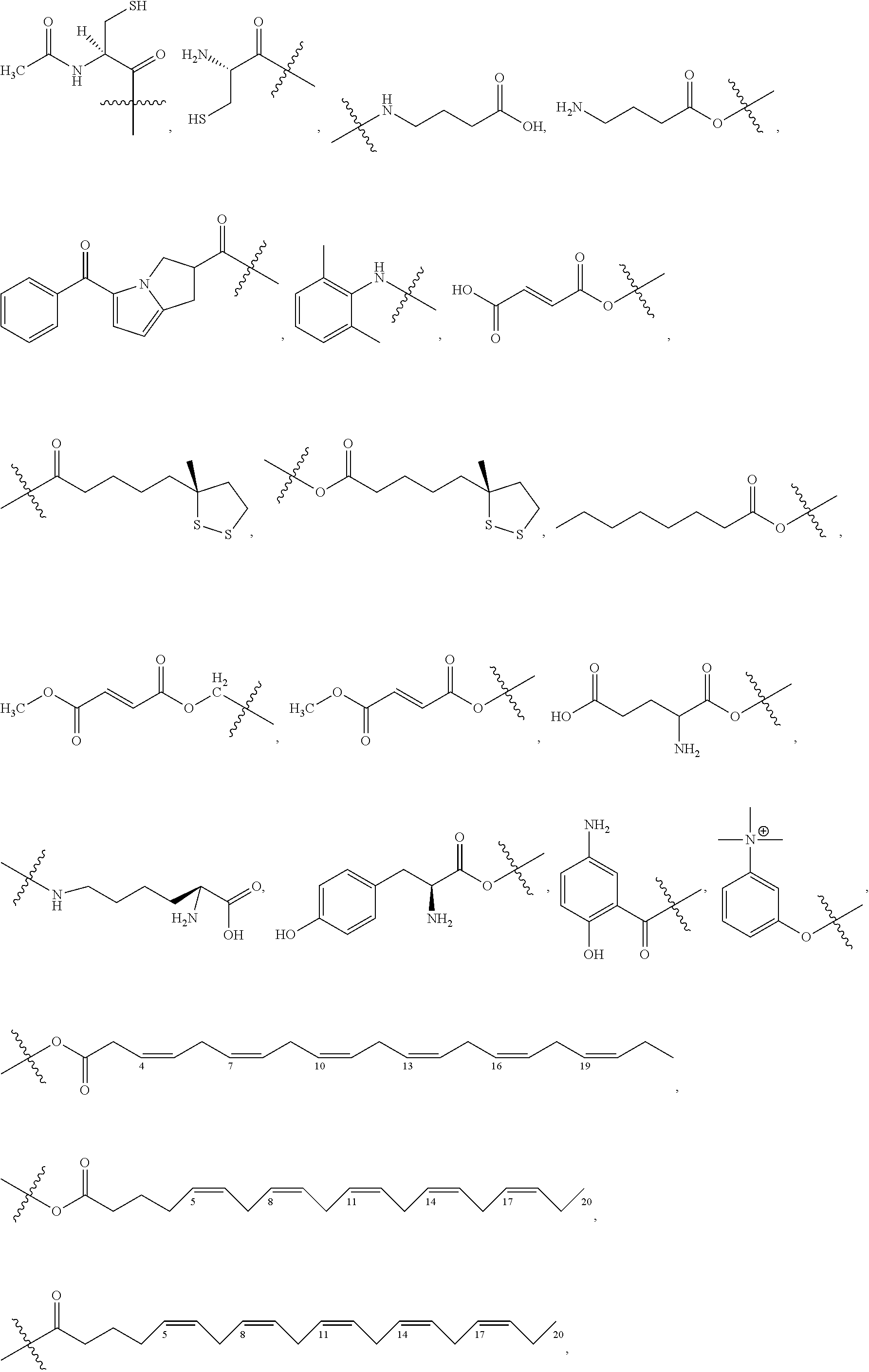

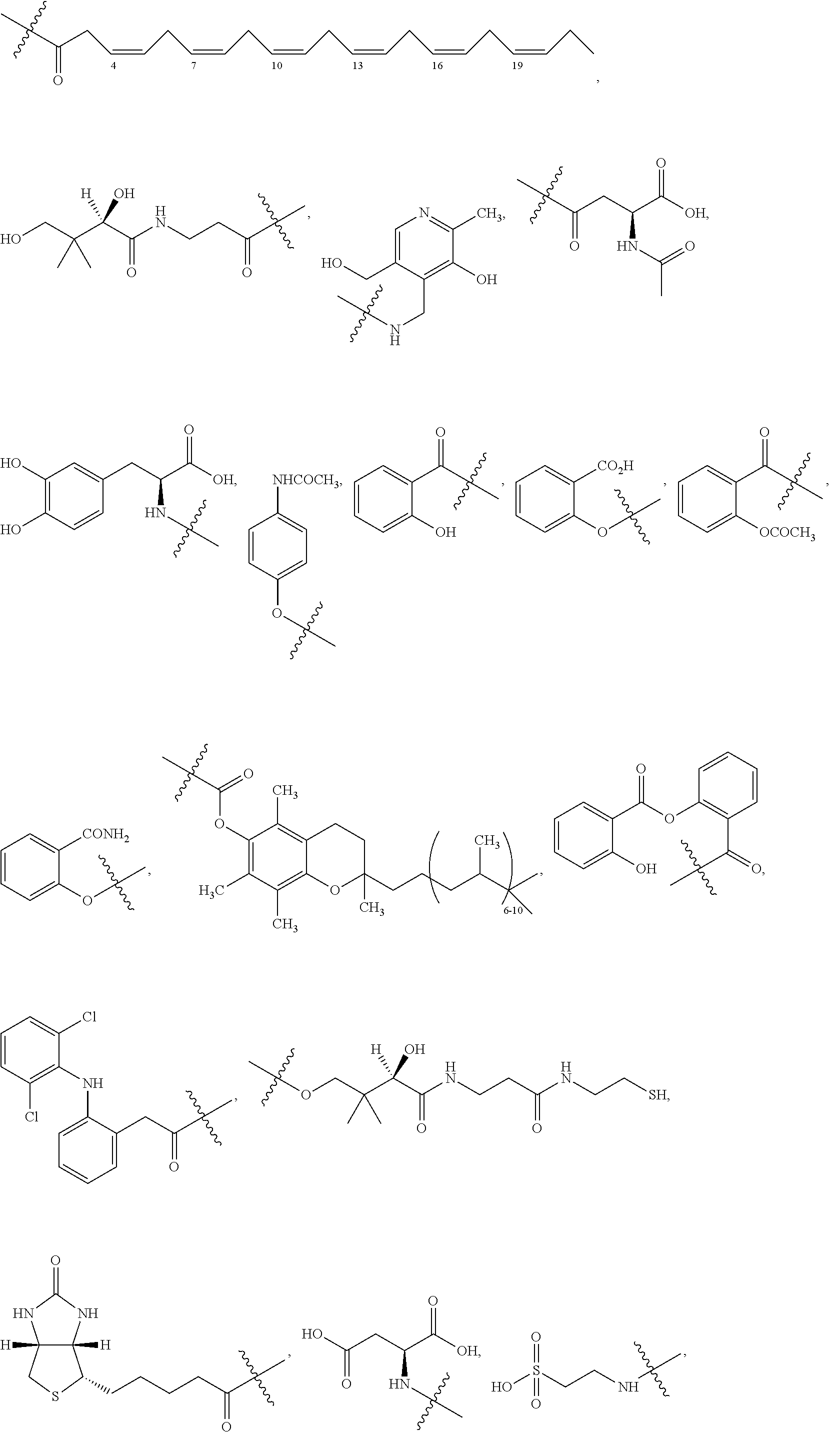

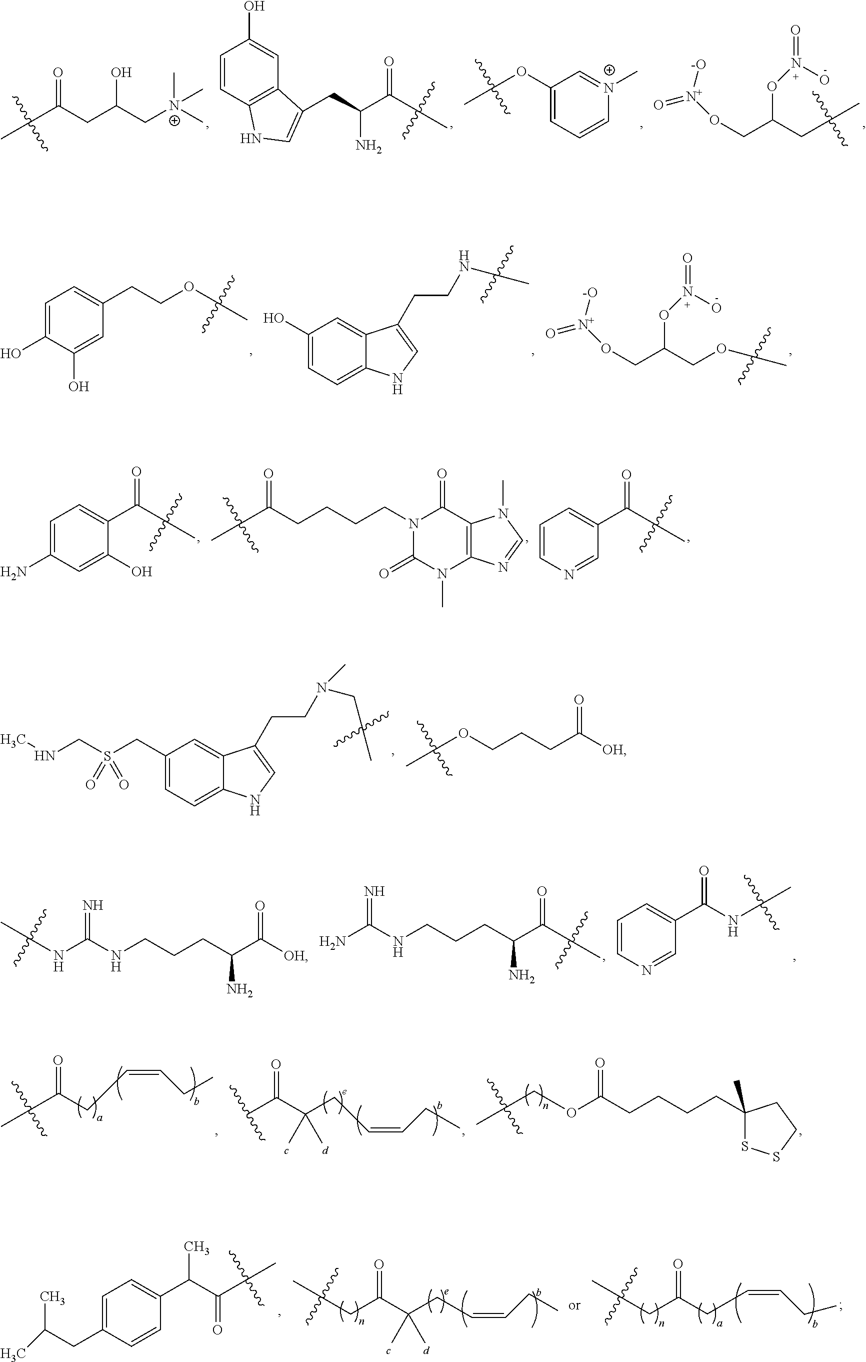

n is independently 1, 2, 3, 4 or 5;
a is independently 2, 3, or 7;
each b is independently 3, 5 or 6;
e is independently 1, 2, or 6;
c and d are each independently H, D, —OH, —OD, C₁-C₆-alkyl, —NH₂ or —COCH₃.

### METHODS OF MAKING

Example of generalized synthetic pathway useful for making compounds are depicted in scheme-I:

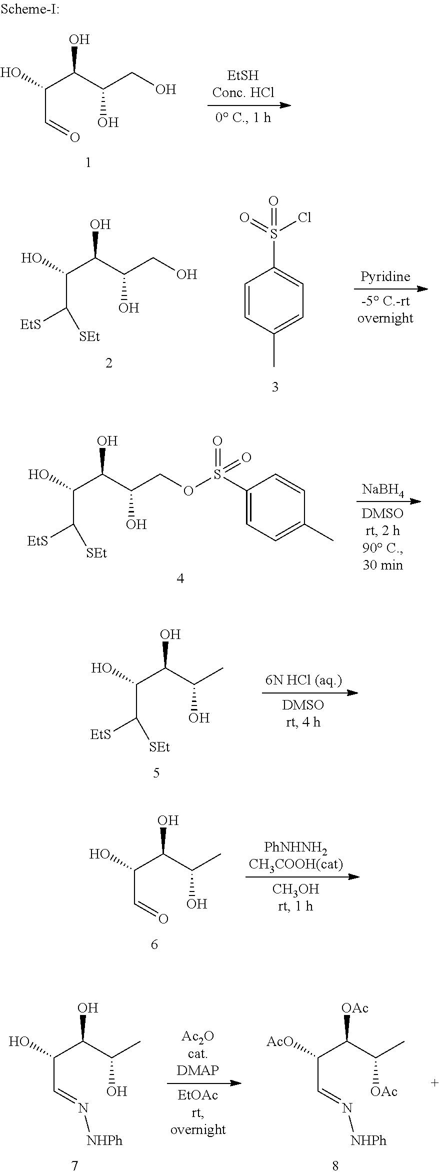

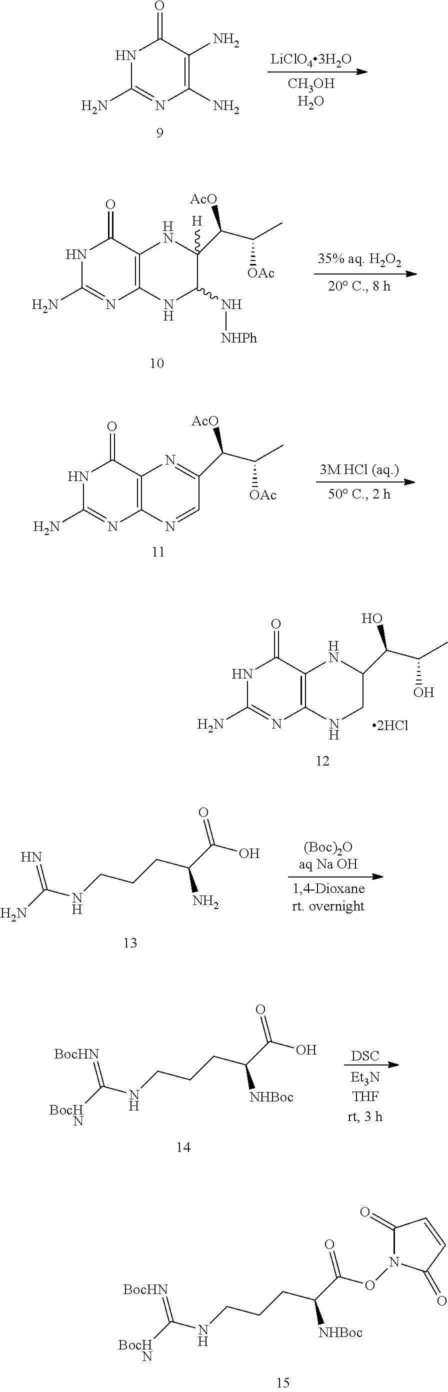

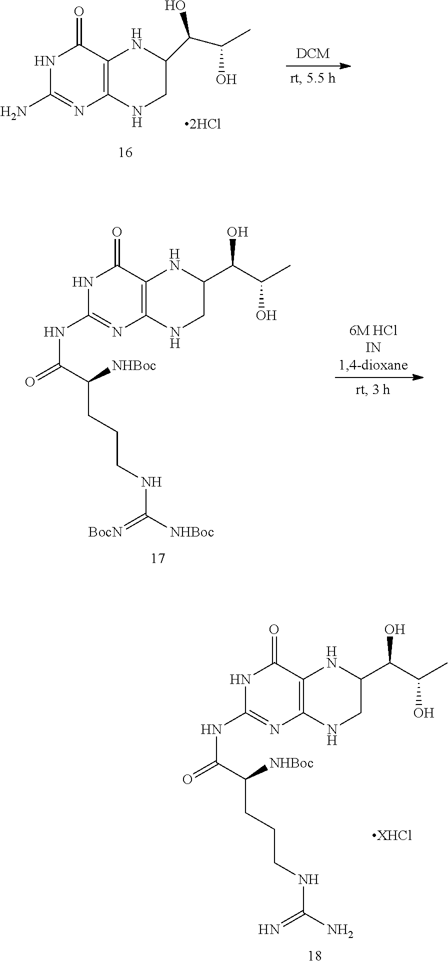

### Step-1: Synthesis of Compound 2

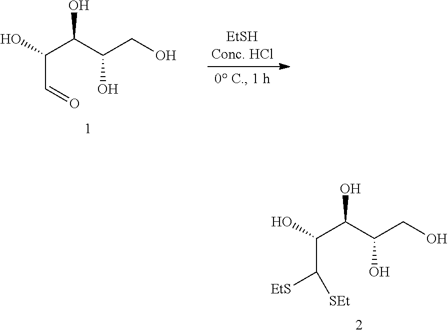

A 400 ml beaker was cooled on an ice-NaCl bath charged with 104 ml (1.4 moles) of ethylmercaptan (EtSH) and 72 ml of concentrated hydrochloric acid and the mixture was sufficiently stirred by a stirrer. After the mixture was cooled down to 0° C. of about 3 to 4 g for 10 to 20 minutes with rapid and sufficient stirring. After addition of L-(+)-arabinose 1 with total amount of 60 g (0.4 mole), stirring was continued for 1 hour, wherein the whole mixture became solid when addition was almost completed. The obtained solid was filtered, washed with 500 ml of water and dried over a Buchner funnel in air for about 2 hours. And then, the obtained solid was crystallized from 800 ml of boiling water. Water was put to boil, and when the water started to boil, the solid was added in small portions to the water with stifling in such a way that every portion was added immediately after the dissolution of the previously added portion. After all the solid was added to the water and the mixture became an almost clear solution (slightly emulsified), the clear solution was filtered in a sufficiently hot condition and the filtrate was cooled overnight. The cooled filtrate was further filtered and the obtained solid was dried in a desiccator of give 94.3 g of L-(+)-arabinose-diethyl mercaptal 2 (yield: 92%).

### Step-2: Synthesis of Compound 4

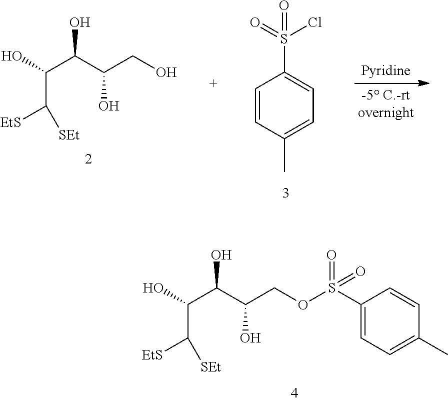

A 500 ml two necked flask equipped with a thermometer and a pressure balancing funnel was charged with 14.08 g (55 mmoles) of L-(+)-arabinose-diethylmercaptal 2. Thereto 84 ml of pyridine was added and the mixture was stirred until a clear solution was obtained. Then, the solution was sufficiently cooled to −5° C.A solution of 11.5 g (60 mmoles) of tosylchloride 3 in 40 ml of pyridine was slowly added to the above cooled solution was stifling for about 2 hours wherein the temperature of the former solution was maintained below 0 mixture was slowly heated to room temperature for 2 to 3 hours. Then, the ice-Nacl bath was removed and the flask was allowed to stand at room temperature overnight. Next morning, the reaction mixture in the flask was poured slowly into 1200 ml of ice-water with stifling and the mixture was allowed to stand for 10 minutes. The solid was filtered and washed with 30 ml of cold water 4 times. The thus obtained solid was dissolved in 200 ml of ethylacetate and the solution was washed successively with a cold dilute hydrochloric acid, dilute sodium hydrogencarbonate solution and water until the solution became neutral. After the solution was dried with sodium sulfate, the solvent was removed off. There was dissolved 19.1 g of the obtained solid residue into 75 ml of chloroform and precipitated with 300 ml of pentane. The precipitate was filtered and dried in air to give 18.4 g of the desired product 4 (yield after crystallization: 82%).

### Step-3: Synthesis of Compound 5

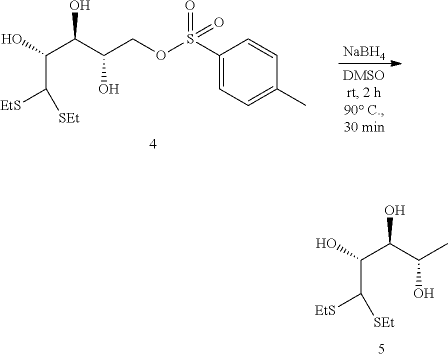

A 1000 ml round flask was charged with 4 g (105 mmoles) of NaBH₄, and thereto 100 ml of DMSO was added. The mixture was stirred by magnetic stirrer until the mixture became an almost clear solution. To the solution there was slowly added 100 ml of DMSO containing 20 g (49 mmoles) of 5-tosyl-L-(+)-arabinose-diethylmercaptal 4 for about 2 hours with stirring. After addition, the mixture was further stirred for half an hour at room temperature. Then, the mixture was heated on a water bath for one hour at 90 dryness on a water bath of 90 mixture was cooled and the obtained residue was decomposed with ice water. A small amount of scum was filtered off and the obtained clear filtrate was made slightly acidic with 5% acetic acid. After the filtrate was thoroughly extracted with ether, the ether layer was dried with sodium sulfate and the solvent was evaporated from the ether layer to give 9.5 g of 5-deoxy-L-(+)-arabinose-diethylmercaptal 5 as a white solid residue (yield: 81%).

### Step-4: Synthesis of Compound 6

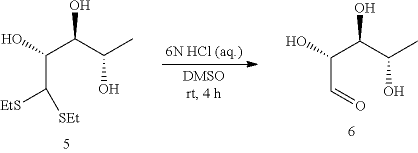

9.5 g (40 mmoles) of 5-deoxy-L-(+)-arabinose-diethylmercaptal obtained in the above reaction was kept in 11 g (140 mmoles) of DMSO in 100 ml of 6N hydrochloric acid at room temperature and the mixture was mechanically stirred for about 4 hours until all solid was dissolved into a solution. After further stirring for 1 hour, a mixture comprising two homogeneous liquids was obtained. The mixture was transferred to a separatory funnel and allowed to precipitate. The lower aqueous layer was separated, cooled on an ice-NaCl bath and adjusted to pH 6.5 with 6N sodium hydroxide. The solvent was evaporated under condition close to vacuum as far as possible and the precipitated sodium chloride was filtered off through a glass filter crucible. The precipitate was thoroughly washed with 10 ml of ethanol 5 times. The collected filtrate was evaporated and once more sodium chloride was filtered. The procedures of the above filtration, washing with alcohol (provided that methanol which dissolves 5-deoxy-L-arabinose more sufficiently was employed on and after the second washing) and evaporation, which were repeated 5 times so that the separation of sodium chloride was unnecessary, gave 5.0 g of 5-deoxy-L-arabinose 6 as a syrup (yield: 93%).

### Step-5: Synthesis of Compound 7

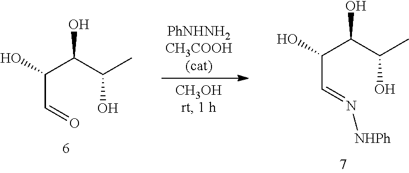

There was added 5.0 g (37.3 mmoles) of 5-deoxy-L-(+)-arabinose 6 obtained above reaction to 80 ml of methanol and the mixture was stirred to give a clear solution. With stifling there was added dropwise 4.5 g (42 mmoles) of phenylhydrazine to the obtained mixture and thereto further a drop of glacial acetic acid was added. Thus obtained yellow solution was allowed to stand for 1 hour at room temperature and then the solution was evaporated under a condition close to vacuum as far as possible to give a thick viscous residue. After the residue was washed with 30 ml of ether twice, the residue was dissolved in 150 ml of ethyl acetate. The thus obtained solution was washed with about 40 ml of water twice, the organic layer was dried with sodium sulfate and the solvent was evaporated under vacuum. There remained a pale yellow skin-like solid on the bottom of the flask. The solid was washed with 30 ml of ether twice (for half an hour per each washing) and dried to give 8.0 g of 5-deoxy-L-arabinose-phenylhydrazone 7 (yield: 96%).

### Step-6: Synthesis of Compound 8

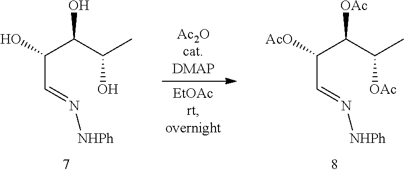

To the ethyl acetate (100 ml) solution of 5-deoxy-L-arabinose phenylhydrazone obtained in the previous step, 9.0 g (0.074 mol) of 4-dimethylaminopyridine (DMAP) was added and dissolved therein. Then 120.82 g (1.183 mol) of acetic anhydride was added by dripping to the solution at an ambient temperature of 10° C. After stifling overnight at the same ambient temperature, 250 ml of water was added to the solution, which was then stirred for 30 minutes. After allowing the solution to stand, it was separated into a water layer and an organic layer, and a 20% aqueous solution of sodium hydroxide was added to the organic layer up to its neutralization. Then after allowing the solution to stand, its organic layer was separated out and dried over anhydrous sodium sulfate. When the thus treated ethyl acetate solution was subjected to vacuum concentration, an ethyl acetate solution of triacetoxy-5-deoxy-L-arabinose phenylhydrazone 8 was obtained.

### Step-7: Synthesis of Compound 10

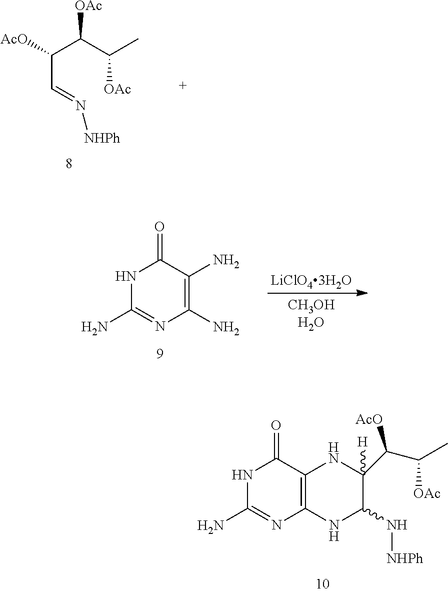

To the ethyl acetate solution of triacetoxy-5-deoxy-L-arabinose phenylhydrazone obtained in the previous step, were added 500 ml of methanol, 41.74 g (0.296 mol) of 6-4iydroxy-2,4,5-triaminopyrimidine 9 and 300 ml of water in the cited order. Further, 23.73 g (0.140 mol) of lithium perchlorate trihydrate dissolved in 200 ml water was added thereto and the resultant solution was stirred at 50° C. for 6 hours to obtain an aqueous solution of compound 10 which was extracted with DCM and evaporation of DCM layer and purification through column yields the intermediate 10.

### Step-8: Synthesis of Compound 11

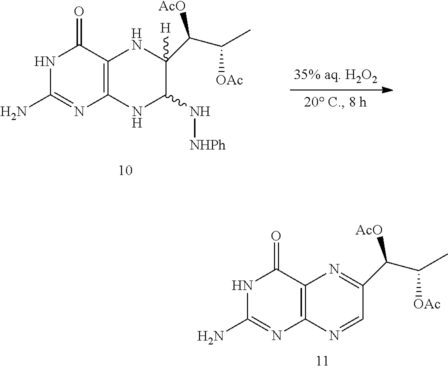

A 35% hydrogen peroxide solution (1.405 mol) was added by dripping to the aqueous solution of compound 10 obtained in the preceding example and the resultant mixture was stirred at 20° C. for 8 hours. Crystals deposited there were separated out by filtration and rinsed with water and methanol to yield the intermediate 11.

### Step-9: Synthesis of Compound 12

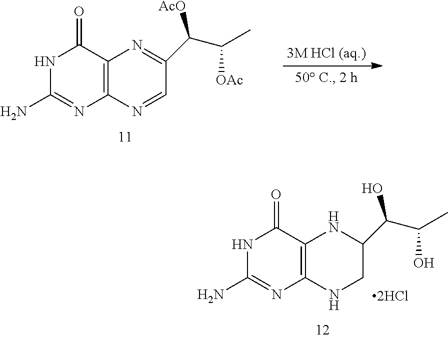 [SMILES: CC(=O)O[C@@H](C)[C@H](OC(C)=O)c1cnc2nc(N)[nH]c(=O)c2n1 ; C[C@H](O)[C@H](O)C1CNc2nc(N)[nH]c(=O)c2N1]

Compound 11 obtained in the preceding example was suspended in 3M hydrochloric acid and the resultant suspension was stirred at 50° C. for 2 hours. After decoloring with activated charcoal, the reaction solution was neutralized with 28% ammonia water. Subsequently, crystals deposited there were separated out by filtration and dried to obtain 23.13 g of compound 12.

### Step-10: Synthesis of Compound 14

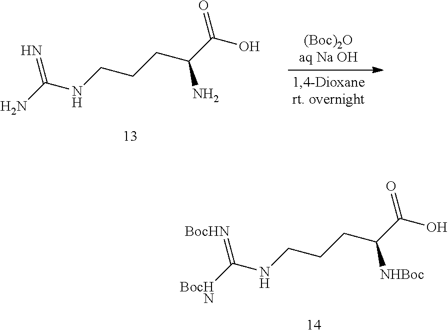

Compound 13 by dissolving (0.17 mol) L-arginine and (0.288 mol) sodium hydroxide in 300 ml water. Di-t-butyl dicarbonate (42 g; 0.68 mol) was added to the solution, but failed to dissolve, giving a nonhomogeneous mixture. Tetrahydrofuran (300 ml) and methanol (150 ml) were added to the mixture, the solids dissolved and a homogeneous solution was obtained. The solution was stirred for 5.5 hours while monitoring pH, with 5.5 g NaOH added to the solution after the pH dropped to about 7.0. The organic solvents were removed from the reaction mixture with a rotary evaporator, and the remaining aqueous solution was poured into 1000 ml water and acidified with aq.citric acid to a pH of between 3 and 4 with mechanical stirring. The white solid, which precipitated, was recovered by filtration and dried in an oven under high vacuum to yield the intermediate 14.

### Step-11: Synthesis of Compound 15

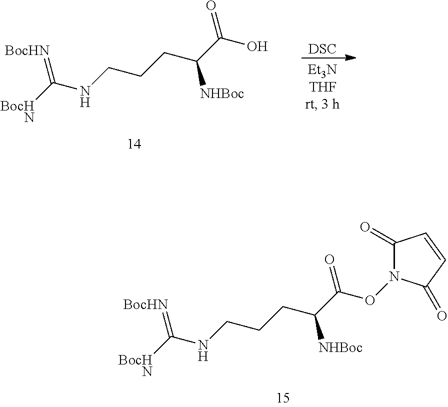

Compound 14 (40 mmol) and N,N′-disuccinimidyl carbonate (11.26 g; 44 mmol) were combined with 14 ml triethylamine in 200 ml tetrahydrofuran (THF). The reaction mixture was stirred under a nitrogen atmosphere at room temperature for three hours. Thin layer chromatography (TLC) was then performed using a solvent mixture of 50% hexane and 50% ethyl acetate to confirm that the reaction had reached completion. The reaction mixture was next added to 100 ml of a saturated aqueous NaHCO₃ solution, and extracted three times with 250 ml ethyl acetate. The organic extracts were combined, and dried over anhydrous sodium sulfate. The solvent was removed in a rotary evaporator, and the solid residue was dissolved in a mixture of 40 ml ethyl acetate and 60 ml hexane. The solution was allowed to stand overnight at room temperature, and a solid material recrystallized from the solution. This product, identified as activated ester 15 was isolated with a yield of (77%).

### Step-12: Synthesis of Compound 17

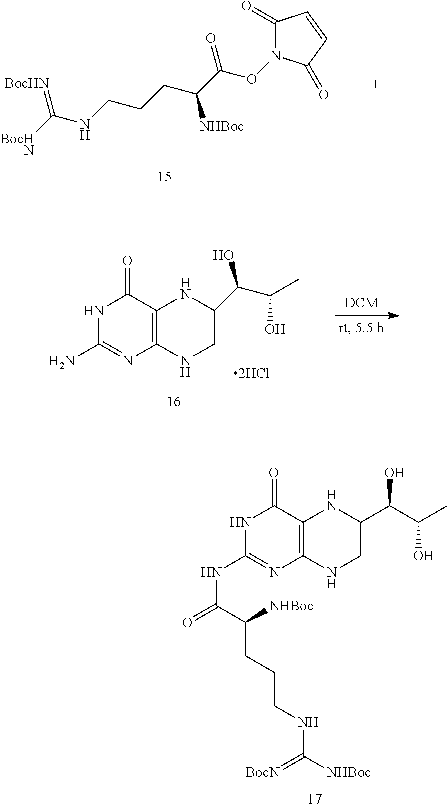

Activated ester 15 (3.22 mmol), was dissolved in 16 ml dichloromethane (DCM). The solution was then added drop-wise to a room temperature solution of compound 16 (3.22 mmol) in DCM under a nitrogen atmosphere. The reaction mixture was stirred for 5.5 hours. Dichloromethane (100 ml) was then added to the reaction mixture, and the resulting solution was extracted with a saturated aqueous solution of sodium bicarbonate (20 ml). The organic phase was separated from the aqueous phase and dried over sodium sulfate. The solvent was then evaporated, leaving a solid residue identified as intermediate 17.

### Step-13: Synthesis of Compound 18

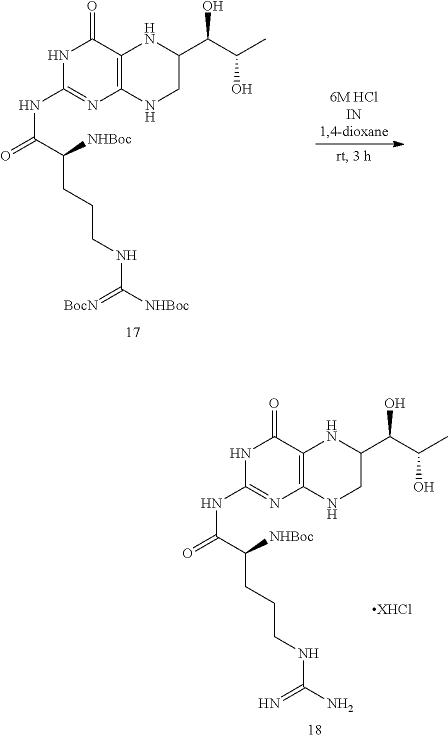

To the compound 17 was added 6M HCl and stirred the reaction mixture at room temperature for 3 h. After completion of the reaction the solvent was evaporated in rotator evaporator to the hydrochloride salt of final compound 18. Chemical Formula: C20H35N9O6; Molecular Weight: 497.55; Elemental Analysis: C, 48.28; H, 7.09; N, 25.34; O, 19.29.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

### EQUIVALENTS

The present disclosure provides among other things compositions and methods for treating metabolic diseases and their complications. While specific embodiments of the subject disclosure have been discussed, the above specification is illustrative and not restrictive. Many variations of the systems and methods herein will become apparent to those skilled in the art upon review of this specification. The full scope of the claimed systems and methods should be determined by reference to the claims, along with their full scope of equivalents, and the specification, along with such variations.

### INCORPORATION BY REFERENCE

All publications and patents mentioned herein, including those items listed above, are hereby incorporated by reference in their entirety as if each individual publication or patent was specifically and individually indicated to be incorporated by reference. In case of conflict, the present application, including any definitions herein, will control.

1. A compound of Formula I: 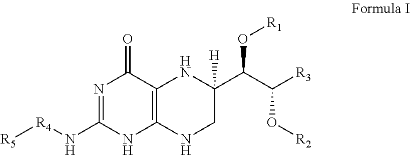
and pharmaceutically acceptable salts, hydrates, solvates, prodrugs, enantiomers, and stereoisomers thereof;
Wherein,
R¹, R², R³ each independently represents H, D, CH₃CO, CD₃CO, CD₃, CH₃, 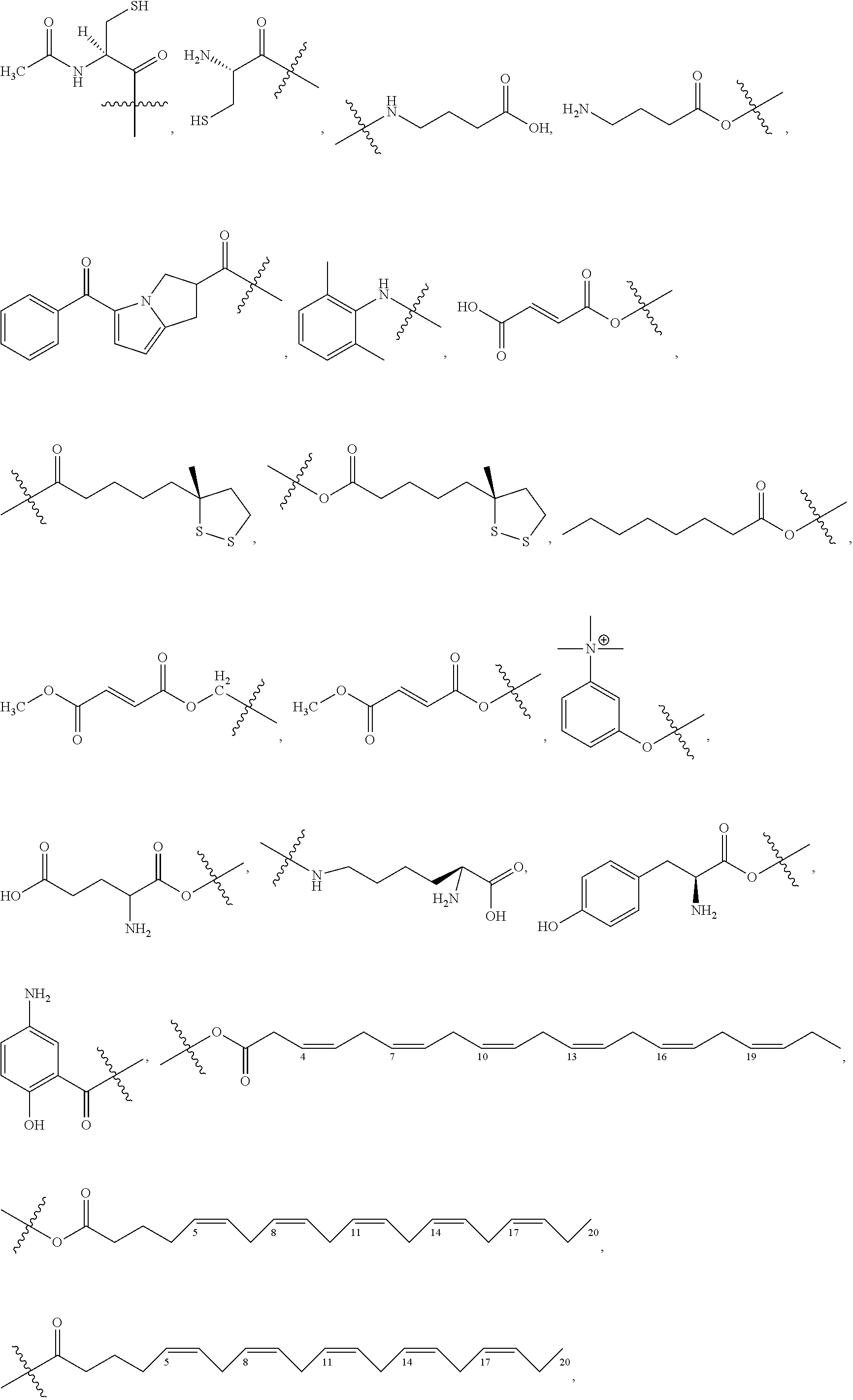 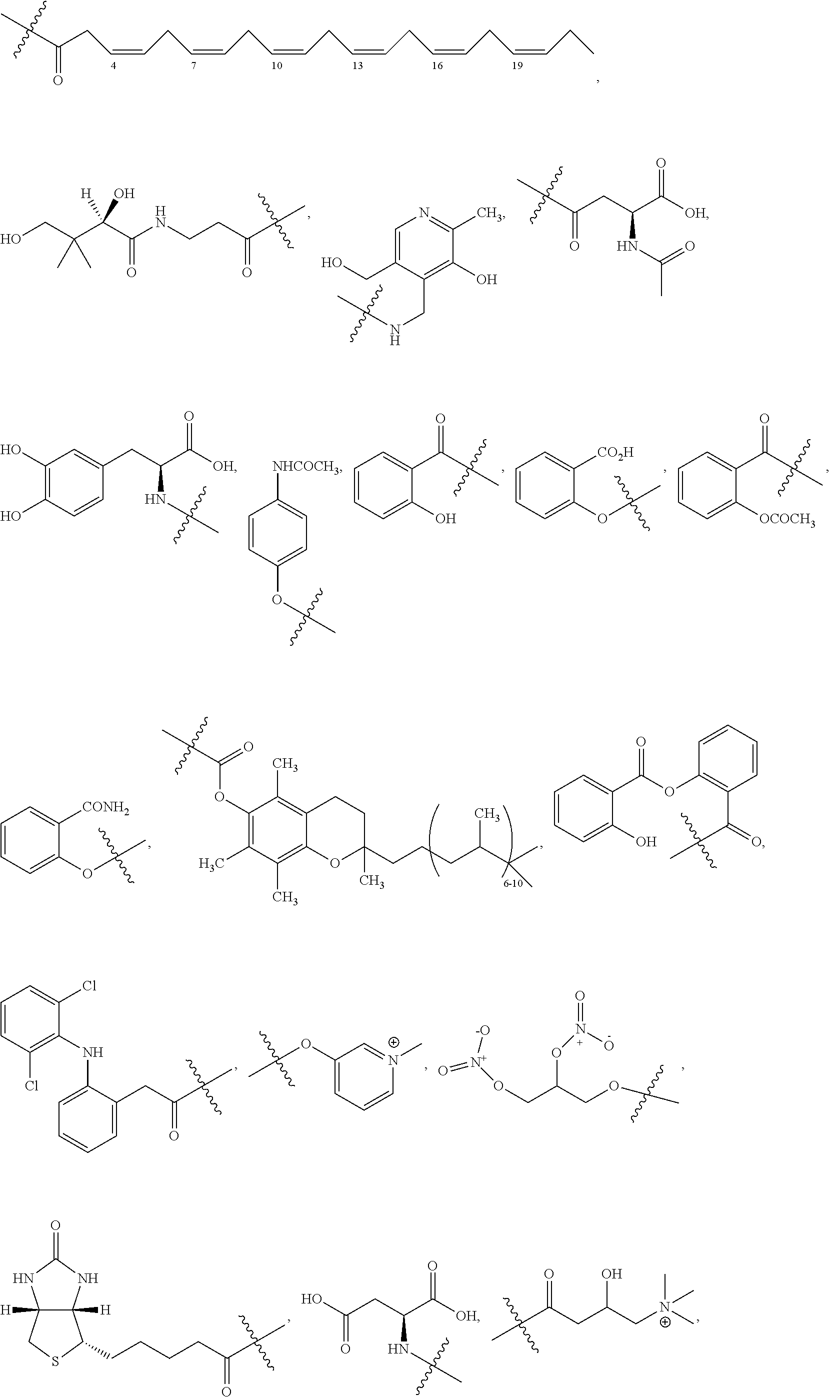 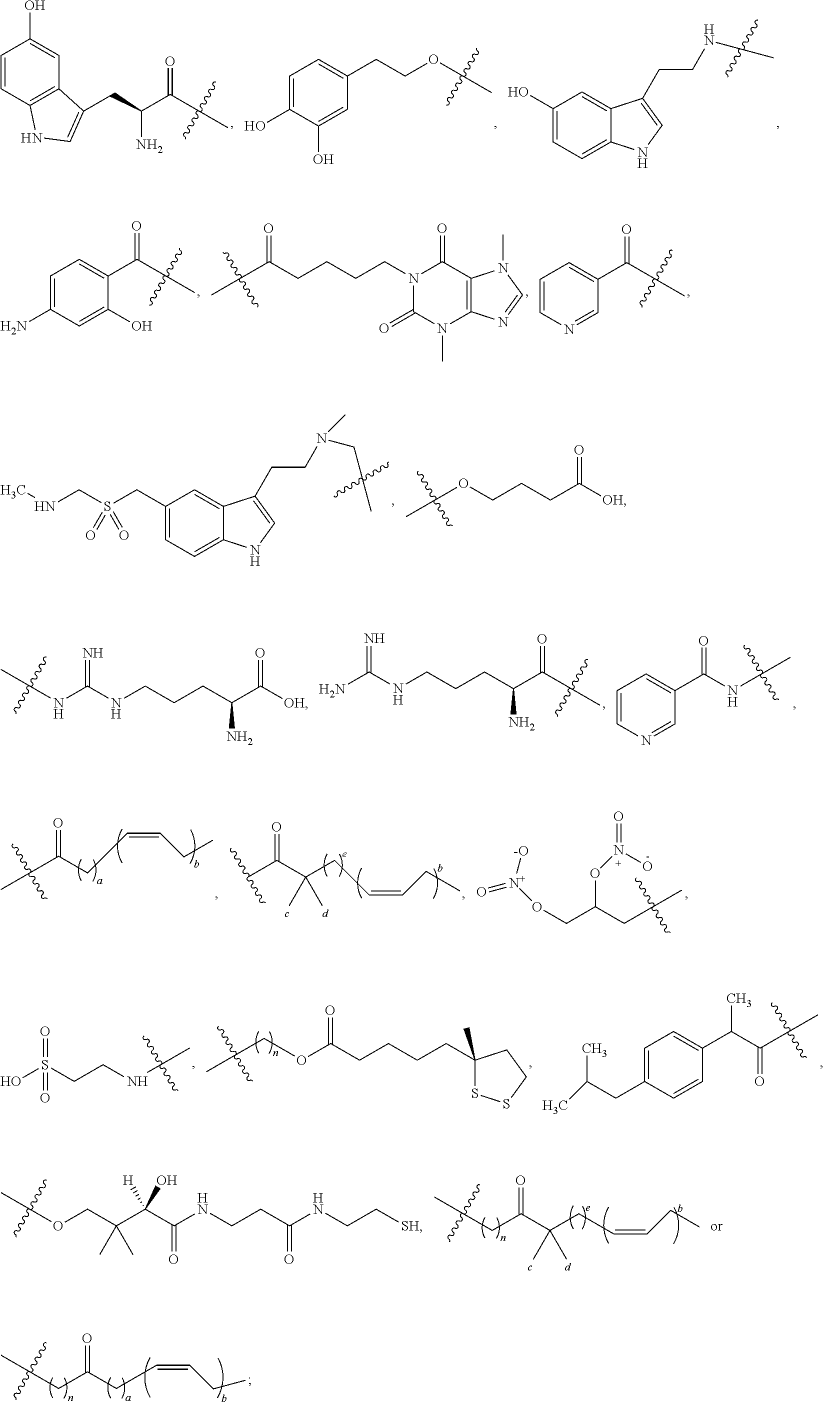
n is independently 1, 2, 3, 4 or 5;
a is independently 2, 3 or 7;
each b is independently 3, 5 or 6;
e is independently 1, 2 or 6;
c and d are each independently H, D, —OH, —OD, C₁-C₆-alkyl, —NH₂ or —COCH₃;
R⁴ represents H, D, CH₃CO, CD₃CO, CD₃, CH₃, 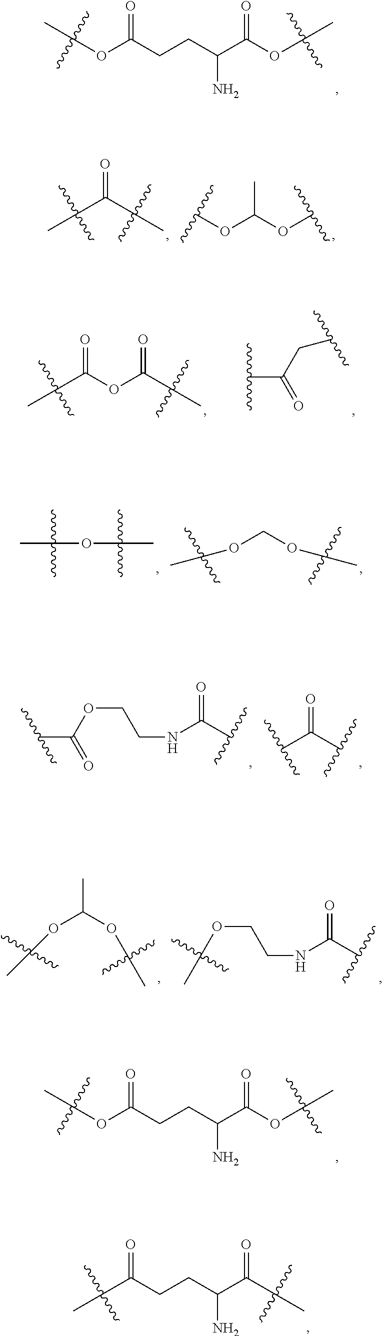 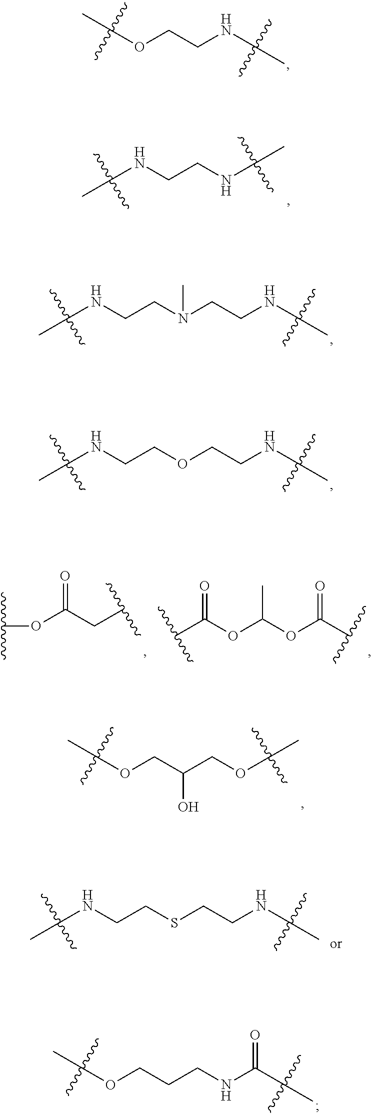
R⁵ represents D, CH₃CO, CD₃CO, CD₃, 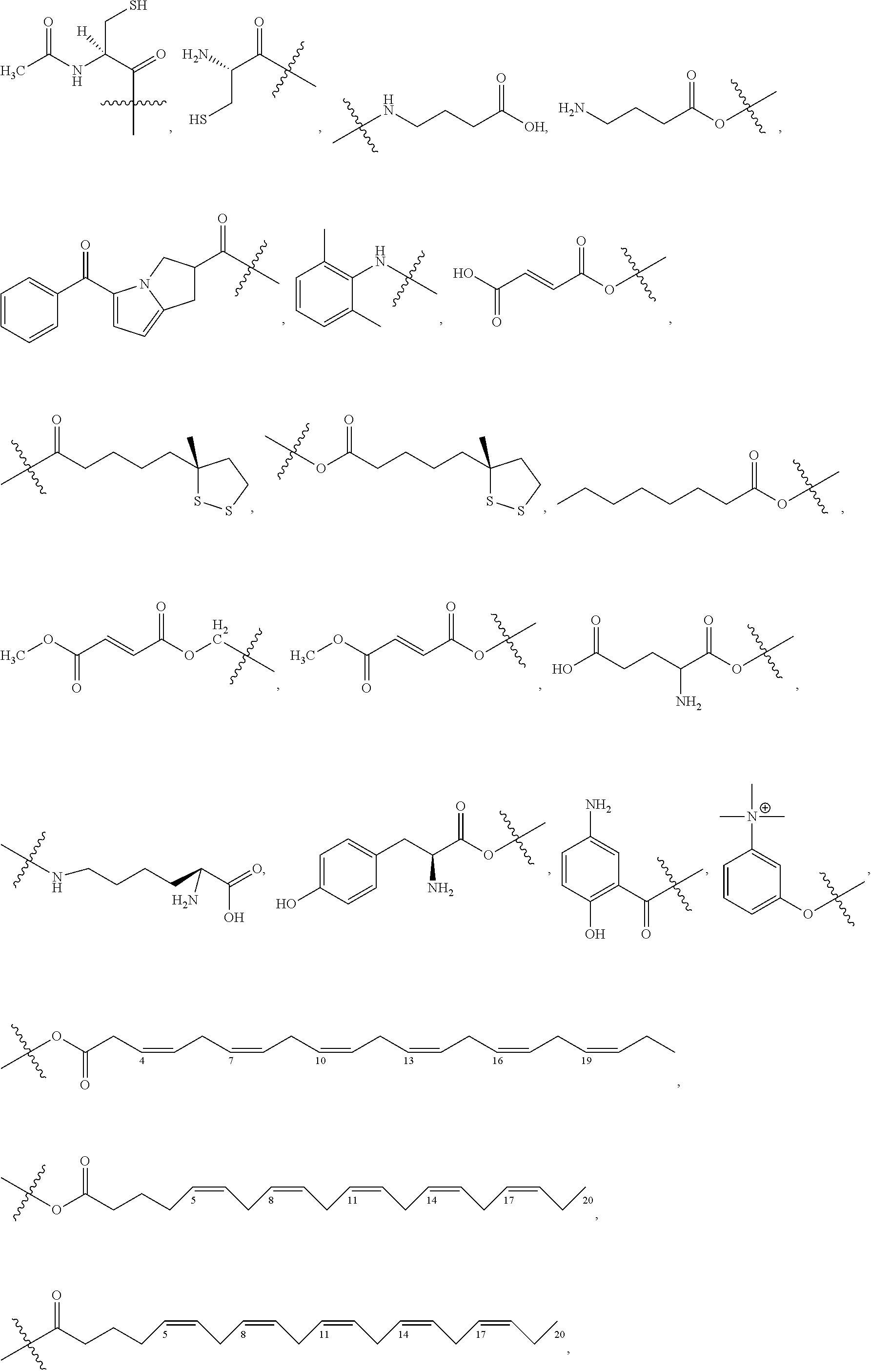 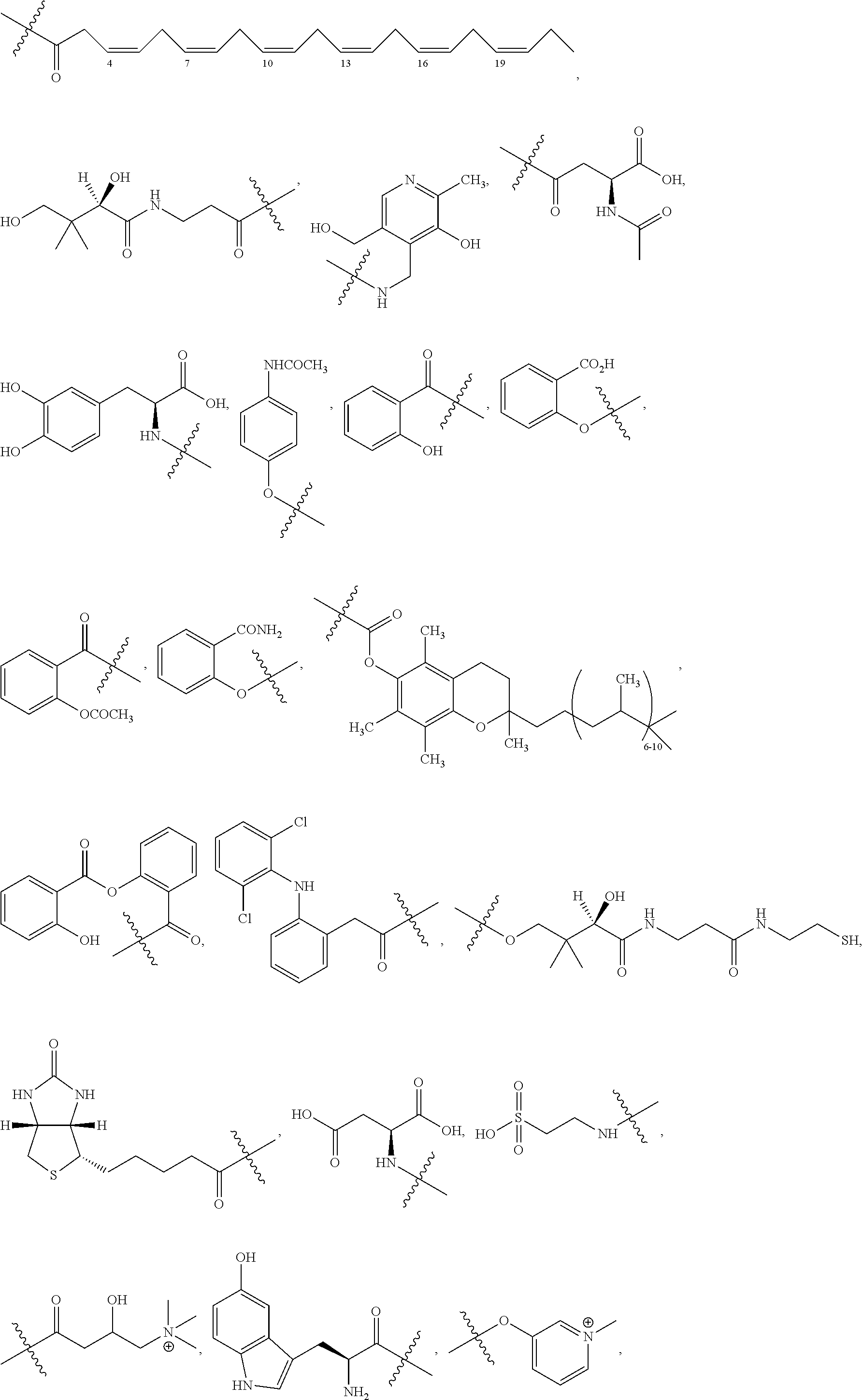 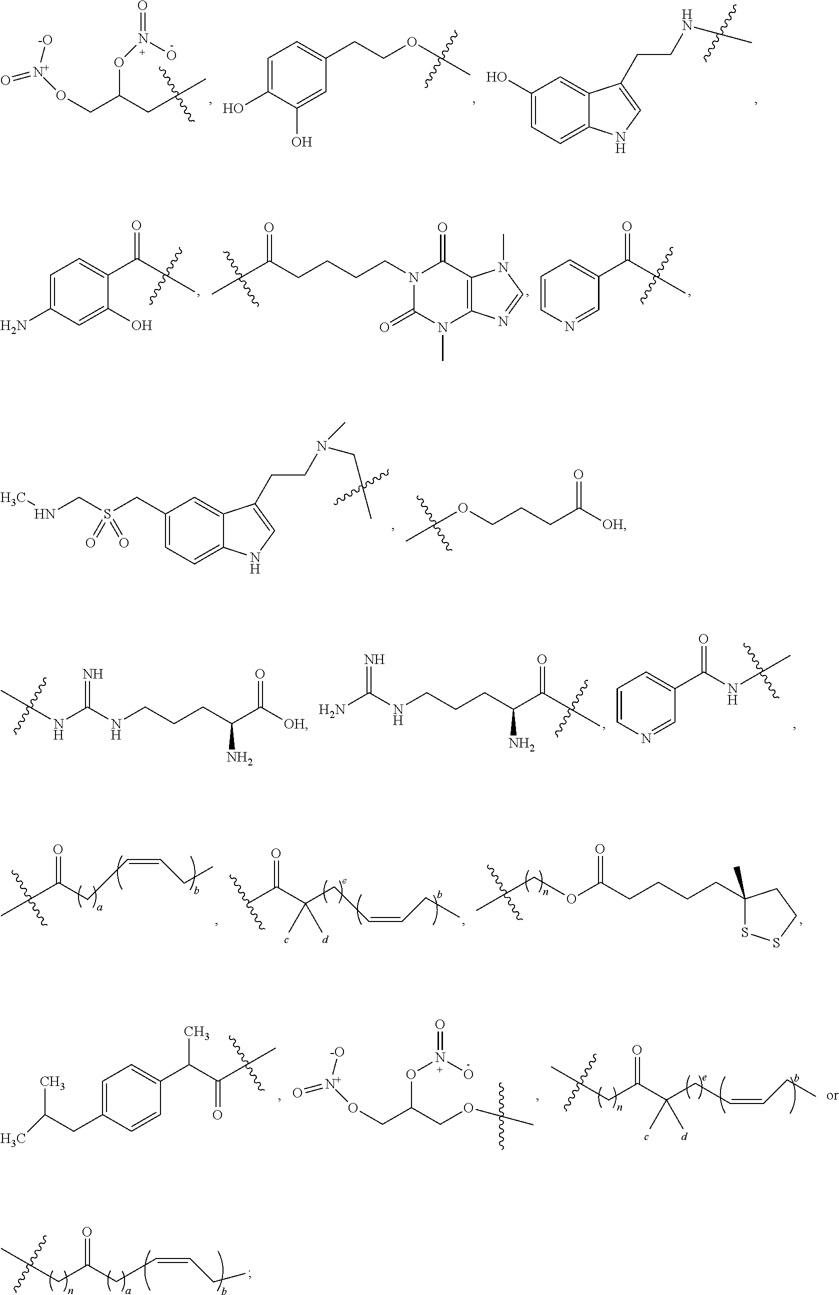
n is independently 1, 2, 3, 4 or 5;
a is independently 2, 3 or 7;
each b is independently 3, 5 or 6;
e is independently 1, 2 or 6;
c and d are each independently H, D, —OH, —OD, C₁-C₆-alkyl, —NH₂ or —COCH₃;

2. A compound of Formula II: 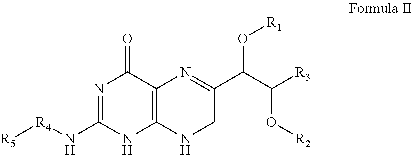
and pharmaceutically acceptable salts, hydrates, solvates, prodrugs, enantiomers, and stereoisomers thereof;
Wherein,
R¹, R², R³ each independently represents H, D, CH₃CO, CD₃CO, CD₃, CH₃, 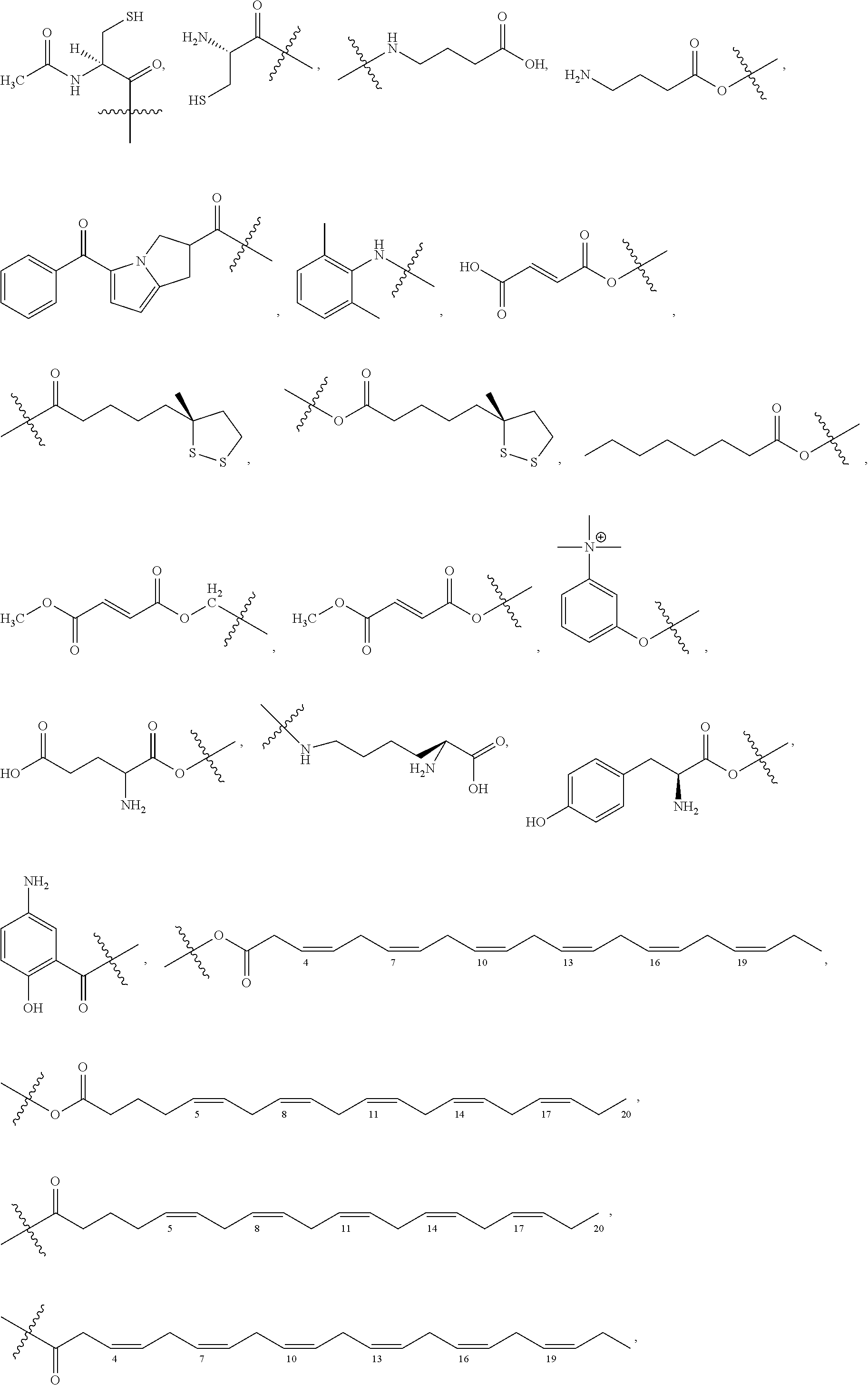 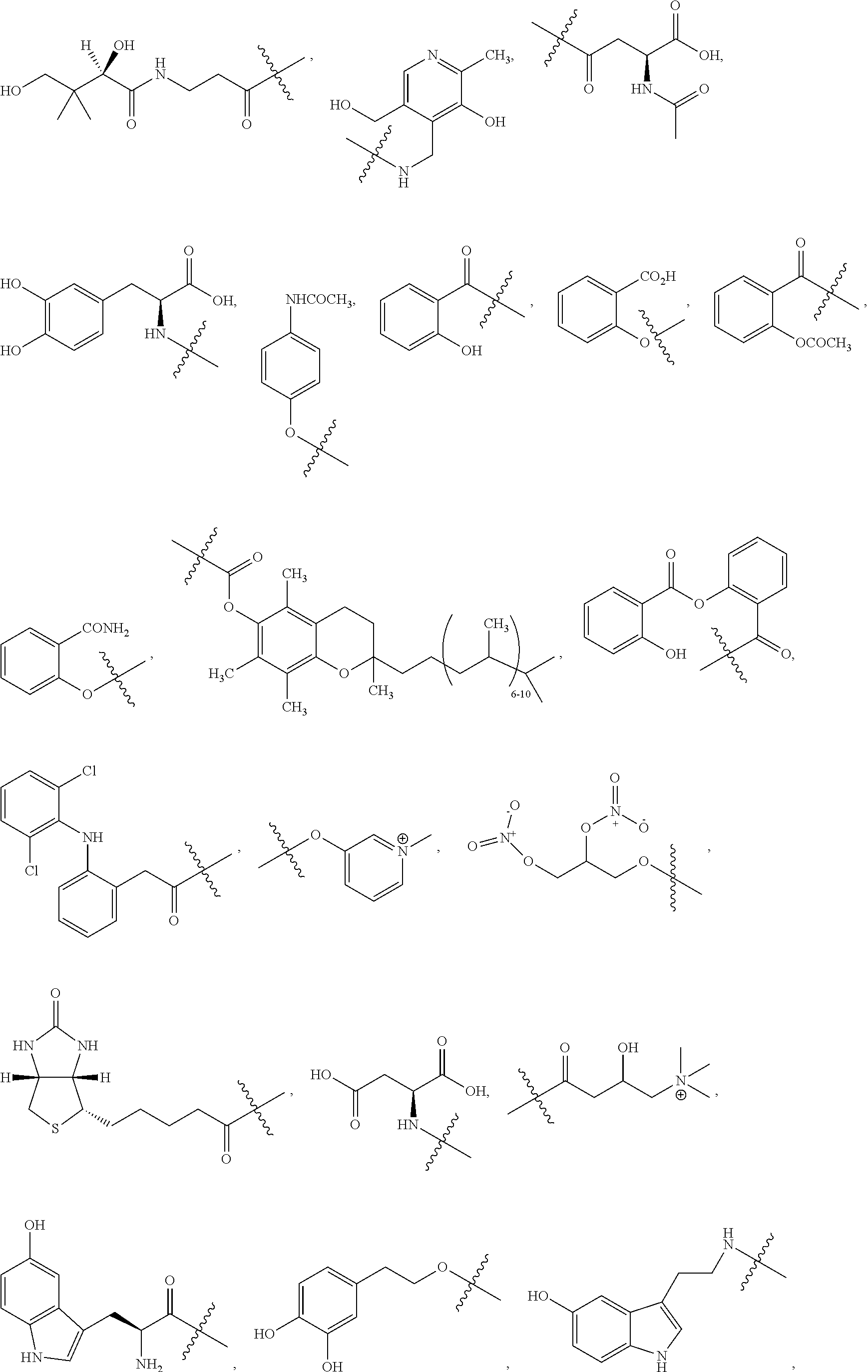 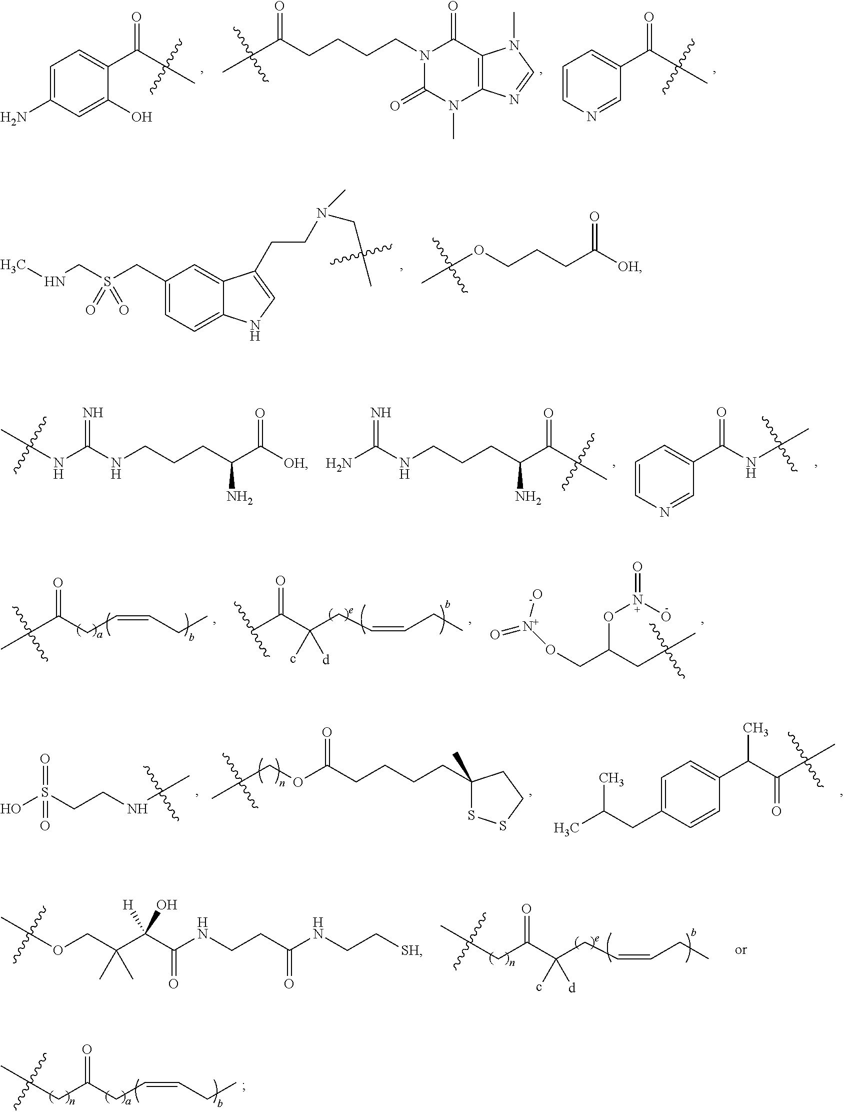
n is independently 1, 2, 3, 4 or 5;
a is independently 2, 3 or 7;
each b is independently 3, 5 or 6;
e is independently 1, 2 or 6;
c and d are each independently H, D, —OH, —OD, C₁-C₆-alkyl, —NH₂ or —COCH₃;
R⁴ represents H, D, CH₃CO, CD₃CO, CD₃, CH₃, 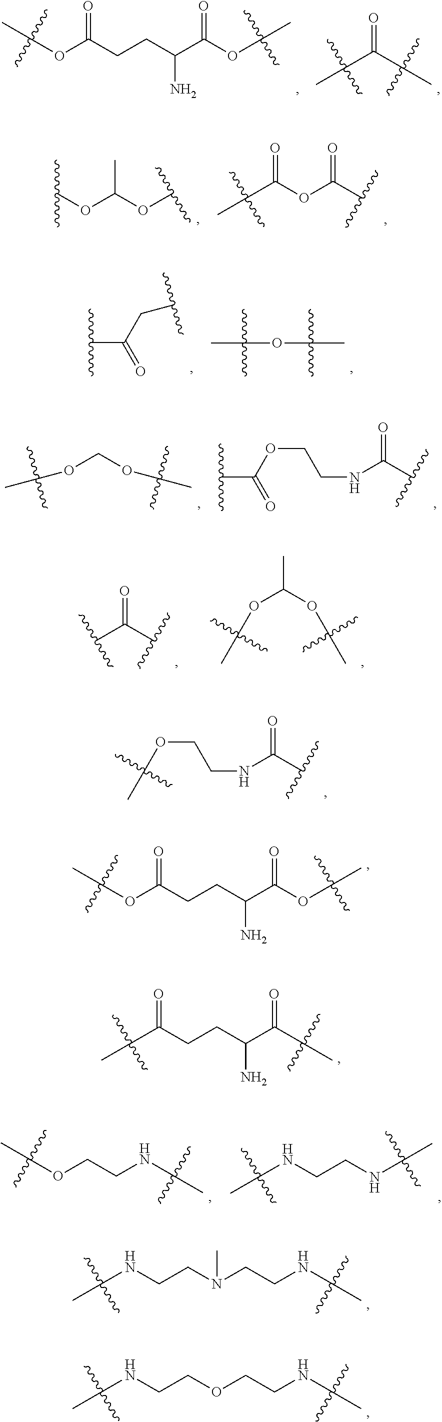 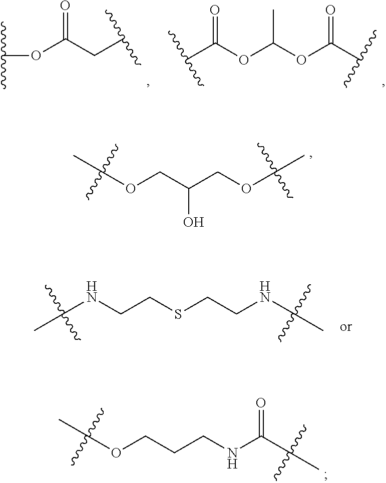
R⁵ represents D, CH₃CO, CD₃CO, CD₃, 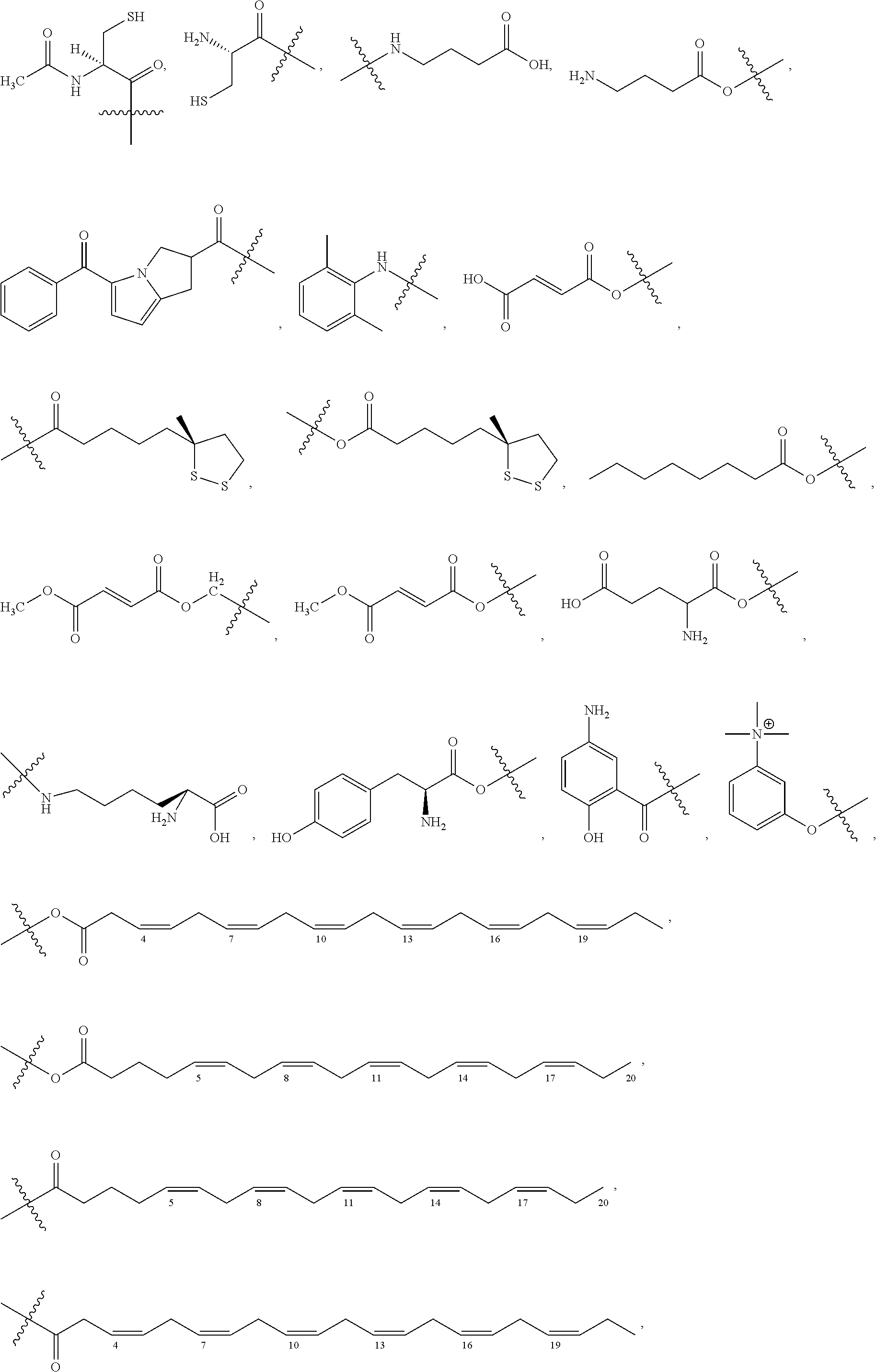 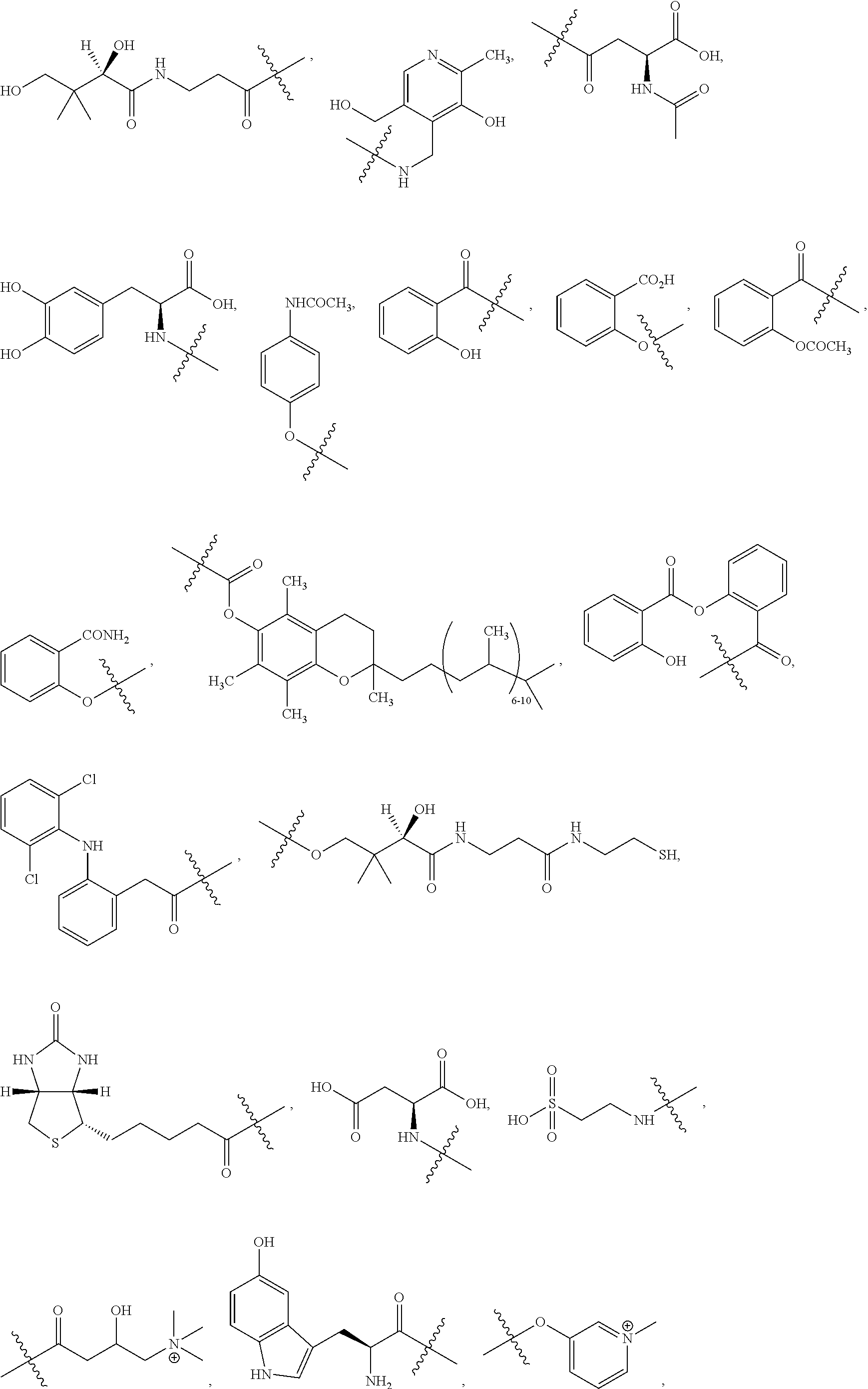 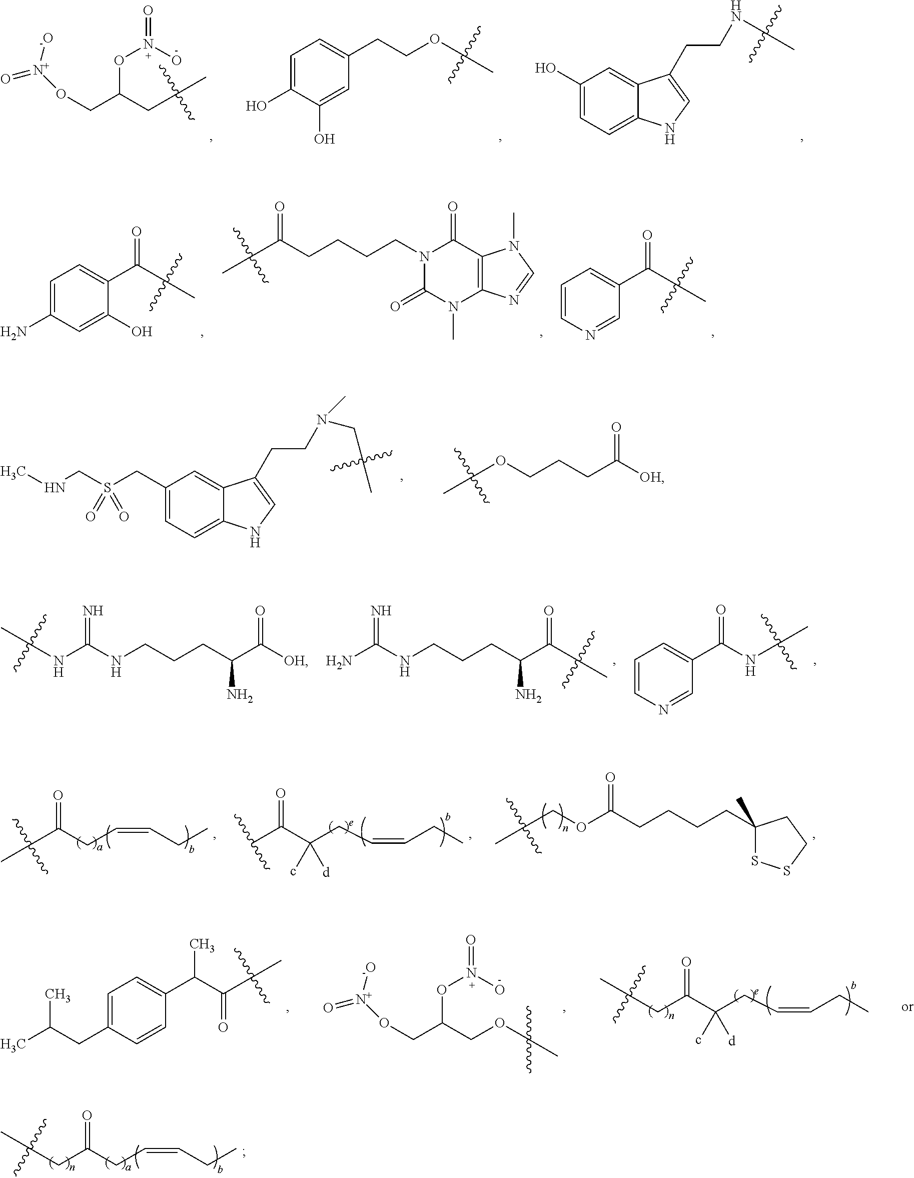
n is independently 1, 2, 3, 4 or 5;
a is independently 2, 3 or 7;
each b is independently 3, 5 or 6;
e is independently 1, 2 or 6;
c and d are each independently H, D, —OH, —OD, C₁-C₆-alkyl, —NH₂ or —COCH₃.

3. A compound of Formula III: 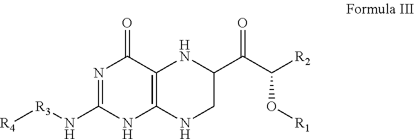
and pharmaceutically acceptable salts, hydrates, solvates, prodrugs, enantiomers, and stereoisomers thereof;
Wherein,
R¹, R², each independently represents H, D, CH₃CO, CD₃CO, CD₃, CH₃, 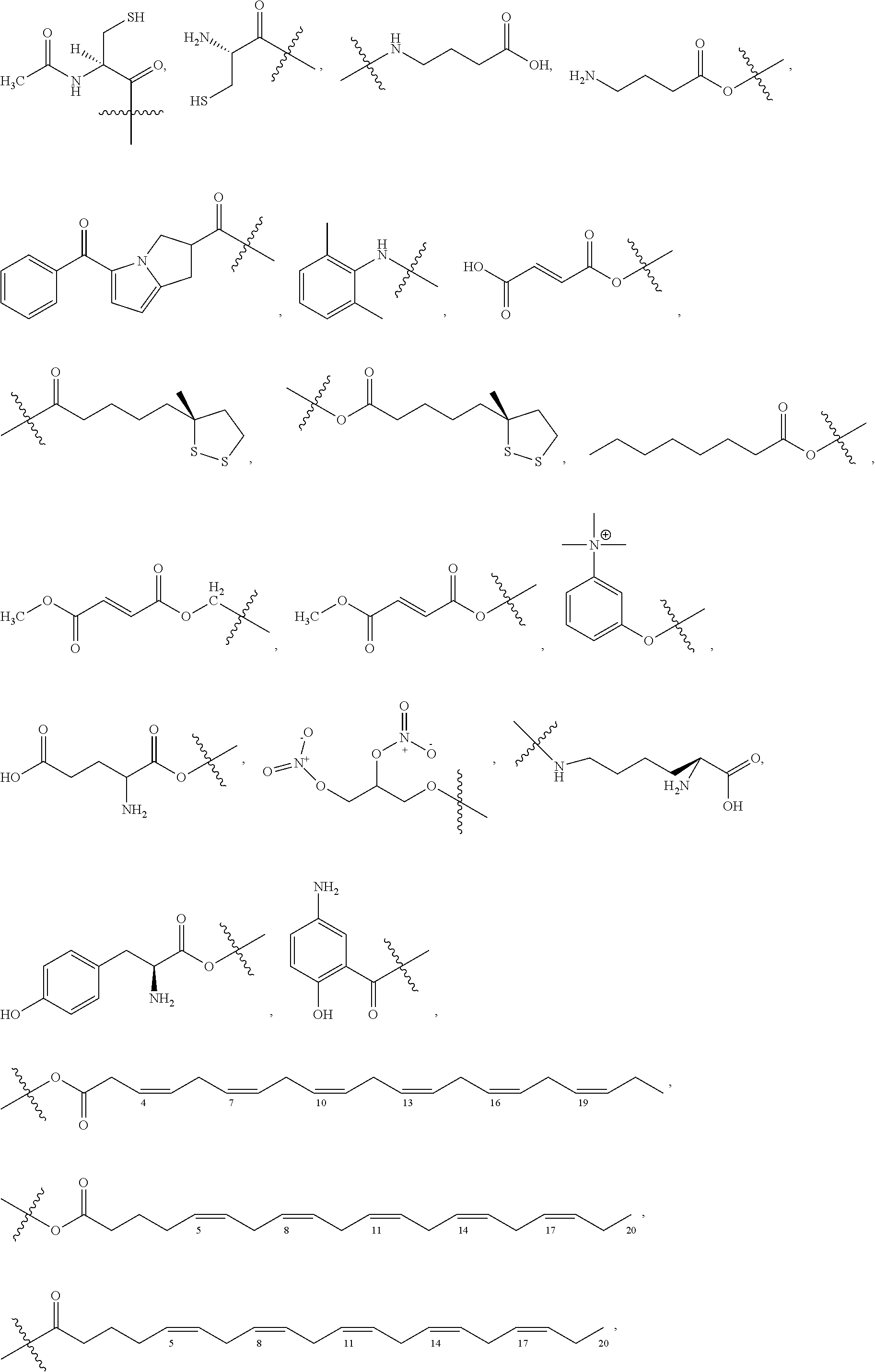 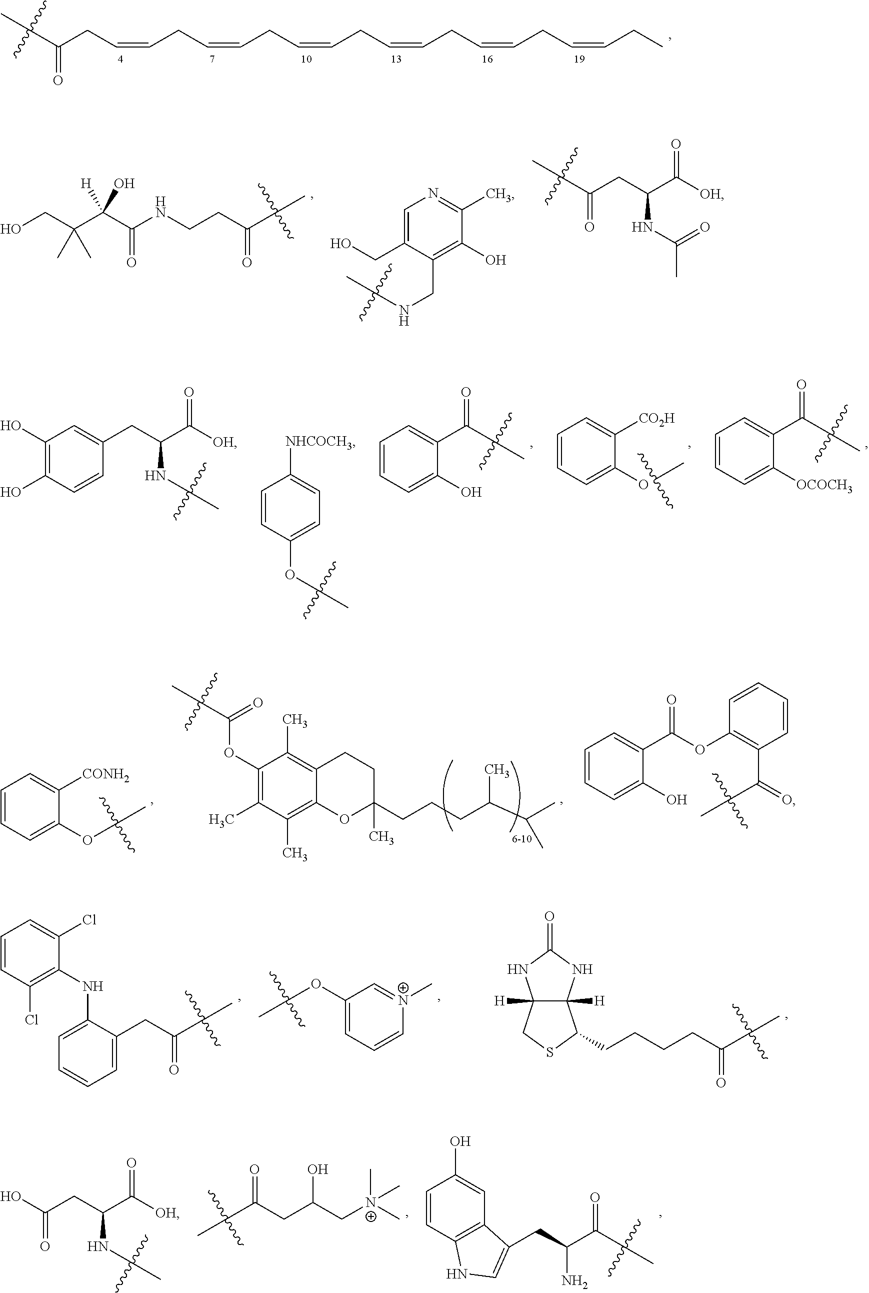 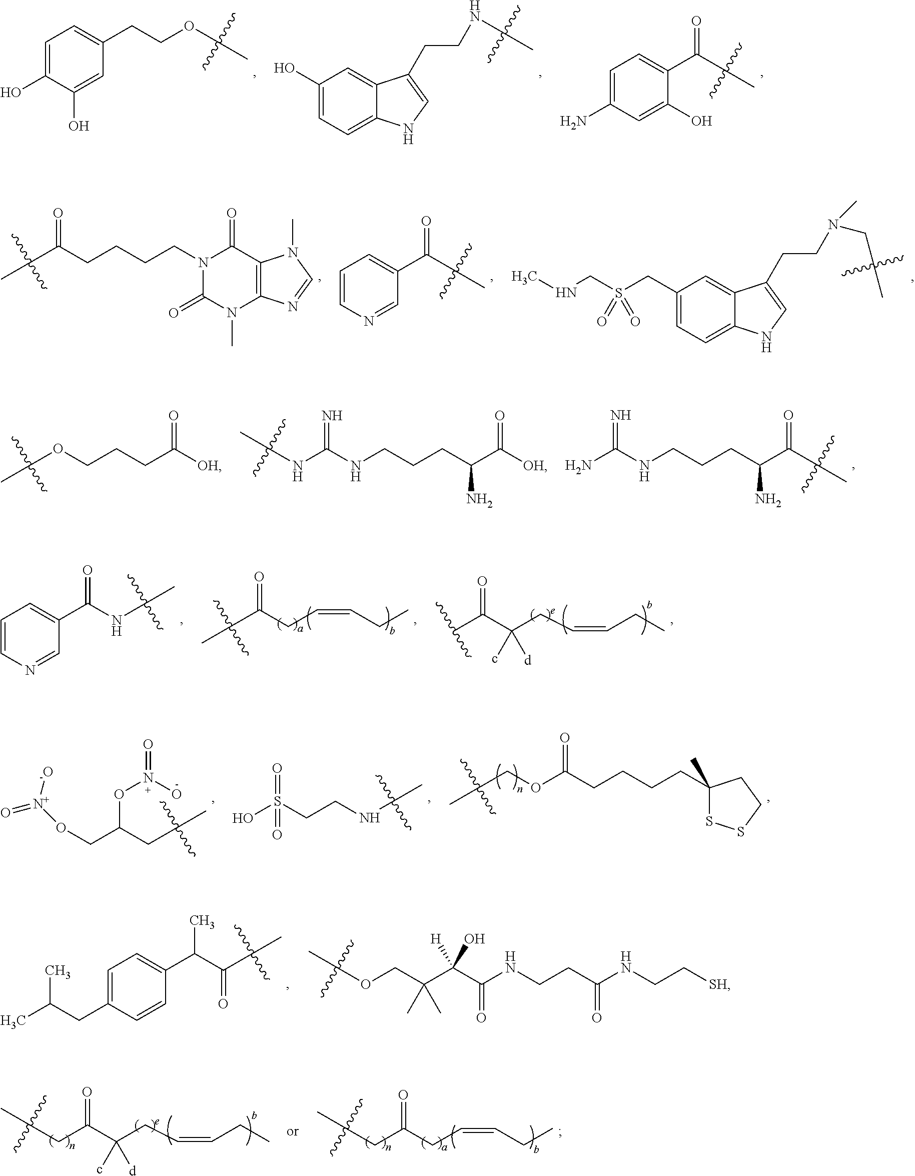
n is independently 1, 2, 3, 4 or 5;
a is independently 2, 3 or 7;
each b is independently 3, 5 or 6;
e is independently 1, 2 or 6;
c and d are each independently H, D, —OH, —OD, C₁-C₆-alkyl, —NH₂ or —COCH₃;
R³ represents H, D, CH₃CO, CD₃CO, CD₃, CH₃, 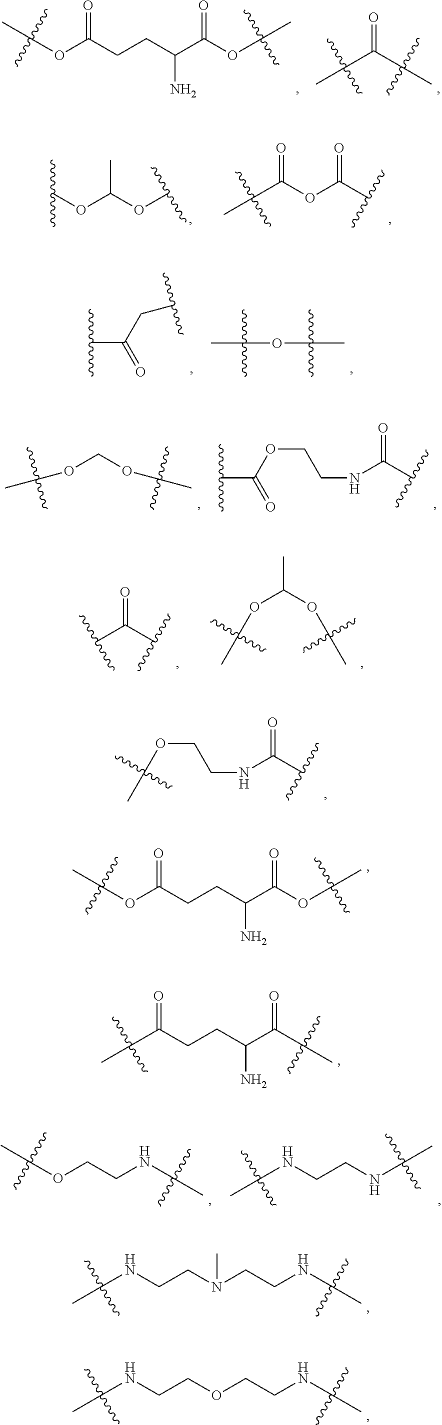 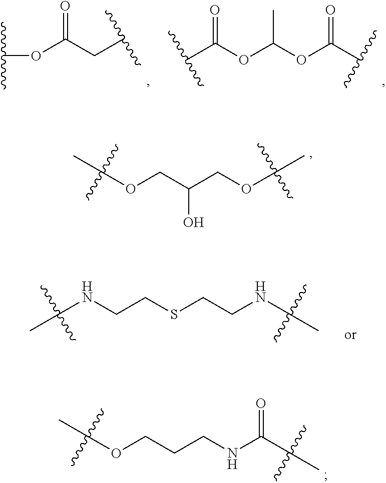
R⁴ represents D, CH₃CO, CD₃CO, CD₃, 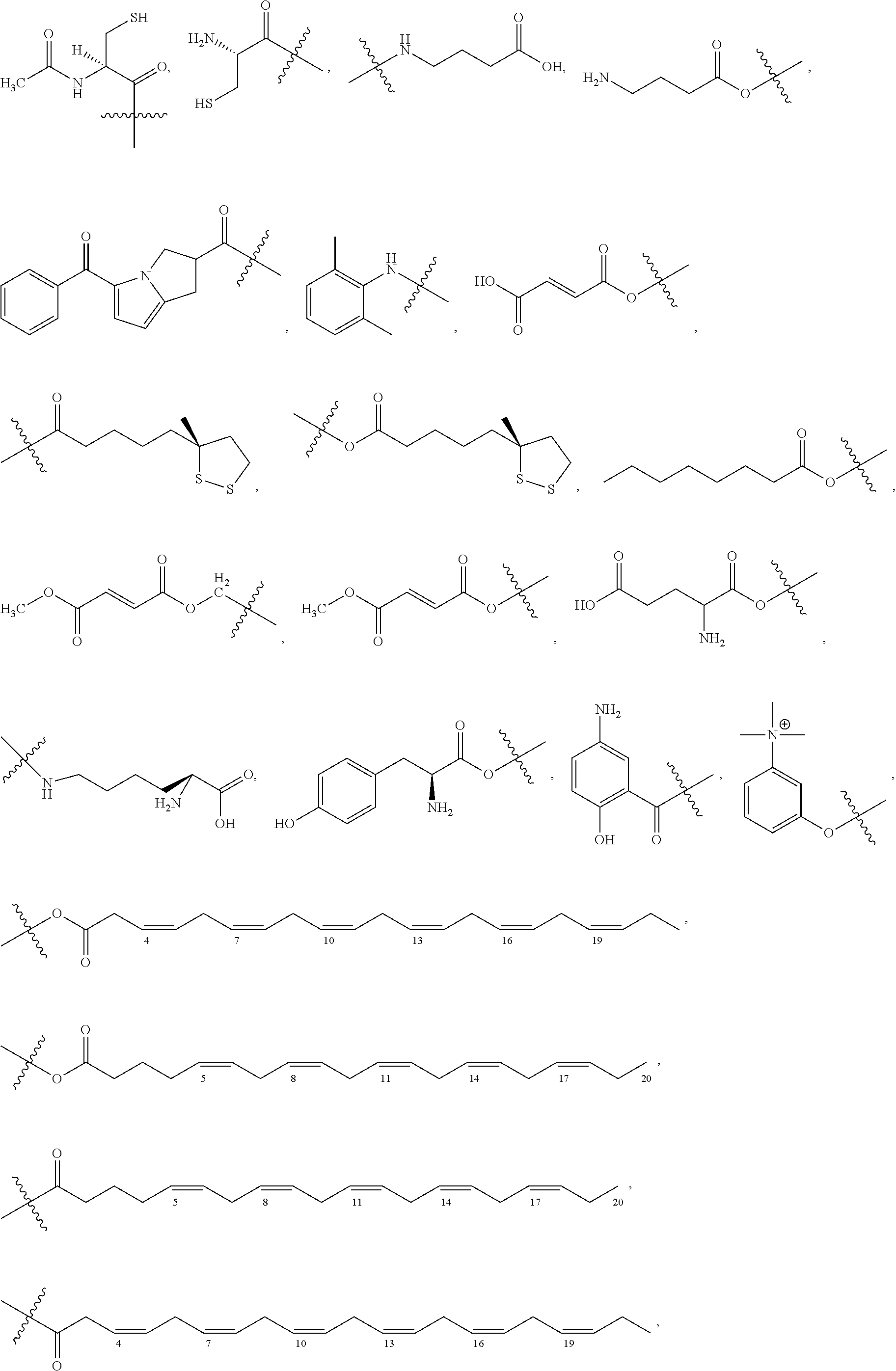 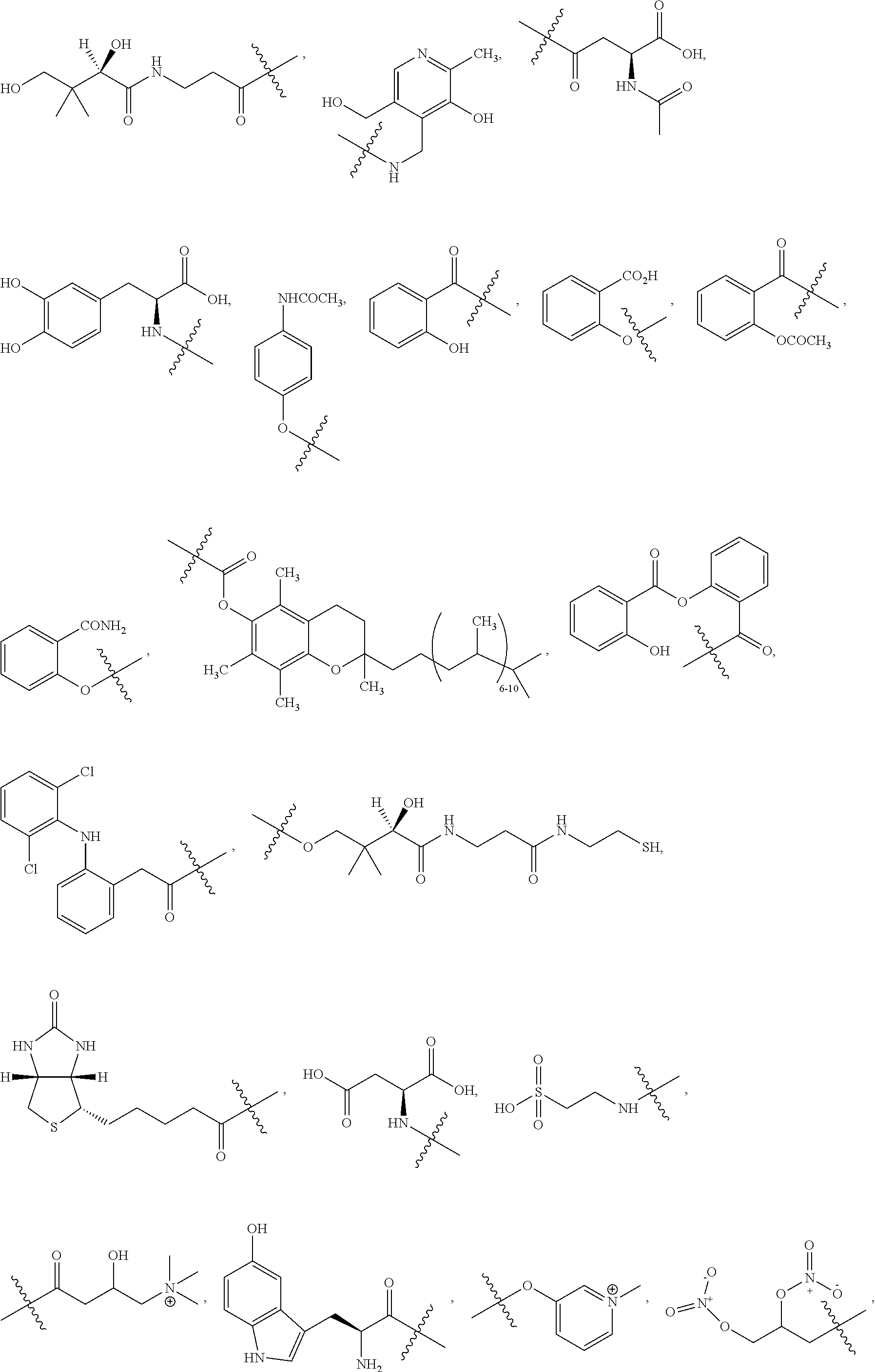 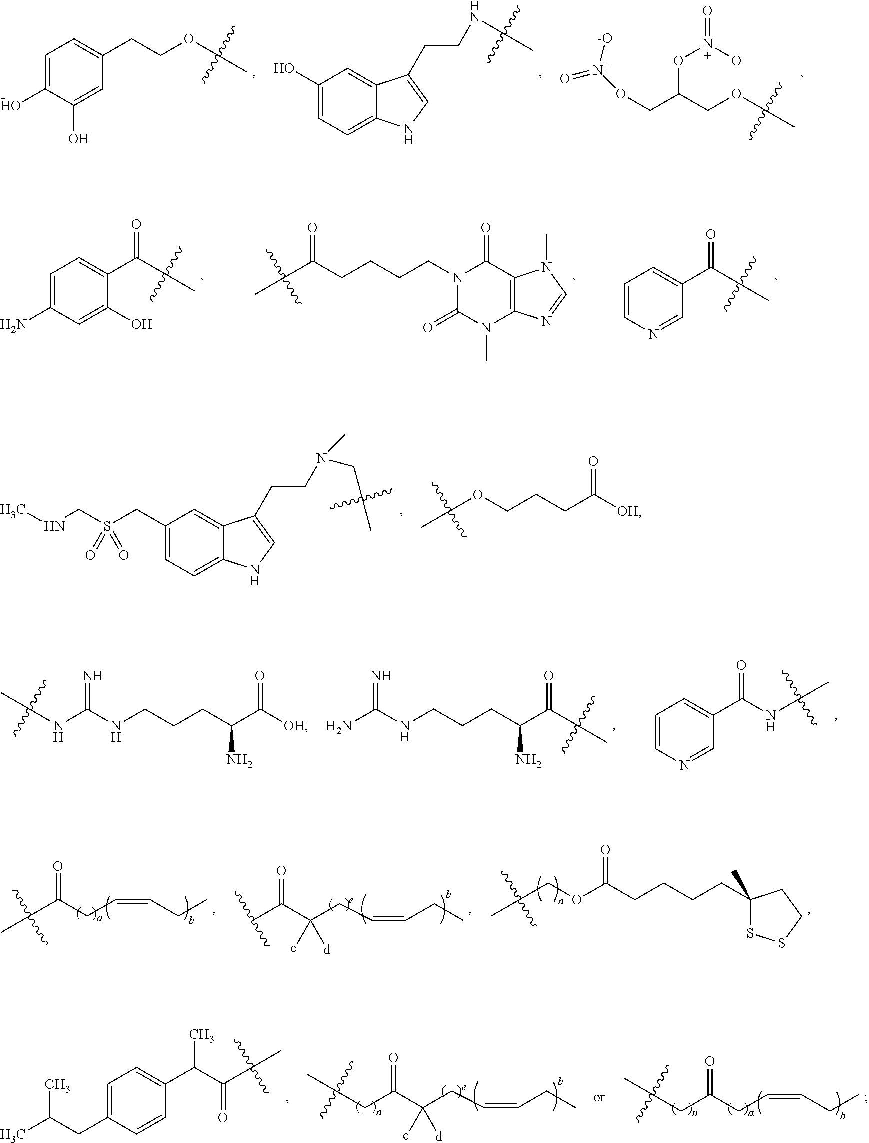
n is independently 1, 2, 3, 4 or 5;
a is independently 2, 3 or 7;
each b is independently 3, 5 or 6;
e is independently 1, 2 or 6;
c and d are each independently H, D, —OH, —OD, C₁-C₆-alkyl, —NH₂ or —COCH₃.

4. A Pharmaceutical composition comprising a compound of claim 1 and a pharmaceutically acceptable carrier.

5. A Pharmaceutical composition comprising a compound of claim 2 and a pharmaceutically acceptable carrier.

6. A Pharmaceutical composition comprising a compound of claim 3 and a pharmaceutically acceptable carrier.

7. The pharmaceutical composition of claim 4, which is formulated to treat the underlying etiology with an effective amount administering the patient in need by oral administration, delayed release or sustained release, transmucosal, syrup, topical, parenteral administration, injection, subdermal, oral solution, rectal administration, buccal administration or transdermal administration.

8. The pharmaceutical composition of claim 5, which is formulated to treat the underlying etiology with an effective amount administering the patient in need by oral administration, delayed release or sustained release, transmucosal, syrup, topical, parenteral administration, injection, subdermal, oral solution, rectal administration, buccal administration or transdermal administration.

9. The pharmaceutical composition of claim 6, which is formulated to treat the underlying etiology with an effective amount administering the patient in need by oral administration, delayed release or sustained release, transmucosal, syrup, topical, parenteral administration, injection, subdermal, oral solution, rectal administration, buccal administration or transdermal administration.

10. Compounds and compositions of claim 4 are formulated for the treatment of phenylketonuria, cardiovascular disease, autism, ADHD, hypertension, endothelial dysfunction and chronic kidney disease.

11. Compounds and compositions of claim 5 are formulated for the treatment of phenylketonuria, cardiovascular disease, autism, ADHD, hypertension, endothelial dysfunction and chronic kidney disease.

12. Compounds and compositions of claim 6 are formulated for the treatment of phenylketonuria, cardiovascular disease, autism, ADHD, hypertension, endothelial dysfunction and chronic kidney disease.
